SEQUENCE LISTING (92 sequences)

SEQ ID NO: 1 - protein, 21 aa, Homo sapiens
MALPVTALLLPLALLLHAARP

SEQ ID NO: 2 - protein, 15 aa, synthetic construct
  REGION 1..15: note = Synthiesized
GGGGSGGGGSGGGGS

SEQ ID NO: 3 - protein, 42 aa, Homo sapiens
KRGRKKLLYIFKQPFMRPVQTTQEEDGCSCRFPEEEEGGCEL

SEQ ID NO: 4 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 339 aa, Homo sapiens
AGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGGCGTGGCCGGGACCCTGAGATGGGGGGAAAGCCGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAA

SEQ ID NO: 6 - protein, 241 aa, synthetic construct
  REGION 1..241: note = Synthiesized
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 7 - protein, 241 aa, synthetic construct; identical to SEQ ID NO: 6
  REGION 1..241: note = Synthiesized

SEQ ID NO: 8 - protein, 119 aa, synthetic construct [sequence omitted]

SEQ ID NO: 9 - protein, 106 aa, synthetic construct
  REGION 1..106: note = Synthiesized
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIR

SEQ ID NO: 10 - protein, 107 aa, synthetic construct
  REGION 1..107: note = Synthiesized
DIQMTQSPSSLSASVGDRVTITCRASQDIRNYLNWYQQKPGKAPKLLIYYTSRLESGVPSRFSGSGSGTDYTLTISSLQPEDFATYYCQQGDYLPWTFGQGTKVEIK

SEQ ID NO: 11 - protein, 122 aa, synthetic construct [sequence omitted]

SEQ ID NO: 12 - protein, 111 aa, synthetic construct
  REGION 1..111: note = Synthiesized
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIK

SEQ ID NO: 13 - protein, 120 aa, synthetic construct
  REGION 1..120: note = Synthiesized
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSS

SEQ ID NO: 14 - protein, 108 aa, synthetic construct
  REGION 1..108: note = Synthiesized
DIQMTQSPSSLSASVGDRVTITCRASRPIWPFLEWYQQKPGKAPKLLIYFTSRLRHGVPSRFSGSGSGTCFTLTISSLQPEDFATYYCLQNVANPATFSQGTKVEIKR

SEQ ID NO: 15 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 16 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 17 - protein, 121 aa, synthetic construct [sequence omitted]

SEQ ID NO: 18 - protein, 109 aa, synthetic construct [sequence omitted]

SEQ ID NO: 19 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 20 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 21 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 22 - protein, 532 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 23 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 24 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 25 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 26 - protein, 503 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGSAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 27 - protein, 250 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 28 - protein, 243 aa, Homo sapiens
MCSRGWDSCLALELLLLPLSLLVTSIQGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSFGVEWIASWLVVTVPTILGLLLT

SEQ ID NO: 29 - protein, 454 aa, Homo sapiens
MTWRAAASTCAALLILLWALTTEGDLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFSIHWWPISFIGVGLVLLIVLIPWKKICNKSSSAYTPLKCILKHWNSFDTQTLKKEHLIFFCTRAWPSYQLQDGEAWPPEGSVNINTIQQLDVFCRQEGKWSEVPYVQAFFALRDNPDLCQCCRIDPALLTVTSGKSIDDNSTKSEKQTPREHSDAVPDAPILPVSPIWEPPPATTSTTPVLSSQPPTLLLPLQ

SEQ ID NO: 30 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 31 - protein, 383 aa, Homo sapiens
MGLGPVFLLLAGIFPFAPPGAAAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 32 - DNA, 574 bp, synthetic construct
  misc_feature 1..574: note = Synthiesized
  source 1..574: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 33 - protein, 19 aa, synthetic construct
  REGION 1..19: note = Synthiesized
ATNFSLLKQAGDVEENPGP

SEQ ID NO: 34 - protein, 18 aa, synthetic construct
  REGION 1..18: note = Synthiesized
EGRGSLLTCGDVEENPGP

SEQ ID NO: 35 - protein, 20 aa, synthetic construct
  REGION 1..20: note = Synthiesized
QCTNYALLKLAGDVESNPGP

SEQ ID NO: 36 - protein, 22 aa, synthetic construct
  REGION 1..22: note = Synthiesized
VKQTLNFDLLKLAGDVESNPGP

SEQ ID NO: 37 - protein, 38 aa, Homo sapiens
VSTISSFSPPGYQVSFCLVMVLLFAVDTGLYFSVKTNI

SEQ ID NO: 38 - protein, 55 aa, Homo sapiens
VDNLTLDGNPFLVPGTALPHEGSMNSGVVPACARSTLSVGVSGTLVLLQGARGFA

SEQ ID NO: 39 - protein, 39 aa, Homo sapiens
TTPNKGSGTTSGTTRLLSGHTCFTLTGLLGTLVTMGLLT

SEQ ID NO: 40 - protein, 37 aa, Homo sapiens
DLCNFNEQLENGGTSLSEKTVLLLVTPFLAAAWSLHP

SEQ ID NO: 41 - protein, 34 aa, Homo sapiens
MTVARPSVPAALPLLGELPRLLLLVLLCLPAVWG

SEQ ID NO: 42 - protein, 20 aa, Homo sapiens
MYRMQLLSCIALSLALVTNS

SEQ ID NO: 43 - protein, 22 aa, Homo sapiens
MDAMKRGLCCVLLLCGAVFVSP

SEQ ID NO: 44 - protein, 511 aa, synthetic construct
  REGION 1..511: note = Synthiesized
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 45 - protein, 617 aa, synthetic construct
  REGION 1..617: note = Synthiesized
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 46 - protein, 550 aa, synthetic construct
  REGION 1..550: note = Synthiesized
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGRCNKKGEQVGMSRPGLKPDLTGTSKSYVRSL

SEQ ID NO: 47 - protein, 599 aa, synthetic construct
  REGION 1..599: note = Synthiesized
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 48 - protein, 520 aa, synthetic construct
  REGION 1..520: note = Synthiesized
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGR

SEQ ID NO: 49 - protein, 665 aa, synthetic construct
  REGION 1..665: note = Synthiesized
MARSTLSKPLKNKVNPRGPLIPLILLMLRGVSTASPGSSPHQVYNITWEVTNGDRETVWATSGNHPLWTWWPDLTPDLCMLAHHGPSYWGLEYQSPFSSPPGPPCCSGGSSPGCSRDCEEPLTSLTPRCNTAWNRLKLDQTTHKSNEGFYVCPGPHRPRESKSCGGPDSFYCAYWGCETTGRAYWKPSSSWDFITVNNNLTSDQAVQVCKDNKWCNPLVIRFTDAGRRVTSWTTGHYWGLRLYVSGQDPGLTFGIRLRYQNLGPRVPIGPNPVLADQQPLSKPKPVKSPSVTKPPSGTPLSPTQLPPAGTENRLLNLVDGAYQALNLTSPDKTQECWLCLVAGPPYYEGVAVLGTYSNHTSAPANCSVASQHKLTLSEVTGQGLCIGAVPKTHQALCNTTQTSSRGSYYLVAPTGTMWACSTGLTPCISTTILNLTTDYCVLVELWPRVTYHSPSYVYGLFERSNRHKREPVSLTLALLLGGLTMGGIAAGIGTGTTALMATQQFQQLQAAVQDDLREVEKSISNLEKSLTSLSEVVLQNRRGLDLLFLKEGGLCAALKEECCFYADHTGLVRDSMAKLRERLNQRQKLFESTQGWFEGLFNRSPWFTTLISTIMGPLIVLLMILLFGPCILNRLVQFVKDRISVVQALVLTQQYHQLKPIEYEP

SEQ ID NO: 50 - protein, 905 aa, unidentified
MARSTLSKPPQDKINPWKPLIVMGVLLGVGDIQMTQSPSSLSASVGDRVTITCRASQDIRNYLNWYQQKPGKAPKLLIYYTSRLESGVPSRFSGSGSGTDYTLTISSLQPEDFATYYCQQGDYLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSAAAIEGRMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 51 - protein, 511 aa, synthetic construct; identical to SEQ ID NO: 44

SEQ ID NO: 52 - protein, 905 aa, synthetic construct; identical to SEQ ID NO: 50

SEQ ID NO: 53 - protein, 654 aa, synthetic construct
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 54 - protein, 905 aa, synthetic construct
MARSTLSKPPQDKINPWKPLIVMGVLLGVGMADIQMTQSPSSLSASVGDRVTITCRASQDIRNYLNWYQQKPGKAPKLLIYYTSRLESGVPSRFSGSGSGTDYTLTISSLQPEDFATYYCQQGDYLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSAAAIEGRESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 55 - protein, 1136 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGDYLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKGGGGSGGGGSGGGGSEVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSAAAIEGRMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 56 - protein, 905 aa, synthetic construct
MARSTLSKPPQDKINPWKPLIVMGVLLGVGDIQMTQSPSSLSASVGDRVTITCRASQDIRNYLNWYQQKPGKAPKLLIYYTSRLESGVPSRFSGSGSGTDYTLTISSLQPEDFATYYCQQGDYLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSAAAIEGRMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTIEGREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 57 - protein, 953 aa, synthetic construct
MARSTLSKPPQDKINPWKPLIVMGVLLGVGMADIQMTQSPSSLSASVGDRVTITCRASQDIRNYLNWYQQKPGKAPKLLIYYTSRLESGVPSRFSGSGSGTDYTLTISSLQPEDFATYYCQQGDYLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSMPRRRRSSSRPVRRRRRPRVSRRRRRRGGRRRRAAAIEGRESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 58 - protein, 244 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGDYLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 59 - protein, 246 aa, synthetic construct
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKGGGGSGGGGSGGGGSEVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSS

SEQ ID NO: 60 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 208 aa, synthetic construct [sequence omitted]

SEQ ID NO: 62 - protein, 595 aa, synthetic construct
GTCGAGCCGCGGCAATTCCGCCCCTCTCCCTCCCCCCCCCCTAACGTTACTGGCCGAAGCCGCTTGGAATAAGGCCGGTGTGCGTTTGTCTATATGTTATTTTCCACCATATTGCCGTCTTTTGGCAATGTGAGGGCCCGGAAACCTGGCCCTGTCTTCTTGACGAGCATTCCTAGGGGTCTTTCCCCTCTCGCCAAAGGAATGCAAGGTCTGTTGAATGTCGTGAAGGAAGCAGTTCCTCTGGAAGCTTCTTGAAGACAAACAACGTCTGTAGCGACCCTTTGCAGGCAGCGGAACCCCCCACCTGGCAACAGGTGCCTCTGCGGCCAAAAGCCACGTGTATAAGATACACCTGCAAAGGCGGCACAACCCCAGTGCCACGTTGTGAGTTGGATAGTTGTGGAAAGAGTCAAATGGCTCTCCTCAAGCGTATTCAACAAGGGGCTGAAGGATGCCCAGAAGGTACCCCATTGTATGGGATCTGATCTGGGGCCTCGGTGCACATGCTTTACATGTGTTTAGTCGAGGTTAAAAAAACGTCTAGGCCCCCCGAACCACGGGGACGTGGTTTTCCTTTGAAAAACACGATGATAAT

SEQ ID NO: 63 - protein, 152 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 64 - protein, 317 aa, synthetic construct
CGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTT

SEQ ID NO: 65 - protein, 560 aa, synthetic construct
MTVARPSVPAALPLLGELPRLLLLVLLCLPDIQMTQSPSSLSASVGDRVTITCRASQDIRNYLNWYQQKPGKAPKLLIYYTSRLESGVPSRFSGSGSGTDYTLTISSLQPEDFATYYCQQGDYLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGVDNLTLDGNPFLVPGTALPHEGSMNSGVVPACARSTLSVGVSGTLVLLQGARGFA

SEQ ID NO: 66 - protein, 55 aa, synthetic construct; identical to SEQ ID NO: 38

SEQ ID NO: 67 - protein, 29 aa, synthetic construct
SGTTRLLSGHTCFTLTGLLGTLVTMGLLT

SEQ ID NO: 68 - protein, 38 aa, synthetic construct; identical to SEQ ID NO: 37

SEQ ID NO: 69 - protein, 19 aa, synthetic construct
ATNFSLLKQAGDVEENPGP

SEQ ID NO: 70 - protein, 18 aa, synthetic construct
EGRGSLLTCGDVEENPGP

SEQ ID NO: 71 - protein, 20 aa, synthetic construct
QCTNYALLKLAGDVESNPGP

SEQ ID NO: 72 - protein, 560 aa, synthetic construct; identical to SEQ ID NO: 65

SEQ ID NO: 73 - protein, 595 aa, synthetic construct; identical to SEQ ID NO: 62

SEQ ID NO: 74 - protein, 562 aa, synthetic construct
MTVARPSVPAALPLLGELPRLLLLVLLCLPDIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKGGGGSGGGGSGGGGSEVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGVDNLTLDGNPFLVPGTALPHEGSMNSGVVPACARSTLSVGVSGTLVLLQGARGFA

SEQ ID NO: 75 - protein, 560 aa, synthetic construct; identical to SEQ ID NO: 65

SEQ ID NO: 76 - protein, 595 aa, synthetic construct; identical to SEQ ID NO: 62

SEQ ID NO: 77 - protein, 533 aa, synthetic construct
MATTMTVARPSVPAALPLLGELPRLLLLVLLCLPADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 78 - protein, 560 aa, synthetic construct; identical to SEQ ID NO: 65

SEQ ID NO: 79 - protein, 595 aa, synthetic construct; identical to SEQ ID NO: 62

SEQ ID NO: 80 - protein, 276 aa, synthetic construct
MTVARPSVPAALPLLGELPRLLLLVLLCLPVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNVSTISSFSPPGYQVSFCLVMVLLFAVDTGLYFSVKTNI

SEQ ID NO: 81 - protein, 1366 aa, synthetic construct
MTVARPSVPAALPLLGELPRLLLLVLLCLPDIQMTQSPSSLSASVGDRVTITCRASQDIRNYLNWYQQKPGKAPKLLIYYTSRLESGVPSRFSGSGSGTDYTLTISSLQPEDFATYYCQQGDYLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGVDNLTLDGNPFLVPGTALPHEGSMNSGVVPACARSTLSVGVSGTLVLLQGARGFAGSGQCTNYALLKLAGDVESNPGPMTVARPSVPAALPLLGELPRLLLLVLLCLPDIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKGGGGSGGGGSGGGGSEVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGVDNLTLDGNPFLVPGTALPHEGSMNSGVVPACARSTLSVGVSGTLVLLQGARGFAGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 82 - protein, 1920 aa, synthetic construct
MTVARPSVPAALPLLGELPRLLLLVLLCLPDIQMTQSPSSLSASVGDRVTITCRASQDIRNYLNWYQQKPGKAPKLLIYYTSRLESGVPSRFSGSGSGTDYTLTISSLQPEDFATYYCQQGDYLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGVDNLTLDGNPFLVPGTALPHEGSMNSGVVPACARSTLSVGVSGTLVLLQGARGFAGSGQCTNYALLKLAGDVESNPGPMTVARPSVPAALPLLGELPRLLLLVLLCLPDIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKGGGGSGGGGSGGGGSEVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGVDNLTLDGNPFLVPGTALPHEGSMNSGVVPACARSTLSVGVSGTLVLLQGARGFAGSGEGRGSLLTCGDVEENPGPMATTMTVARPSVPAALPLLGELPRLLLLVLLCLPADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLTGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 83 - protein, 781 aa, synthetic construct
MTVARPSVPAALPLLGELPRLLLLVLLCLPDIQMTQSPSSLSASVGDRVTITCRASQDIRNYLNWYQQKPGKAPKLLIYYTSRLESGVPSRFSGSGSGTDYTLTISSLQPEDFATYYCQQGDYLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGVDNLTLDGNPFLVPGTALPHEGSMNSGVVPACARSTLSVGVSGTLVLLQGARGFAGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 84 - protein, 633 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCREGSGATNFSLLKQAGDVEENPGPMALPVTALLLPLALLLHAARPDIQMTQSPSSLSASVGDRVTITCRASQDIRNYLNWYQQKPGKAPKLLIYYTSRLESGVPSRFSGSGSGTDYTLTISSLQPEDFATYYCQQGDYLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSRDPSTTTPAPRPPTPAPTIASQPLSLRPEACRPAAGGAVHTRGLDFACDIYIWAPLAGTCGVLLLSLVITLYC

SEQ ID NO: 85 - protein, 627 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCREGSGATNFSLLKQAGDVEENPGPMALPVTALLLPLALLLHAARPQVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSGGGSGGGSGGGSGGGSQIVLSQSPAILSASPGEKVTMTCRASSSVSYMHWYQQKPGSSPKPWIYATSNLASGVPARFSGSGSGTSYSLTISRVEAEDAATYYCQQWSSNPPTFGGGTKLETKRTTTPAPRPPTPAPTIASQPLSLRPEACRPAAGGAVHTRGLDFACDIYIWAPLAGTCGVLLLSLVITLYC

SEQ ID NO: 86 - protein, 632 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCREGSGATNFSLLKQAGDVEENPGPMALPVTALLLPLALLLHAARPDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSTTTPAPRPPTPAPTIASQPLSLRPEACRPAAGGAVHTRGLDFACDIYIWAPLAGTCGVLLLSLVITLYC

SEQ ID NO: 87 - protein, 511 aa, synthetic construct
MKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 88 - DNA, 3967 bp, synthetic construct
  source 1..3967: mol_type = other DNA
atgaccgtggcaagaccctcggtgcccgcggccttacccctgttgggcgagctgcctagactgctgctgctggtactcctgtgcctgcccgacattcagatgacacagagccctagcagcctgagcgctagcgtgggcgacagagtgaccatcacctgcagagctagccaagacatcagaaactacctgaactggtatcagcagaagcccggcaaggcccccaagctgctgatctactacacaagcagactggaaagcggcgtgccaagcagattcagcggcagcggcagcggcaccgactacaccctgaccatcagcagcctgcagcccgaggacttcgccacctactactgtcagcaaggcgactacctgccctggacttttggtcaaggtaccaaagttgagataaagggtggtggcggcagcggcgggggcggtagcggcggcggcgggtcggaggtgcaactggtagaaagcggtggcggcctagtgcagcccggcggcagcctgagactgagctgcgccgctagcggctacagcttcaccggctacaccatgaactgggtgagacaagcccccggcaagggcctggagtgggtggccctgatcaacccctacaagggcgtgagcacctacaatcagaagttcaaggacagattcaccatcagcgttgacaaaagcaaaaataccgcctacctgcagatgaacagcctgagagccgaggacaccgccgtgtactactgtgctagaagtggctattacggcgacagcgactggtacttcgacgtgtggggtcaagggaccctggtgaccgtgagcagcgagcccaaatcatgtgataagacccatacctgtccgccatgccccgctcccgagctactgggcggccctagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctgcccgcccccatcgagaagaccatcagcaaggccaaggggcagcctagagagccccaagtgtacaccctgccccctagcagagaggagatgaccaagaaccaagtgagcctgacctgcctggtgaagggcttctaccctagcgacatcgccgtggagtgggagagcaacgggcagcccgagaacaactacaagaccaccccccccgtgctggacagcgacggcagcttcttcctgtacagcaagctgaccgtggacaagagccgctggcagcaaggcaacgtgttcagctgcagcgtgatgcacgaggccctgcacaaccactacacacagaagagcctgagcctgagccccggcgtggacaacctgaccctcgacgggaacccctttctggtgcccggcacggccctgccccatgaaggctccatgaacagcggggtggtgcctgcctgtgcccgcagtactttgagcgtgggtgtgagtggaacactagtcctgctacaaggcgctagaggcttcgcctgagtcgagccgcggcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgaccgtggcaagaccctcggtgcccgcggccttacccctgttgggcgagctgcctagactgctgctgctggtactcctgtgcctgcccgacatcgaactgacccaaagccccgctagcctggccgtgagcctgggacaaagagctacaattagctgcagagccagcgagagcgtggaatattacgtgaccagcctgatgcagtggtaccagcagaagcccggccaacctcctaaactgctgatcttcgccgccagcaatgtggagagcggagtgcctgccagatttagcggaagcggaagcggaacaaacttcagcctgaacatccaccccgtggacgaggacgatgtggctatgtatttttgccagcagagcagaaaggtgccctacaccttcggcggcggaacaaaactggaaatcaagggaggcggcggcagcggaggaggaggaagcggaggaggaggaagcgaagttaagctgcaacaaagcggccccggactggtgacaccctctcaaagcctgagcattacatgtaccgtgagcggcttcagcctgagcgactatggagtgcattgggtgagacaaagccccggacagggactggagtggctgggagttatttgggctggaggaggaacaaactacaacagcgccctgatgagcagaaagagcatcagcaaggacaacagcaagagccaggtgttcctgaagatgaacagcctgcaggccgacgacaccgccgtgtattattgtgccagagacaagggctacagctactactacagcatggactactggggccagggcaccagcgtgacagtgtctagcgagcccaaatcatgtgataagacccatacctgtccgccatgccccgctcccgagctactgggcggccctagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctgcccgcccccatcgagaagaccatcagcaaggccaaggggcagcctagagagccccaagtgtacaccctgccccctagcagagaggagatgaccaagaaccaagtgagcctgacctgcctggtgaagggcttctaccctagcgacatcgccgtggagtgggagagcaacgggcagcccgagaacaactacaagaccaccccccccgtgctggacagcgacggcagcttcttcctgtacagcaagctgaccgtggacaagagccgctggcagcaaggcaacgtgttcagctgcagcgtgatgcacgaggccctgcacaaccactacacacagaagagcctgagcctgagccccggcgtggacaacctgaccctcgacgggaacccctttctggtgcccggcacggccctgccccatgaaggctccatgaacagcggggtggtgcctgcctgtgcccgcagtactttgagcgtgggtgtgagtggaacactagtcctgctacaaggcgctagaggcttcgcctga

SEQ ID NO: 89 - protein, 700 aa, synthetic construct
MALPVTALLLPLALLLHAARPDIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKGGGGSGGGGSGGGGSEVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSRDPSTTTPAPRPPTPAPTIASQPLSLRPEACRPAAGGAVHTRGLDFACDIYIWAPLAGTCGVLLLSLVITLYCGSGATNFSLLKQAGDVEENPGPMALPVTALLLPLALLLHAARPDIQMTQSPSSLSASVGDRVTITCRASQDIRNYLNWYQQKPGKAPKLLIYYTSRLESGVPSRFSGSGSGTDYTLTISSLQPEDFATYYCQQGDYLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSRDPSTTTPAPRPPTPAPTIASQPLSLRPEACRPAAGGAVHTRGLDFACDIYIWAPLAGTCGVLLLSLVITLYC

SEQ ID NO: 90 - DNA, 2103 bp, synthetic construct
  source 1..2103: mol_type = other DNA
atggccttaccagtgaccgccttgctcctgccgctggccttgctgctccacgccgccaggccggacatcgaactgacccaaagccccgctagcctggccgtgagcctgggacaaagagctacaattagctgcagagccagcgagagcgtggaatattacgtgaccagcctgatgcagtggtaccagcagaagcccggccaacctcctaaactgctgatcttcgccgccagcaatgtggagagcggagtgcctgccagatttagcggaagcggaagcggaacaaacttcagcctgaacatccaccccgtggacgaggacgatgtggctatgtatttttgccagcagagcagaaaggtgccctacaccttcggcggcggaacaaaactggaaatcaagggaggcggcggcagcggaggaggaggaagcggaggaggaggaagcgaagttaagctgcaacaaagcggccccggactggtgacaccctctcaaagcctgagcattacatgtaccgtgagcggcttcagcctgagcgactatggagtgcattgggtgagacaaagccccggacagggactggagtggctgggagttatttgggctggaggaggaacaaactacaacagcgccctgatgagcagaaagagcatcagcaaggacaacagcaagagccaggtgttcctgaagatgaacagcctgcaggccgacgacaccgccgtgtattattgtgccagagacaagggctacagctactactacagcatggactactggggccagggcaccagcgtgacagtgtctagcagagatccatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcggttccggagctaccaacttttccctcctcaagcaagccggcgatgtcgaggagaatcccggacccatggccttaccagtgaccgccttgctcctgccgctggccttgctgctccacgccgccaggccggacattcagatgacacagagccctagcagcctgagcgctagcgtgggcgacagagtgaccatcacctgcagagctagccaagacatcagaaactacctgaactggtatcagcagaagcccggcaaggcccccaagctgctgatctactacacaagcagactggaaagcggcgtgccaagcagattcagcggcagcggcagcggcaccgactacaccctgaccatcagcagcctgcagcccgaggacttcgccacctactactgtcagcaaggcgactacctgccctggacttttggtcaaggtaccaaagttgagataaagggtggtggcggcagcggcgggggcggtagcggcggcggcgggtcggaggtgcaactggtagaaagcggtggcggcctagtgcagcccggcggcagcctgagactgagctgcgccgctagcggctacagcttcaccggctacaccatgaactgggtgagacaagcccccggcaagggcctggagtgggtggccctgatcaacccctacaagggcgtgagcacctacaatcagaagttcaaggacagattcaccatcagcgttgacaaaagcaaaaataccgcctacctgcagatgaacagcctgagagccgaggacaccgccgtgtactactgtgctagaagtggctattacggcgacagcgactggtacttcgacgtgtggggtcaagggaccctggtgaccgtgagcagcagagatccatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctga

SEQ ID NO: 91 - protein, 21 aa, synthetic construct
MALPVTALLLPLALLLHAARP

SEQ ID NO: 92 - protein, 242 aa, synthetic construct
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSGGGSGGGSGGGSGGGSQIVLSQSPAILSASPGEKVTMTCRASSSVSYMHWYQQKPGSSPKPWIYATSNLASGVPARFSGSGSGTSYSLTISRVEAEDAATYYCQQWSSNPPTFGGGTKLETKR

(19) United States
(12) Patent Application Publication
(10) Pub. No.: US 2026/0060984 A1
(43) Pub. Date: Mar. 5, 2026

(54) Recombinant Viral Particle for Gene and/or Cellular Therapy

(71) Applicants: Innovative Cellular Therapeutics Holdings, Ltd., George Town (KY); Innovative Cellular Therapeutics, Inc., Rockville, MD (US)

(72) Inventors: Zhiyuan Cao, Rockville, MD (US); Wei Ding, Shanghai (CN); Guiting Han, Shanghai (CN); Chengfei Pu, Shanghai (CN); Dongqi Chen, Shanghai (CN); Le Tian, Rockville, MD (US); Lei Xiao, Rockville, MD (US)

(73) Assignees: Innovative Cellular Therapeutics Holdings, Ltd., George Town (KY); Innovative Cellular Therapeutics, Inc., Rockville, MD (US)

(21) Appl. No.: 18/994,494

(22) PCT Filed: Jul. 14, 2023
(86) PCT No.: PCT/US2023/027819

§ 371 Date: Jan. 14, 2025

Related U.S. Application Data
(63) Continuation of application No. 17/812,521, filed on Jul. 14, 2022, now abandoned.
(60) Provisional application No. 63/373,281, filed on Aug. 23, 2022.
(60) Provisional application No. 63/381,464, filed on Oct. 28, 2022.
(60) Provisional application No. 63/483,167, filed on Feb. 3, 2023.
(60) Provisional application No. 63/496,594, filed on Apr. 17, 2023.

(51) Int. Cl. A61K 31/519; A61K 38/17; A61K 38/19; A61K 38/20; A61K 38/21

(52) U.S. Cl. CPC A61K 31/519; A61K 38/1774; A61K 38/195; A61K 38/20; A61K 38/204; A61K 38/2086; A61K 38/217

(57) ABSTRACT

The present disclosure relates to systems and methods for immune therapy. For example, a method can be used to enhance the proliferation of chimeric antigen receptor (CAR) T cells in a subject, The method comprises administering to the subject an effective amount of a pharmaceutical composition comprising one or more lymphocyte activation agents and one or more recombinant viral particles comprising a polynucleotide encoding a CAR, wherein the proliferation of CAR T cells in the subject is greater than in a subject administered with the one or more recombinant viral particles but without the lymphocyte activation agent.

### CROSS REFERENCE TO RELATED APPLICATIONS

This application claims the benefit of U.S. application Ser. No. 17/812,521, filed Jul. 14, 2022; U.S. Provisional Application No. 63/373,281, filed Aug. 23, 2022; U.S. Provisional Application No. 63/381,464, filed Oct. 28, 2022; U.S. Provisional Application No. 63/483,167, filed Feb. 3, 2023; and U.S. Provisional Application No. 63/496,594, filed Apr. 17, 2023, all of which are incorporated herein by reference in their entirety.

### SEQUENCE LISTING INFORMATION

A computer readable XML document, entitled “Sequence Listing.XML,” created on or about Jul. 13, 2023, with a file size of about 126,892 bytes, contains the sequence listing for this application and is hereby incorporated by reference in its entirety.

### TECHNICAL FIELD

The present disclosure relates to compositions and methods for expanding and maintaining modified cells including genetically modified cells and uses thereof in the treatment of diseases, including cancer.

### BACKGROUND

Chimeric antigen receptor (CAR) T cells, such as CAR T lymphocytes identify tumor-specific markers and play a direct role in killing tumor cells. Since the first generation of CAR molecules was constructed, T cells expressing various CAR molecules have been widely used for treating diseases, such as cancers. However, one of the challenges of CAR T cell therapy is the development of efficient technologies and cost-effective manufacturing platforms to allow safe and effective clinical treatments.

### SUMMARY

Embodiments relate to a system for immune therapy, the system comprising: a sample processing module configured to obtain blood cells such as CD3+ cells from a blood sample from a subject; a cell incubation module configured to activate the blood cell and introduce a vector into the blood cells, and a cell infusion module configured to infuse at least a portion of the blood into the subject. In embodiments, the system is a closed and/or automated system for immune therapy including gene and/or cellular therapy.

This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIG. 1 shows exemplary constructs including viral vectors and packaging multiplicity of infection (MOI). Exemplary target vectors include vectors encoding chimeric antigen receptor (CAR), such as GCC CAR and CD19CAR. Exemplary lentivirus packaging vectors include pLP1 and pLP2.

FIG. 2 shows exemplary constructs including viral vectors.

FIG. 3 shows exemplary constructs including viral vectors.

FIG. 4 shows exemplary constructs including viral vectors.

FIGS. 5A and 5B compares conventional CAR T therapy (A) and novel CAR T therapy (B).

FIGS. 6A and 6B show a schematic view of gene therapy and packaging of cells for producing viral particles for gene therapy.

FIGS. 7A and 7B show a schematic view of gene therapy and packaging of cells for producing viral particles for gene therapy.

FIGS. 8, 9, 10, 11, 12, 13, and 14 show schematic views of embodiments of cellular therapeutic instruments/systems. Examples of CAR expression vectors 2516 can include at least one of the vectors listed in Table 3 or the vector of Embodiment 5402 (FIG. 16). In embodiments, the expression vector 2516 can include a vector that reprograms T cells to obtain CAR T cells listed in Table 4.

FIG. 15 shows gene delivery and expression using various lentivirus vectors coated with different types of envelopes.

FIG. 16 shows schematic views of expression vector 5402 and CAR T cell, for example, expressing GCC, CD19, and IL12, shown in Table 4.

FIGS. 17, 18, 19, 20, 21, 22, 23, and 24 show flow cytometry results of GCC CAR (single lentiviral vector) expression as well as CD19 CAR and GCC CAR (two separate vectors encoding two different CARs) expression in T cells using conventional and novel CAR T methods described therein. The novel CAR T methods involve using packaging cell-activating element 1340.

FIG. 25 shows histograms of CAR (GCC CAR, CD19CAR, and CoupledCAR®) expression in CAR T cells generated using conventional techniques and novel methods. NC is negative control. NT is non-treated.

FIG. 26 shows histograms comparing the expression of different types of CAR T cells generated using conventional techniques and novel methods.

FIG. 27 shows a histogram comparing the expression of GCC CAR in gated T cells generated using various methods.

FIG. 28 shows histograms comparing the expression of different types of T cells generated using various methods.

FIG. 29 shows histograms comparing cytokine release by CAR T cells generated using various methods.

FIG. 30 shows a histogram comparing the killing ability of CAR T cells generated using various methods.

FIG. 31 shows flow cytometry results comparing the proliferation capacity of CAR T cells generated using various methods.

FIG. 32 shows a summary of CAR T therapy in Patient 01.

FIGS. 33A, 33B, and 33C show the enrichment of a specific type of T cell by itself after CAR T cells infusion on Patient 01.

FIG. 34 shows a summary of CAR T therapy in Patient 02.

FIGS. 35A, 35B, and 35C show the enrichment of a specific type of T cell by itself after CAR T cells infusion on Patient 02.

FIG. 36 shows PET-CT scans of Patient 02 before and one month after the CAR T treatment.

FIG. 37 shows a summary of CAR T therapy on Patient 03 (non-responder).

FIGS. 38A, 38B, and 38C show of the non-enrichment of any specific type of T cell after CAR T cells infusion on Patient 03.

FIGS. 39, 40, and 41 show novel design of exemplary vectors and their use.

FIG. 42 shows that both the conventional CAR-T (developed through Prodigy technology) and 1340 CAR and 1346 CAR can significantly enhance the in vivo expansion of CD19 CAR-T cells through B cells. Notably, 1346 outperforms 1340. The experiment was performed using Cellular Therapeutic Instrument 2400 and mice. X axis: days after infusing into mice; Y axis: cell number/ml blood; and **** indicates statistical significance is <0.001; and ns is not significant. NT is non-transduced and MOCK is injection without cells.

FIG. 43 shows that both the conventional CAR-T (via Prodigy technology) and 1340 CAR and 1346 CAR can remarkably enhance the in vivo expansion of GCC CAR-T cells through B cells, with 1346 performing better than 1340. The experiment was performed using Cellular Therapeutic Instrument 2400 and mice.

FIG. 44 shows that both the conventional CAR-T (Prodigy technology) and 1340 CAR and 1346 CAR can significantly amplify the dual-positive cells of GCC CAR and CD19 CAR in vivo via B cells. Once again, 1346 exhibits superior performance over 1340. The experiment was performed using Cellular Therapeutic Instrument 2400 and mice.

FIG. 45 shows CAR expression using various lentiviral particles.

FIG. 46 shows cellular expression activation after co-cultivation.

FIG. 47 shows cytokine release after co-cultivation.

FIG. 48 shows exemplary constructs.

FIG. 49 shows schematic diagrams of the expression of proteins encoded by 1340 and 1641/1645/1646 on the surface of the virus.

### DETAILED DESCRIPTION

When a lentivirus (such as HIV-1) transfects host cells, it can randomly and stably integrate the carried foreign genes into the host cell genome to achieve stable and long-term expression of the target gene, which is very suitable for immunotherapy. Lentivirus can transfect dividing and non-dividing mammalian cells, but the transfection efficiency of resting cells (such as resting T cells) is very low. The transfection of T cells usually requires separating the T cells first, activating the T cells through TCR or factor signals, and then transfection. This process prolongs the time of transfection.

In embodiments, the surface of lentiviral vectors is genetically modified so that the surface of the virus particle carries a polypeptide such as a T cell-activating element or NK cell-activating element so that the virus can activate the resting cells in the peripheral blood, thereby improving transfection efficiency of immune cells. In addition, after the virus is injected into the body of a subject through a vein, the virus packaged by vesicular stomatitis virus G envelope protein (VSVG) will be affected by the complement proteins in the blood of the subject and become inactivated. To make the virus packaged by VSVG more stable in the body of the subject, the virus particles can be modified with polyethylene glycol (PEG). The VSVG mutants, (K66T+S162T+T230N+T368A) can also be used.

In the whole blood transfection process, the immune cells can be activated while transfecting the immune cells without the addition of cell-stimulating reagents, and the efficiency of virus infection can be improved. For example, a virus packaging system uses transfer plasmid, Gag-Pol, Rev, and VSVG to infect 293T cells to obtain virus particles. In order to carry the cell-activating element on the surface of the virus particle, VSVG needs to be modified. The modification method is to connect the cell-activating element to the N terminus of the VSVG. The envelope glycoprotein of other viruses, such as Moloney measles virus (Mo-MV) envelope glycoprotein, Nipah virus (NiV), or amphotropic murine leukemia virus (MLV-A) glycoprotein, and others, can be used to replace the VSVG. The modification involves connecting the cell-activating element to the N-terminus or C-terminus of the glycoprotein. Among them, the Mo-MV envelope glycoprotein is composed of hemagglutinin (H) and fusion (F) proteins, and the cell-activating element is connected to the N-terminus of the H protein, so the virus packaging system consists of 5 plasmids (main plasmid, Gag-Pol, Rev, H, F).

In embodiments, 293T cells are initially transfected with a plasmid that encoding a cell activating element serving as a transfer vector, as well as additional plasmids carrying genes for Gag-Pol, Rev, and VSVG. This results in the production of a virus that expresses the cell activating element on its surface. These newly produced viruses, carrying the cell activating element, are then used to infect a fresh batch of 293T cells. This process ensures that these cells become “modified” and now stably express the cell-activating element on their surface. The expression of the cell-activating element in these 293T cells is confirmed through a screening process. The successfully modified 293T cells are used for the next stage. Finally, these modified 293T cells, which stably express the cell-activating element, are used to package a lentivirus. This is done by transfecting these cells with the necessary plasmids (including a main plasmid, Gag-Pol, Rev, and an envelope (Env) protein). The resulting lentivirus produced by these cells will have the cell-activating element displayed on its surface.

Unless defined otherwise, all technical and scientific terms used herein have the same meaning as commonly understood by those of ordinary skill in the art to which the disclosure belongs. Although any method and material similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and refers to monoclonal antibodies (including full length monoclonal antibodies), polyclonal antibodies, multi-specific antibodies (e.g., bispecific antibodies), and antibody fragments so long as they exhibit the desired biological activity or function. The antibodies in the present disclosure may exist in a variety of forms including, for example, polyclonal antibodies, monoclonal antibodies, and Fv, Fab, Fab′ and F(ab′)₂ and fragments, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragments” refers to a portion of a full length antibody, for example, the antigen binding or variable region of the antibody. Other examples of antibody fragments include Fab, Fab′, F(ab′)₂, and Fv fragments; diabodies; linear antibodies; single-chain antibody molecules; and multi-specific antibodies formed from antibody fragments.

The term “Fv” refers to the minimum antibody fragment which contains a complete antigen-recognition and -binding site. This fragment consists of a dimer of one heavy- and one light-chain variable region domain in tight, non-covalent association. From the folding of these two domains emanates six hypervariable loops (3 loops each from the H and L chain) that contribute the amino acid residues for antigen binding and confer antigen binding specificity to the antibody. However, even a single variable domain (or half of an Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and A light chains refer to the two major antibody light chain isotypes.

The term “synthetic antibody” refers to an antibody which is generated using recombinant DNA technology, such as, for example, an antibody expressed by a bacteriophage. The term also includes an antibody which has been generated by the synthesis of a DNA molecule encoding the antibody and the expression of the DNA molecule to obtain the antibody, or to obtain an amino acid encoding the antibody. The synthetic DNA is obtained using technology that is available and well known in the art.

The term “antigen” refers to a molecule that provokes an immune response, which may involve either antibody production, or the activation of specific immunologically-competent cells, or both. Antigens include any macromolecule, including all proteins or peptides, or molecules derived from recombinant or genomic DNA. For example, DNA including a nucleotide sequence or a partial nucleotide sequence encoding a protein or peptide that elicits an immune response, and therefore, encodes an “antigen” as the term is used herein. An antigen need not be encoded solely by a full-length nucleotide sequence of a gene. An antigen can be generated, synthesized or derived from a biological sample including a tissue sample, a tumor sample, a cell, or a biological fluid.

The term “anti-tumor effect” as used herein, refers to a biological effect associated with a decrease in tumor volume, a decrease in the number of tumor cells, a decrease in the number of metastases, decrease in tumor cell proliferation, decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies in the prevention of the occurrence of tumor in the first place.

The term “autoantigen” or “self-antigen” refers to an antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from a subject which is subsequently re-introduced into the same subject.

The term “allogeneic” is used to describe a graft derived from a different subject of the same species. As an example, a donor subject may be related or unrelated to the recipient subject, but the donor subject has immune system markers which are similar to the recipient subject.

The term “xenogeneic” is used to describe a graft derived from a subject of a different species. As an example, the donor subject is from a different species than a recipient subject and the donor subject and the recipient subject can be genetically and immunologically incompatible.

The term “cancer” is used to refer to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

Cancers that may be treated include tumors that are not vascularized, or not yet substantially vascularized, as well as vascularized tumors. The cancers may include non-solid tumors (such as hematological tumors, for example, leukemias and lymphomas) or may include solid tumors. Types of cancers to be treated with the CARs of the disclosure include, but are not limited to, carcinoma, blastoma, and sarcoma, and certain leukemia or lymphoid malignancies, benign and malignant tumors, and malignancies, e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastases).

A solid tumor antigen is an antigen expressed on a solid tumor. In embodiments, solid tumor antigens are also expressed at low levels on healthy tissue. Examples of solid tumor antigens and their related disease tumors are provided in Table 1.

TABLE 1

| Solid Tumor antigen | Disease tumor |
|---|---|
| PRLR | Breast cancer |
| CLCA1 | colorectal cancer |
| MUC12 | colorectal cancer |
| GUCY2C | colorectal cancer |
| GPR35 | colorectal cancer |
| CR1L | Gastric cancer |
| MUC 17 | Gastric cancer |
| TMPRSS11B | esophageal cancer |
| MUC21 | esophageal cancer |
| TMPRSS11E | esophageal cancer |
| CD207 | bladder cancer |
| SLC30A8 | pancreatic cancer |
| CFC1 | pancreatic cancer |
| SLC12A3 | Cervical cancer |
| SSTR1 | Cervical tumor |
| GPR27 | Ovary tumor |
| FZD10 | Ovary tumor |
| TSHR | Thyroid Tumor |
| SIGLEC15 | Urothelial cancer |
| SLC6A3 | Renal cancer |
| KISS1R | Renal cancer |
| QRFPR | Renal cancer: |
| GPR119 | Pancreatic cancer |
| CLDN6 | Endometrial cancer/Urothelial |
| | cancer |
| UPK2 | Urothelial cancer (including |
| | bladder cancer) |
| ADAM12 | Breast cancer, pancreatic cancer, |
| | and the like |
| SLC45A3 | Prostate cancer |
| ACPP | Prostate cancer |
| MUC21 | Esophageal cancer |
| MUC16 | Ovarian cancer |
| MS4A12 | Colorectal cancer |
| ALPP | Endometrial cancer |
| CEA | Colorectal carcinoma |
| EphA2 | Glioma |
| FAP | Mesothelioma |
| GPC3 | Lung squamous cell carcinoma |
| IL-13-Rα2 (IL-13 receptor | Glioma |
| alpha 2) | |
| Mesothelin | Metastatic cancer |
| PSMA | Prostate cancer |
| ROR1 | Breast lung carcinoma |
| VEGFR-II | Metastatic cancer |
| GD2 | Neuroblastoma |
| FR-α | Ovarian carcinoma |
| ErbB2 | Carcinoma |
| EpCAM | Carcinoma |
| EGFRvIII | Glioma-Glioblastoma |
| EGFR | Glioma-NSCL cancer |
| tMUC1 | Cholangiocarcinoma, Pancreatic |
| | cancer, |
| PSCA | pancreas, stomach, or prostate |
| | cancer |

Throughout this specification, unless the context requires otherwise, the words “comprise,” “includes” and “including” will be understood to imply the inclusion of a stated step or element (ingredient or component) or group of steps or elements (ingredients or components) but not the exclusion of any other step or element or group of steps or elements.

The phrase “consisting of” is meant to include, and is limited to, whatever follows the phrase “consisting of.” Thus, the phrase “consisting of” indicates that the listed elements or steps are required or mandatory and that no other elements may be present.

The phrase “consisting essentially of” is meant to include any element listed after the phrase and can include other elements or steps that do not interfere with or contribute to the activity or action specified in the disclosure for the listed elements or steps. Thus, the phrase “consisting essentially of” indicates that the listed elements or steps are required or mandatory, but that other elements or steps are optional and may or may not be present depending upon whether or not they affect the activity or action of the listed elements or steps. In embodiments, those elements or steps that do not affect an embodiment are those elements or steps that do not alter the embodiment's ability in a statistically significant manner to perform a function in vitro or in vivo, such as killing cancer cells in vitro or in vivo.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

The term “co-stimulatory ligand” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including at least one of proliferation, activation, differentiation, and other cellular responses. A co-stimulatory ligand can include B7-1 (CD80), B7-2 (CD86), PD-L1, PD-L2, 4-1 BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, a ligand for CD7, an agonist or antibody that binds the Toll ligand receptor and a ligand that specifically binds B7-H3. A co-stimulatory ligand also includes, inter alia, an agonist or an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

The term “co-stimulatory signal” refers to a signal, which in combination with a primary signal, such as TCR/CD3 ligation, leads to T cell proliferation and/or upregulation or downregulation of key molecules.

The terms “co-stimulatory signaling region”, “co-stimulatory domain”, and “co-stimulation domain” are used interchangeably to refer to one or more additional stimulatory domain in addition to a stimulatory or signaling domain such as CD3 zeta. The terms “stimulatory” or “signaling” domain (or region) are also used interchangeably, when referring, for example, to CD3 zeta, the primary signaling domain. In embodiments, the co-stimulatory signaling domain and the stimulatory signaling domain can be on the same molecule or different molecules in the same cell.

The terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. The term “disease” is a state of health of a subject wherein the subject cannot maintain homeostasis, and wherein if the disease is not ameliorated then the subject's health continues to deteriorate. In contrast, a “disorder” in a subject is a state of health in which the animal is able to maintain homeostasis, but in which the animal's state of health is less favorable than it would be in the absence of the disorder. Left untreated, a disorder does not necessarily cause a further decrease in the animal's state of health.

The term “effective” refers to adequate to accomplish a desired, expected, or intended result. For example, an “effective amount” in the context of treatment may be an amount of a compound sufficient to produce a therapeutic or prophylactic benefit.

The term “encoding” refers to the inherent property of specific sequences of nucleotides in a polynucleotide, such as a gene, a cDNA, or an mRNA, to serve as a template for the synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to naturally-occurring polynucleotide or amino acid sequences that may be found in a given wild-type cell or organism. Also, a particular polynucleotide sequence that is isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression” refers to the transcription and/or translation of a particular nucleotide sequence driven by its promoter. The term “overexpression” refers to the production of a gene product in transgenic organisms or cells that exceeds levels of production in normal or non-transformed organisms or cells.

The term “expression vector” refers to a vector including a recombinant polynucleotide including expression control (regulatory) sequences operably linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses (AAV)) that incorporate the recombinant polynucleotide.

The term “homologous” refers to sequence similarity or sequence identity between two polypeptides or between two polynucleotides when a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. A comparison is made when two sequences are aligned to give maximum homology.

The term “immunoglobulin” or “Ig,” refers to a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

The term “isolated” refers to a material that is substantially or essentially free from components that normally accompany it in its native state. The material can be a cell or a macromolecule such as a protein or nucleic acid. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

The term “substantially purified” refers to a material that is substantially free from components that normally associated with it in its native state. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In embodiments, the cells are cultured in vitro. In embodiments, the cells are not cultured in vitro.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. Moreover, the use of lentiviruses enables integration of the genetic information into the host chromosome resulting in stably transduced genetic information. HIV, SIV, and FIV are all examples of lentiviruses. Vectors derived from lentiviruses offer the means to achieve significant levels of gene transfer in vivo.

The term “modulating,” refers to mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

The term “under transcriptional control” refers to a promoter being operably linked to and in the correct location and orientation in relation to a polynucleotide to control the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

The term “parenteral administration” of a composition includes, e.g., subcutaneous (s.c.), intravenous (i.v.), intramuscular (i.m.), intrasternal injection, or infusion techniques.

The terms “patient,” “subject,” “individual,” and the like are used interchangeably herein, and refer to any animal, such as a mammal, for example, a human or any living organism amenable to the methods described herein. In embodiments, the patient, subject, or individual is a human or mammal. In embodiments, the term “subject” is intended to include living organisms in which an immune response can be elicited (e.g., mammals). Examples of subjects include humans, and animals such as dogs, cats, mice, rats, and transgenic species thereof.

A subject in need of treatment or in need thereof includes a subject having a disease, condition, or disorder that needs to be treated. A subject in need thereof also includes a subject that needs treatment for prevention of a disease, condition, or disorder. Accordingly, the subject can also be in need of prevention of a disease condition or disorder. In embodiments, the disease is cancer.

The term “polynucleotide” or “nucleic acid” refers to mRNA, RNA, cRNA, rRNA, cDNA or DNA. The term typically refers to a polymeric form of nucleotides of at least 10 bases in length, either ribonucleotides or deoxynucleotides or a modified form of either type of nucleotide. The term includes all forms of nucleic acids including single and double stranded forms of nucleic acids.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs.

The terms “polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In embodiments, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In embodiments, a polypeptide variant is distinguished from a reference polypeptide by one or more substitutions, which may be conservative or non-conservative. In embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted or replaced with different amino acid residues.

The term “promoter” refers to a DNA sequence recognized by the synthetic machinery of the cell or introduced synthetic machinery, required to initiate the specific transcription of a polynucleotide sequence. The term “expression control (regulatory) sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

“NFAT promoter” refers to one or more NFAT binding sites or motifs linked to a minimal promoter of any gene expressed by T cells. In embodiments, the minimal promoter of a gene expressed by T cells is a minimal human IL-12 promoter. NFAT (nuclear factor of activated T cells) are transcription factors. Examples of NFAT transcription factors include NFAT1, NFAT2, NFAT3, NFAT4, and NFAT5. These transcription factors bind NFAT binding sites or motifs in the NFAT promoter. The NFAT promoter (or a functional portion or functional variant thereof) can comprise any number of binding motifs, e.g., at least two, at least three, at least four, at least five, or at least six, at least seven, at least eight, at least nine, at least ten, at least eleven, or up to twelve binding motifs. In embodiments, the NFAT promoter comprises six NFAT binding motifs. In an especially preferred embodiment, the NFAT promoter nucleotide sequence comprises or consists of a functional portion or functional variant thereof.

The NFAT promoter (or a functional portion or functional variant thereof) is operatively associated with the nucleotide sequence encoding IL-12 (or a functional portion or functional variant thereof). “Operatively associated with” means that the nucleotide sequence encoding IL-12 (or a functional portion or functional variant thereof) is transcribed into IL-12 mRNA when the NFAT protein binds to the NFAT promoter sequence (or a functional portion or functional variant thereof). Without being bound to a particular theory, it is believed that NFAT is regulated by a calcium signaling pathway. In particular, it is believed that TCR stimulation (by, e.g., an antigen) and/or stimulation of the calcium signaling pathway of the cell (by, e.g., PMA/lonomycin) increases intracellular calcium concentration and activates calcium channels. It is believed that the NFAT protein is then dephosporylated by calmoduin and translocates to the nucleus where it binds the NFAT promoter sequence (or a functional portion or functional variant thereof) and activates downstream gene expression. By providing an NFAT promoter (or a functional portion or functional variant thereof) that is operatively associated with the nucleotide sequence encoding IL-12 (or a functional portion or functional variant thereof), the nucleic acids described herein advantageously make it possible to express IL-12 (or a functional portion or functional variant thereof) only when the host cell including the nucleic acid is stimulated by, e.g., PMA/lonomycin and/or an antigen. More information can be found at U.S. Pat. No. 8,556,882, which is incorporated by the reference.

The term “bind,” “binds,” or “interacts with” refers to a molecule recognizing and adhering to a second molecule in a sample or organism but does not substantially recognize or adhere to other structurally unrelated molecules in the sample. The term “specifically binds,” as used herein with respect to an antibody, refers to an antibody which recognizes a specific antigen, but does not substantially recognize or bind other molecules in a sample. For example, an antibody that specifically binds an antigen from one species may also bind that antigen from one or more species. But, such cross-species reactivity does not itself alter the classification of an antibody as being specific. In another example, an antibody that specifically binds an antigen may also bind different allelic forms of the antigen. However, such cross reactivity does not itself alter the classification of an antibody as being specific. In some instances, the terms “specific binding” or “specifically binding,” can be used to describe the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A), in a reaction containing labeled “A” and the antibody, will reduce the amount of labeled A bound to the antibody.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding, and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

Zinc finger binding domains can be “engineered” to bind to a predetermined nucleotide sequence, for example via engineering (altering one or more amino acids) of the recognition helix region of a naturally occurring zinc finger protein. Further, a Zinc finger binding domain may be fused a DNA-cleavage domain to form a Zinc finger nuclease (ZFN) targeting a specific desired DNA sequence. For example, a pair of ZFNs (e.g., a ZFN-left arm and a ZFN-right arm) may be engineered to target and cause modifications of specific desired DNA sequences (e.g., TRAC genes).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In embodiments, fusion polypeptides are used for targeted double-stranded DNA cleavage.

A “target site” or “target sequence” is a nucleic acid sequence that defines a portion of a nucleic acid to which a binding molecule will bind, provided sufficient conditions for binding exist. For example, the sequence 5′ GAATTC 3′ is a target site for the Eco RI restriction endonuclease.

A “fusion” molecule is a molecule in which two or more subunit molecules are linked, preferably covalently. The subunit molecules can be the same chemical type of molecule or can be different chemical types of molecules. Examples of the first type of fusion molecule include, but are not limited to, fusion proteins (for example, a fusion between a ZFP DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage, and polypeptide ligation can also be involved in the expression of the protein in a cell. Methods for polynucleotide and polypeptide delivery to cells are presented elsewhere in this disclosure.

“Modulation” of gene expression refers to a change in the activity of a gene. Modulation of expression can include but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

By “statistically significant,” it is meant that the result was unlikely to have occurred by chance. Statistical significance can be determined by any method known in the art. Commonly used measures of statistical significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less. A “decreased” or “reduced” or “lesser” amount is typically a “statistically significant” or a physiologically significant amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

The term “stimulation,” refers to a primary response induced by binding of a stimulatory molecule (e.g., a TCR/CD3 complex) with its cognate ligand thereby mediating a signal transduction event, such as signal transduction via the TCR/CD3 complex. Stimulation can mediate altered expression of certain molecules, such as downregulation of TGF-β, and/or reorganization of cytoskeletal structures. CD3 zeta is not the only suitable primary signaling domain for a CAR construct with respect to the primary response. For example, back in 1993, both CD3 zeta and FcRy were shown as functional primary signaling domains of CAR molecules. Eshhar et al., “Specific activation and targeting of cytotoxic lymphocytes through chimeric single chains consisting of antibody-binding domains and the gamma or zeta subunits of the immunoglobulin and T cell receptors” PNAS, 1993 Jan. 15; 90(2):720-4, showed that two CAR constructs in which an scFv was fused to “either the FcR gamma chain or the CD3 complex chain” triggered T cell activation and target cell. Notably, as demonstrated in Eshhar et al., CAR constructs containing only the primary signaling domain CD3 zeta or FcR gamma are functional without the co-presence of co-stimulatory domains. Additional non-CD3 zeta based CAR constructs have been developed over the years. For example, Wang et al. (,“A Chimeric Antigen Receptor (CARs) Based Upon a Killer Immunoglobulin-Like Receptor (KIR) Triggers Robust Cytotoxic Activity in Solid Tumors” Molecular Therapy, vol. 22, no. Suppl.1, May 2014, page S57) tested a CAR molecule in which an scFv was fused to “the transmembrane and cytoplasmic domain of” a killer immunoglobulin-like receptor (KIR). Wang et al. reported that, “a KIR-based CAR targeting mesothelin (SS 1-KIR) triggers antigen-specific cytotoxic activity and cytokine production that is comparable to CD3˜-based CARs.” A second publication from the same group, Wang et al. (“Generation of Potent T-cell Immunotherapy for Cancer Using DAP12-Based, Multichain, Chimeric Immunoreceptors” Cancer Immunol Res. 2015 July; 3(7):815-26) showed that a CAR molecule in which “a single-chain variable fragment for antigen recognition was fused to the transmembrane and cytoplasmic domains of KIR2DS2, a stimulatory killer immunoglobulin-like receptor (KIR)” functioned both in vitro and in vivo “when introduced into human T cells with DAP12, an immunotyrosine-based activation motifs-containing adaptor.”

The term “stimulatory molecule” refers to a molecule on a T cell that specifically binds a cognate stimulatory ligand present on an antigen presenting cell. For example, a functional signaling domain derived from a stimulatory molecule is the zeta chain associated with the T cell receptor complex. The stimulatory molecule includes a domain responsible for signal transduction.

The term “stimulatory ligand” refers to a ligand that when present on an antigen presenting cell (e.g., an APC, a dendritic cell, a B-cell, and the like.) can specifically bind with a cognate binding partner (referred to herein as a “stimulatory molecule”) on a cell, for example a T cell, thereby mediating a primary response by the T cell, including activation, initiation of an immune response, proliferation, and similar processes. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “therapeutic” refers to a treatment and/or prophylaxis. A therapeutic effect is obtained by suppression, remission, or eradication of a disease state or alleviating the symptoms of a disease state.

The term “therapeutically effective amount” refers to the amount of the subject compound that will elicit the biological or medical response of a tissue, system, or subject that is being sought by the researcher, veterinarian, medical doctor or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease and its severity and the age, weight, etc., of the subject to be treated.

The term “treat a disease” refers to the reduction of the frequency or severity of at least one sign or symptom of a disease or disorder experienced by a subject.

The term “transfected” or “transformed” or “transduced” refers to a process by which an exogenous nucleic acid is transferred or introduced into the host cell. A “transfected” or “transformed” or “transduced” cell is one which has been transfected, transformed, or transduced with an exogenous nucleic acid. The cell includes the primary subject cell and its progeny.

The term “vector” refers to a polynucleotide that comprises an isolated nucleic acid that can be used to deliver the isolated nucleic acid to the interior of a cell. The cell can be an in vitro cell or an in vivo cell in a subject. Numerous vectors are known in the art including linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds which facilitate transfer of nucleic acid into cells, such as, for example, polylysine compounds, liposomes, and the like. Examples of viral vectors include, adenoviral vectors, adeno-associated virus vectors, retroviral vectors, and others. For example, lentiviruses are complex retroviruses, which, in addition to the common retroviral genes gag, pol, and env, contain other genes with regulatory or structural functions. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes, for example, the genes env, vif, vpr, vpu, and nef are deleted making the vector biologically safe.

In embodiments, a polynucleotide encoding the antigen binding molecule and/or therapeutic agent(s) can be used to implement techniques described herein. The method or use includes: providing a viral particle (e.g., AAV, lentivirus or their variants) comprising a vector genome, the vector genome comprising the polynucleotide, wherein the polynucleotide is operably linked to an expression control element conferring transcription of the polynucleotide; and administering an amount of the viral particle to the subject such that the polynucleotide is expressed in the subject. In embodiments, the AAV preparation may include AAV vector particles, empty capsids and host cell impurities, thereby providing an AAV product substantially free of AAV empty capsids. More information of the administration and preparation of the viral particle may be found at the U.S. Pat. No. 9,840,719 and Milani et al., Sci. Transl. Med. 11, eaav7325 (2019) 22 May 2019, which are incorporated herein by reference. In embodiments, the polynucleotide may integrate into the genome of the modified cell and the progeny of the modified cell will also express the polynucleotide, resulting in a stably transfected modified cell. In embodiments, the modified cell expresses the polynucleotide encoding the CAR but the polynucleotide does not integrate into the genome of the modified cell such that the modified cell expresses the transiently transfected polynucleotide for a finite period of time (e.g., several days), after which the polynucleotide is lost through cell division or other factors. For example, the polynucleotide is present in the modified cell in a recombinant DNA construct, in an mRNA, or in a viral vector, and/or the polynucleotide is an mRNA, which is not integrated into the genome of the modified cell. In embodiments, the vector is a lentivirus. In embodiments, the lentivirus can be packaged with a particle (e.g., a Nano particle) such as to be released for a predetermined time and directly infused to the subject such that the lentivirus can be transferred to T cells of the subject.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The T cell response in a subject refers to cell-mediated immunity associated with a helper, killer, regulatory, and other types of T cells. For example, T cell response may include activities such as assistance to other white blood cells in immunologic processes and identifying and destroying virus-infected cells and tumor cells. T cell response in the subject may be measured via various indicators such as the number of virus-infected cells and/or tumor cells that T cells kill, an amount of cytokines that T cells release, for example, in co-culturing with virus-infected cells and/or tumor cells, a level of proliferation of T cells in the subject, a phenotype change of T cells (e.g., changes to memory T cells), and the longevity or lifespan of T cells in the subject.

In embodiments, in vitro killing assay may be performed by measuring the killing efficacy of CAR T cells by co-culturing CAR T cells with antigen-positive cells. CAR T cells may be considered to have killing effect on the corresponding antigen-positive cells by showing a decrease in the number of corresponding antigen-positive cells co-cultured with CAR T cells and an increase in the release of cytokines such as IFN-γ, TNF-α, and the like, as compared to control cells that do not express the corresponding antigen. Further, in vivo antitumor activity of the CAR T cells may be tested. For example, xenograft models can be established using the antigens described herein in immunodeficient mice. Heterotransplantation of human cancer cells or tumor biopsies into immunodeficient rodents (xenograft models) has, for the past two decades, constituted the major preclinical screen for the development of novel cancer therapeutics (Song et al., Cancer Res. PMC 2014 Aug. 21, and Morton et al., Nature Protocols, 2, -247-250 (2007)). To evaluate the anti-tumor activity of CAR T cells in vivo, immunodeficient mice bearing tumor xenografts were evaluated for CAR T cell anti-tumor activity, for example, a decrease in mouse tumors and/or mouse blood cytokines, such as IFN-γ, TNF-α, and the like.

The term “chimeric antigen receptor” or alternatively a “CAR” refers to a recombinant polypeptide comprising at least an extracellular antigen binding domain, a transmembrane domain, and an intracellular domain (e.g., cytoplasmic domain) including an intracellular signaling domain. In embodiments, the domains in the CAR polypeptide are on the same polypeptide chain, for example, comprising a chimeric fusion protein. In embodiments, the domains of the CAR polypeptide are not on the same molecule, for example, not contiguous with each other, or are on different polypeptide chains.

In embodiments, the intracellular signaling domain may include a functional signaling domain derived from a stimulatory molecule and/or a co-stimulatory molecule as described herein. In embodiments, the intracellular signaling domain includes a functional signaling domain derived from a primary signaling domain (e.g., a primary signaling domain of CD3-zeta). In embodiments, the intracellular signaling domain further includes one or more functional signaling domains derived from at least one co-stimulatory molecule. The co-stimulatory signaling region refers to a portion of the CAR including the intracellular domain of a co-stimulatory molecule. Co-stimulatory molecules can include cell surface molecules for inducing an efficient response from the lymphocytes (in response to an antigen).

Between the extracellular domain and the transmembrane domain of the CAR, there can be incorporated a spacer domain. As used herein, the term “spacer domain” generally means any oligo- or polypeptide that functions to link the transmembrane domain to the extracellular domain and/or the cytoplasmic domain in the polypeptide chain. A spacer domain may include up to 300 amino acids, 10 to 100 amino acids, or 25 to 50 amino acids.

The extracellular domain of a CAR may include an antigen binding domain (e.g., a scFv, a single domain antibody, or TCR, such as a TCR alpha binding domain or a TCR beta binding domain), that targets a specific tumor marker (e.g., a tumor antigen). Tumor antigens are proteins that are produced by tumor cells that elicit an immune response, particularly T cell mediated immune responses. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. For example, when the antigen that the CAR binds is CD19, the CAR thereof is referred to as CD19 CAR. 19CAR, CD19CAR, CD19 CAR, or CD19-CAR), which is a CAR molecule that includes an antigen binding domain that binds CD19.

In embodiments, the extracellular ligand-binding domain comprises a scFv comprising the light chain variable (VL) region and the heavy chain variable (VH) region of a target antigen-specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)₃ (SEQ ID: 2), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides comprising about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

In embodiments, the tumor antigen includes HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor α 2, IL-11 receptor α, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, TEM8, or viral-associated antigens expressed by a tumor. In embodiments, the binding element of the CAR includes any antigen binding moiety that when bound to its cognate antigen, affects a tumor cell such that the tumor cell fails to grow, decrease in size, or dies.

The CAR can be a bispecific CAR. For example, the two antigen binding domains are on the same CAR (a bispecific CAR or tandem CAR (tanCAR)), on different CAR molecules, or on a CAR and T cell receptor (TCR). A single CAR can include two different antigen binding domains, or the two different antigen binding domains are each on a separate CAR. The CAR can have more than two antigen binding domains, for example, a multispecific CAR. The antigen binding domains of the multispecific CAR can be on the same CAR or on separate CAR, such as one antigen binding domain on each CAR.

In embodiments, the intracellular domain of the CAR comprises a co-stimulatory signaling region that comprises an intracellular domain of a co-stimulatory molecule selected from the group consisting of CD27, CD28, 4-1 BB, OX40, CD30, CD40, PD-1, ICOS, lymphocyte function-associated antigen-1 (LFA-1), CD2, CD7, LIGHT, NKG2C, B7-H3, and any combination thereof.

In embodiments, the intracellular domain comprises a CD3 zeta signaling domain. Embodiments relate to a vector comprising the isolated nucleic acid sequence described herein. Embodiments relate to an isolated cell comprising the isolated nucleic acid sequence described herein.

The cells, including CAR cells and modified cells, described herein can be derived from a stem cell. The stem cells may be adult stem cells, embryonic stem cells, or non-human stem cells, cord blood stem cells, progenitor cells, bone marrow stem cells, induced pluripotent stem cells, totipotent stem cells, or hematopoietic stem cells. The cells can also be a dendritic cell, a NK-cell, a B-cell, or a T cell selected from the group consisting of inflammatory T lymphocytes, cytotoxic T lymphocytes, regulatory T lymphocytes, and helper T lymphocytes. In embodiments, the cells can be derived from the group consisting of CD4+ T-lymphocytes and CD8+ T-lymphocytes. Prior to expansion and genetic modification of the cells described herein, a source of cells may be obtained from a subject through a variety of non-limiting methods. T cells may be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In embodiments, any number of T cell lines available and known to those skilled in the art, can be used. In embodiments, the cells may be derived from a healthy donor, from a patient diagnosed with cancer, or from a patient diagnosed with an infection. In embodiments, the cells are part of a mixed population of cells which present different phenotypic characteristics.

A population of cells refers to a group of two or more cells. The cells of the population could be the same, such that the population is a homogenous population of cells. The cells of the population could be different, such that the population is a mixed population or a heterogeneous population of cells. For example, a mixed population of cells could include modified cells comprising a first CAR and cells comprising a second CAR, wherein the first CAR and the second CAR bind different antigens.

The term “stem cell” refers to any type of cell which has the capacity for self-renewal and the ability to differentiate into other kind(s) of cell. For example, a stem cell gives rise either to two daughter stem cells (as occurs in vitro with embryonic stem cells in culture) or to one stem cell and a cell that undergoes differentiation (as occurs e.g. in hematopoietic stem cells, which give rise to blood cells). Different categories of stem cells may be distinguished on the basis of their origin and/or on the extent of their capacity for differentiation into other types of cell. Stem cells can include embryonic stem (ES) cells (i.e., pluripotent stem cells), somatic stem cells, induced pluripotent stem cells, and any other types of stem cells.

Pluripotent embryonic stem cells can be found in the inner cell mass of a blastocyst and have high innate capacity for differentiation. For example, pluripotent embryonic stem cells have the potential to form any type of cell in the body. When grown in vitro for long periods of time, ES cells maintain pluripotency, and progeny cells retain the potential for multilineage differentiation.

Somatic stem cells can include fetal stem cells (from the fetus) and adult stem cells (found in various tissues, such as bone marrow). These cells have been regarded as having a capacity for differentiation lower than that of the pluripotent ES cells—with the capacity of fetal stem cells being greater than that of adult stem cells; they apparently differentiate into only a limited number of different types of cells and have been described as multipotent. “Tissue-specific” stem cells normally give rise to only one type of cell. For example, embryonic stem cells can differentiate into blood stem cells (e.g., Hematopoietic stem cells (HSCs)), which can further differentiate into various blood cells (e.g., red blood cells, platelets, white blood cells, etc.).

Induced pluripotent stem cells (iPS cells or iPSCs) can include a type of pluripotent stem cell artificially derived from a non-pluripotent cell (e.g., an adult somatic cell) by inducing expression of specific genes. Induced pluripotent stem cells are similar to naturally occurring pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability. Induced pluripotent cells can be isolated from adult stomach, liver, skin, and blood cells.

In embodiments, the CAR cells, the modified cell, or the cell is a T cell, a NK cell, a macrophage or a dendritic cell. For example, the CAR cells, the modified cell, or the cell is a T cell.

T cells, or T lymphocytes, are a type of white blood cell of the immune system. There are various types of T cells including T helper (TH) cells, cytotoxic T (TC) cells (T killer cells, killer T cells), natural killer T (NKT) cells, memory T (Tm) cells, regulatory T (Treg) cells, and gamma delta T (γδ T) cells.

T helper (TH) cells assist other lymphocytes, for example, activating cytotoxic T cells and macrophages and maturation of B cells into plasma cells and memory B cells. These T helper cells express CD4 glycoprotein on their surface and are also known as CD4+ T cells. Once activated, these T cells divide rapidly and secrete cytokines.

Cytotoxic T (TC) cells destroy virus-infected cells and tumor cells and are also involved in transplant rejection. They express CD8 protein on their surface. Cytotoxic T cell release cytokines.

Natural Killer T (NKT) cells are different from natural killer cells. NKT cells recognize glycolipid antigens presented by CD1d. Once activated, NKT cells produce cytokine and release cell killing molecules.

Memory T (Tm) cells are long-lived and can expand to large number of effector T cells upon re-exposure to their cognate antigen. Tm cells provide the immune system with memory against previously encountered pathogens. There are various subtypes of Tm cells including central memory T (TCM) cells, effector memory T (TEM) cells, tissue resident memory T (TRM) cells, and virtual memory T cells. Tm cells are either CD4+ or CD8+ and usually CD45RO.

Regulatory T (Treg) cells shut down T cell mediated immunity at the end of an immune reaction and suppress autoreactive T cells that escaped the process of negative selection in the thymus. Subsets of Treg cells include thymic Treg and peripherally derived Treg. Both subsets of Treg require the expression of the transcription factor FOXP3.

Gamma delta T (γδ T) cells are a subset of T cells that possess a γδ T cell receptor (TCR) on the cell surface, as most T cells express the as TCR chains. γδ T cells are less common in human and mice and are mainly found in the gut mucosa, skin, lung, and uterus. They are involved in the initiation and propagation of immune responses.

In embodiments, the antigen binding molecule is a T Cell Receptor (TCR). In embodiments, the TCR is modified TCR. In embodiments, the TCR is derived from spontaneously occurring tumor-specific T cells in patients. In embodiments, the TCR binds a tumor antigen. In embodiments, the tumor antigen comprises CEA, gp100, MART-1, p53, MAGE-A3, or NY-ESO-1. In embodiments, the TCR comprises TCRγ and TCRδ chains or TCRα and TCRβ chains.

In embodiments, a T cell clone that expresses a TCR with high affinity for the target antigen may be isolated. In embodiments, tumor-infiltrating lymphocytes (TILs) or peripheral blood mononuclear cells (PBMCs) may be cultured in the presence of antigen-presenting cells (APCs) pulsed with a peptide representing an epitope known to elicit a dominant T cell response when presented in the context of a defined HLA allele. High-affinity clones may be then selected on the basis of MHC-peptide tetramer staining and/or the ability to recognize and lyse target cells pulsed with low titrated concentrations of cognate peptide antigen. After the clone has been selected, the TCRα and TCRβ chains or TCRγ and TCRδ chains are identified and isolated by molecular cloning. For example, for TCRα and TCRβ chains, the TCRα and TCRβ gene sequences are then used to generate an expression construct that ideally promotes stable, high-level expression of both TCR chains in human T cells. The transduction vehicle (e.g., a gammaretrovirus or lentivirus) may be then generated and tested for functionality (antigen specificity and functional avidity) and used to produce a clinical lot of the vector. An aliquot of the final product is then used to transduce the target T cell population (generally purified from patient PBMCs), which is expanded before infusion into the subject.

In embodiments, the APCs include dendritic cells, macrophages, Langerhans cells and B cells, or T cells.

In embodiments, the binding element of the CAR may include any antigen binding moiety that when bound to its cognate antigen, affects a tumor cell for example, it kills the tumor cell, inhibits the growth of the tumor cell, or promotes death of the tumor cell.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the nucleic acid of interest can be produced synthetically, rather than cloned.

The embodiments of the present disclosure further relate to vectors in which a nucleic acid described herein is inserted. Vectors can be derived from retroviruses such as the lentivirus that are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Viruses can be used to deliver nucleic acids into a cell in vitro and in vivo (in a subject). Examples of viruses useful for delivery of nucleic acids into cells include retrovirus, adenovirus, herpes simplex virus, vaccinia virus, and adeno-associated virus.

There also exist non-viral methods for deliverying nucleic acids into a cell, for example, electroporation, gene gun, sonoporation, magnetofection, and the use of oligonucleotides, lipoplexes, dendrimers, and inorganic nanoparticles.

The expression of natural or synthetic nucleic acids encoding CARs is typically achieved by operably linking a nucleic acid encoding the CAR polypeptide or portions thereof to one or more promoters and incorporating the construct into an expression vector. The vectors can be suitable for replication and integration into eukaryotes. Typical cloning vectors contain transcription and translation terminators, initiation sequences, and promoters useful for regulation of the expression of the desired nucleic acid sequence.

Additional information related to expression of synthetic nucleic acids encoding CARs and gene transfer into mammalian cells is provided in U.S. Pat. No. 8,906,682, incorporated by reference in its entirety.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, “therapeutic amount”, or “effective amount” is indicated, the precise amount of the compositions of the present disclosure to be administered can be determined by a physician with consideration of individual differences in age, weight, tumor size, extent of infection or metastasis, and condition of the patient (subject). It can be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions can also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art by monitoring the patient for signs of disease and adjusting the treatment accordingly. In embodiments, activated T cells are administered to a subject and then subsequently blood is redrawn (or have apheresis performed). T cells are collected, expanded, and reinfused into the subject. This process can be carried out multiple times every few weeks. In embodiments, T cells can be activated from blood draws of from 10 cc to 400 cc. In embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocols, certain populations of T cells can be selected.

The administration of the pharmaceutical compositions described herein can be carried out in any convenient manner, including by aerosol inhalation, injection, ingestion, transfusion, implantation or transplantation. The pharmaceutical compositions described herein can be administered to a patient subcutaneously, intradermally, intratumorally, intranodally, intramedullary, intramuscularly, intravenously (i. v.), or intraperitoneally. In embodiments, the T cell compositions of the present disclosure are administered to a patient by intradermal or subcutaneous injection. In embodiments, the T cell compositions of the present disclosure are administered by i.v. injection. The compositions of T cells may be injected directly into a tumor, lymph node, or site of infection. In embodiments of the present disclosure, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the present disclosure may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In embodiments, the cell compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In embodiments, the cell compositions of the present disclosure are administered following B-cell ablative therapy such as agents that react with CD20, e.g., Rituxan®. For example, subjects may undergo standard treatment with high dose chemotherapy followed by peripheral blood stem cell transplantation. In embodiments, following the transplant, subjects receive an infusion of the expanded immune cells of the present disclosure. In embodiments, expanded cells are administered before or following surgery.

The dosage of the above treatments to be administered to a patient will vary with the precise nature of the condition being treated and the recipient of the treatment. The scaling of dosages for human administration can be performed according to art-accepted practices by a physician depending on various factors.

Additional information on the methods of cancer treatment using engineered or modified T cells is provided in U.S. Pat. No. 8,906,682, incorporated by reference in its entirety.

In embodiments, the population of cells described herein is used in autologous CAR T cell therapy. In embodiments, the CAR T cell therapy is allogenic CAR T cell therapy, TCR T cell therapy, and NK cell therapy.

Embodiments relate to an in vitro method for preparing modified cells. The method may include obtaining a sample of cells from the subject. For example, the sample may include T cells or T cell progenitors. The method may further include transfecting the cells with a DNA encoding at least a CAR, culturing the population of CAR cells ex vivo in a medium that selectively enhances proliferation of CAR-expressing T cells.

In embodiments, the sample is a cryopreserved sample. In embodiments, the sample of cells is from umbilical cord blood or a peripheral blood sample from the subject. In embodiments, the sample of cells is obtained by apheresis or venipuncture. In embodiments, the sample of cells is a subpopulation of T cells.

As used herein, the term “gene fusion” refers to the fusion of at least a portion of a gene to at least a portion of an additional gene. The gene fusion need not include entire genes or exons of genes. In some instances, gene fusion is associated with alternations in cancer. A gene fusion product refers to a chimeric genomic DNA, a chimeric messenger RNA, a truncated protein or a chimeric protein resulting from a gene fusion. The gene fusion product may be detected by various methods described in U.S. Pat. No. 9,938,582, which is incorporated as a reference herein. A “gene fusion antigen” refers to a truncated protein or a chimeric protein that results from a gene fusion. In embodiments, an epitope of a gene fusion antigen may include a part of the gene fusion antigen or an immunogenic part of another antigen caused by the gene fusion. In embodiments, the gene fusion antigen interacts with, or is part of, cell membranes.

In embodiments, detection of mRNA and protein expression levels of a target molecules (e.g., CARs and cytokines) in cells, such as human cells, may be performed using experimental methods such as qPCR and FACS. Further, target molecules specifically expressed in the corresponding tumor cells with very low expression or undetectable expression in normal tissue cells may be identified.

In embodiments, In Vitro Killer Assay as well as killing experiment of CAR T Cells Co-Cultured with Antigen-Positive Cells can be performed. CAR T cells can exhibit a killing effect on the corresponding antigen-positive cells, a decrease in the number of corresponding antigen-positive cells co-cultured with CAR T cells, and an increase in the release of IFN-γ, TNF-α, etc. as compared to control cells that did not express the corresponding antigen.

In embodiments, In Vivo Killer Assay can be performed. For example, mice may be transplanted with corresponding antigen tumor cells, and tumorigenic, transfusion of CAR T cells, and a decrease in mouse tumors and mouse blood IFN-γ, TNF-α, and other signals can be defected.

Embodiments relate to a method of eliciting and/or enhancing T cell response in a subject having a solid tumor or treating a solid tumor in the subject, the method comprising administering an effective amount of T cells comprising the CAR described herein. In embodiments, the intracellular domain of the CAR comprises a co-stimulatory signaling region that comprises an intracellular domain of a co-stimulatory molecule selected from the group consisting of CD27, CD28, 4-11BB, OX40, CD30, CD40, PD-1, ICOS, lymphocyte function-associated antigen-1 (LFA-1), CD2, CD7, LIGHT, NKG2C, B7-H3, and any combination thereof. In embodiments, the intracellular domain comprises a CD3 zeta signaling domain.

Embodiments relate to a vector comprising the isolated nucleic acid described herein.

Embodiments relate to an isolated cell comprising the isolated nucleic acid sequence described herein. Embodiments relate to a composition comprising a population of T cells comprising the CAR described herein. Embodiments relate to a CAR encoded by the isolated nucleic acid sequence described herein. Embodiments relate to a method of eliciting and/or enhancing T cell response in a subject or treating a tumor of the subject, the method comprising: administering an effective amount of T cell comprising the CAR described herein.

In embodiments, the CAR molecules described herein comprise one or more complementarity-determining regions (CDRs) for binding an antigen of interest. CDRs are part of the variable domains in immunoglobulins and T cell receptors for binding a specific antigen. There are three CDRs for each variable domain. Since there is a variable heavy domain and a variable light domain, there are six CDRs for binding an antigen. Further since an antibody has two heavy chains and two light chains, an antibody can have twelve CDRs altogether for binding antigens.

In embodiments, the modified cells described herein includes a CAR molecule comprising at least two different antigen binding domains. The CAR molecule can be a bispecific CAR molecule. For example, the two antigen binding domains can be on the same CAR molecule, on different CAR molecules, or on a CAR molecule and T cell receptor (TCR). A single CAR can include at least two different antigen binding domains, or the two different antigen binding domains are each on a separate CAR molecule. The at least two different antigen binding domains can be on the same CAR molecule or different CAR molecules, but in the same modified cell. Moreover, the at least two different antigen binding domains can be on a CAR molecule and a T cell receptor in the same modified cell. In embodiments, the bispecific CAR molecule can include a binding domain binding an antigen of WBC (e.g., CD19) and a binding domain binding a solid tumor antigen. In embodiments, the bispecific CAR molecule may include two binding domains binding two different solid tumor antigens.

In embodiments, the at least two different antigen binding domains are on different CAR molecules which are expressed by different modified cells. Further, the one or more different antigen binding domains are on a CAR molecule and a T cell receptor, which are expressed by different modified cells.

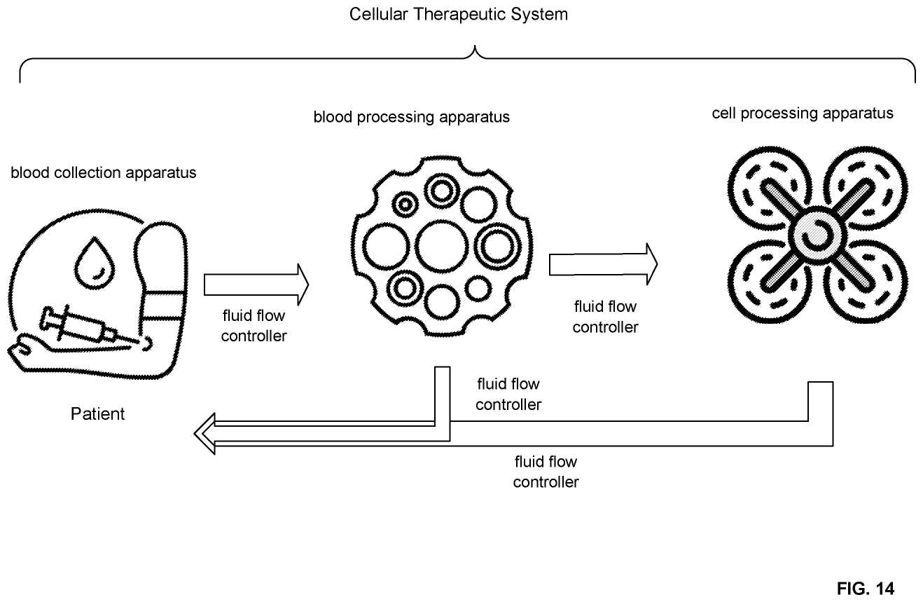

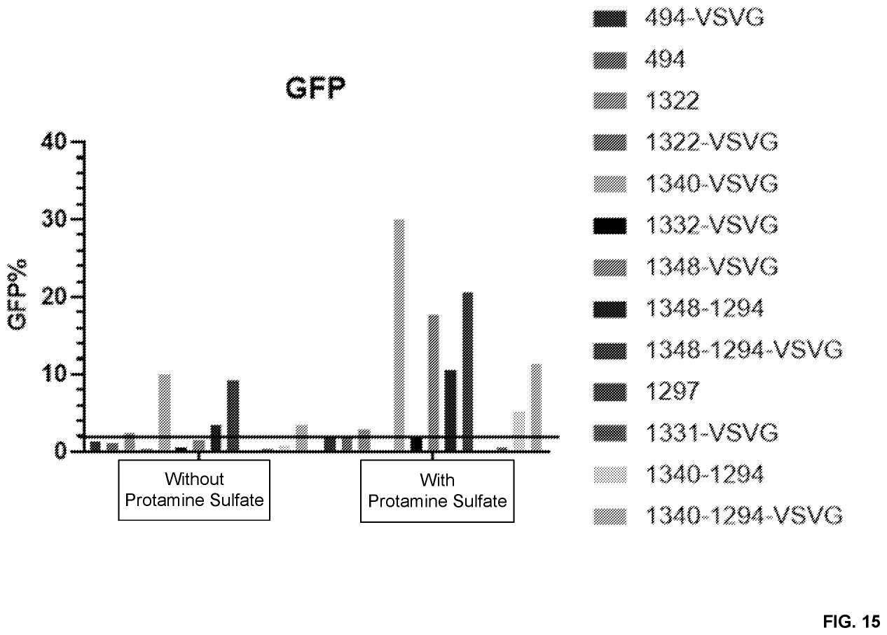

Embodiments relate to a system for immune therapy, the system comprising: a sample or blood processing module configured to obtain CD3+ cells from a blood sample of a subject, for example, a blood collection apparatus (FIG. 14); a cell incubation module configured to activate CD3+ cell and introduce a vector into the CD3+ cells, for example, a blood processing apparatus (FIG. 14), and a cell infusion module configured to infuse at least a portion of the CD3+ cells into the subject, for example, a cell processing apparatus (FIG. 14).

The sample or blood processing module can further comprise a unit configured to obtain cells from the peripheral blood of a subject, for example a human patient. For example, the unit is an apheresis device in which the blood of a person is passed through an apparatus that separates out one particular blood component and returns the remainder of the blood into the systemic circulation of the subject.

In embodiments, the sample separation module can purify CD3+ cells by negative selection using, for example, the RosetteSep T cell enrichment Cocktail or positive selection using anti-CD3 coupled to magnetic beads. Following isolation, CD3+ cells were cultured in X-VIVO 15 (Cambrex, Walkersville, MD) supplemented with 5% normal human AB serum (Valley Biomedical, Winchester, VA), 2 mM L-glutamine (Cambrex), 20 mM HEPES (Cambrex), and IL2 (100 units/mL; R&D Systems). In embodiments, the blood sample can be diluted using Dulbecco phosphate buffered saline (DPBS), and apheresis density gradient centrifugation can be performed using the sample separation module to obtain PBMCs containing lymphocytes. MACS buffer can be used to rinse the PBMCs, and Pan T Cell-Ab cocktail can be mixed with PBMCs and incubated for 5 min. Pan T cell beads cocktail can be added and incubated at 2-8 degrees for 10 min. In embodiments, the sample separation module can include or be coupled to LS columns used to collect CD3+ cells. In embodiments, the output port can be configured to infuse the remaining components of the blood sample or non-desired cells (e.g., CD3− cells) back to the subject and to flow the desired cells (CD3+ cells) to the cell incubation module.

In embodiments, the systems described herein comprise one or more modules, apparatuses and/or devices. In embodiments, the modules can comprise one or more apparatuses and/or devices.

As used herein, the term “flow” or “flowing” with respect to the cells, components of the blood, and/or fluids, refers to moving or transporting them from one location to another.

The cell incubation module can be configured for stimulation/activation and culturing of the CD3+ cells. For example, the CD3+ cells can be stimulated/activated with magnetic beads precoated with agonist antibodies against CD3 and CD28 at a ratio of three beads per cell, and then resuspended at a concentration of 10⁶ CD+3 cells/mL for expansion for a predetermined time (e.g., 2-20 hours). The stimulated/activated CD3+ cells can be then transduced with vectors including nucleic acids encoding CD19-BBζ CAR (CD19 4-1BB CD3 zeta CAR) hours after the cell stimulation/activation and expansion for a predetermined time, for example, 1-5 hours, or harvested at specific time points for analysis. CD3+ cells can be maintained in culture at a concentration of 0.1 to 1×10⁶ cells/mL by adjusting the concentration based on cell counting by flow cytometry using antibodies to human CD4 and CD8. After completion of cell culture, the magnetic beads can be removed and tested for release criteria specified for T cell phenotype, cell viability (≥70%), concentration (80% CD3+CD45+), and transduction efficiency (≥2%). In embodiments, CD3+ cells can be co-cultured with beads conjugated with CD19 antigen comprising a His-tag, for example, 6-histidines attached to the C-terminus of the CD19 antigen, after removal of beads conjugated with anti-CD3 and anti-CD28 and transduction with lentiviral virus containing a nucleic acid encoding an anti-CD19 CAR, for example, CD19 CAR.

The cell infusion module can comprise an input port, an output port, and a rotating container. The input port can be configured to obtain the CD3+ cells from the cell incubation module; the rotating container can be configured to remove beads, for example, magnetic beads precoated with agonist antibodies against CD3 and CD28 or CD19 beads, and to replace culture media of the CD+3 cells with media suitable for infusion; and the output port can be configured to provide the CD+3 cell for the infusion to the subject.

In embodiments, the sample processing module is coupled or connected to the cell incubation module that is coupled or connected to the cell infusion module such that the CD3+ cells flow through the system starting from the subject and ending back to the subject.

In embodiments, the sample processing module comprises: an input port configured to receive the blood sample from the subject; a sample separation module configured to: separate the CD3+ cells from remaining components of the blood sample, and collect the CD3+ cells; and an output port configured to flow the CD3+ cells to the cell incubation module, and flow the remaining components of the blood sample back into the subject.

In embodiments, the sample separation module is configured to contact the blood sample with a composition comprising a CD3 aggregation reagent comprising CD3 recognizing moiety coupled to a magnetic bead; apply gravity sedimentation for sedimentation of CD3+ cells, and a magnetic field gradient to the blood sample for immobilizing the magnetic bead simultaneously; generate a pellet and a supernatant phase; and recover the desired cells from the pellet. In embodiments, the sample separation module is configured to contact the blood sample with a composition comprising molecules that recognize and specifically bind undesired blood components; simultaneously apply gravity sedimentation for sedimentation of undesired blood components, and a magnetic field gradient to the blood sample for immobilizing the magnetic bead; generate a pellet and a supernatant phase; and recover the desired cells from the supernatant phase.

In embodiments, the blood components can include, for example, plasma, red blood cells, white blood cells, platelets, immune cells, proteins such as antibodies and clotting factors, and vitamins.

In embodiments, the system comprises components that are composed of plastic. Examples of such components include storage bags, tubes and such as inlet and outlet tubes. The plastic can comprise polystyrene, polyvinylchloride, polycarbonate, glass, polyacrylate, polyacrylamide, polymethylmethacrylate (PMMA), polyethylene terephthalate (PET), polytetrafluoroethylene (PTFE), thermoplastic polyurethane (TPU), silicone, polyethylene (PE), polylactide (PLA), polyglycolide (PGA), and copolymers thereof. In embodiments, the system comprises components comprising collagen, chitin, alginate, hyaluronic acid, and hyaluronic acid derivatives, In embodiments, the components of the system comprise ceramics, glass materials, hydroxyapatite (HA), and calcium phosphate. In embodiments, the system comprises compositions comprising one or more of the above-mentioned materials.

In embodiments, the system further comprises one or more sensors configured to monitor the separation of the blood sample obtained from the subject, in particular by detecting the formation of layers of the blood sample, the change of pH value of the blood sample, and/or the change in temperature of the blood sample.

In embodiments, the cell incubation module comprises a rotating container configured to culture cells and/or grow cells. The rotating container is disposable and/or has been sterilized.

Embodiments relate to a method of immune therapy by implementing the system, the method comprising: obtaining peripheral blood from a subject; collecting CD3+ cells from the peripheral blood; returning the remaining components of the peripheral blood to the subject; obtaining vectors comprising a nucleic acid encoding a CAR and one or more agents that activate cells, for example, T cells; mixing the vectors with the obtained CD3+ cells and incubating the mixture for a predetermined time to introduce the nucleic acid into the CD3+ cells; replacing culture media of the transfected CD3+ cells with a solution suitable for infusion into the subject; and infusing at least a portion of the solution containing the transfected CD3+ cells into the subject.

In embodiments, the entire method or process is completed in less than 1, 2, 3, 4, or 5 days.

Embodiments relate to an apparatus for sample or blood processing, the apparatus comprising: an input port configured to receive blood sample from a subject, for example, a blood collection apparatus (FIG. 14); a sample separation module configured to isolate desired cells from the blood sample, for example, a blood processing apparatus (FIG. 14), an output port configured to: flow the isolated desired cells for further processing, and return the rest of the cells and/or remaining components of the blood sample to the subject, for example, a cell process apparatus in FIG. 14).

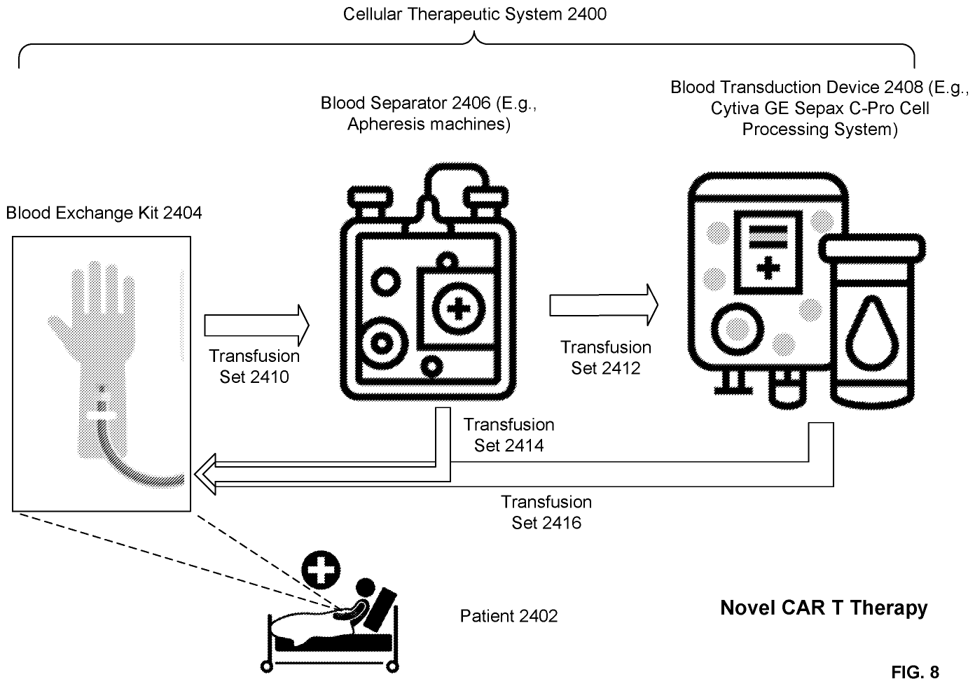

In embodiments, the sample processing apparatus can further comprise a unit configured to obtain cells from the peripheral blood of the subject, such as a human patient, for example, the blood separator 2406 (FIG. 8). In embodiments, the unit is an apheresis device in which the blood of a subject is passed through an apparatus that separates out one particular constituent and returns the remainder to the systemic circulation of the subject.

Embodiments relate to a method for sample processing using the sample processing apparatus, the method comprising: obtaining a blood sample from a subject; isolating desired cells from the blood sample; flowing the desired cells for further processing; returning the remaining components of the blood sample to the subject; introducing a nucleic acid encoding an antigen binding molecule into the desired cells to obtain transfected cells; and administering an effective amount of a composition comprising the transfected cells to a subject in need thereof. In embodiments, the desired cells are CD3+ cells.

Embodiments relate to a method of delivering a therapeutic protein to the peripheral blood system of a subject, the method comprising: administering to the peripheral blood system of the subject, a viral vector, for example, retrovirus, adenovirus, adeno associated virus, and/or Lentivirus vectors, comprising a nucleic acid encoding the therapeutic protein, such that cells, organs, or tissues of the peripheral vascular system of the subject express the therapeutic protein. The therapeutic protein can be an antigen binding molecule.

Embodiments relate to a method of treating cancer in a subject, the method comprising: administering to the peripheral blood system of the subject a viral vector comprising a nucleic acid encoding a CAR molecule such that cells, organs or tissues of the peripheral vascular system of the subject express the CAR molecule; and monitoring cell response, for example, T cell response, resulting from the expression of CAR molecule in the subject.

Embodiments relate to a method of in vivo expression of antigen binding molecule in lymphocytes of a subject, the method comprising: administering to the peripheral blood system of the subject a viral vector comprising a nucleic acid encoding an antigen binding molecule such that cells, organs, or tissues of the peripheral vascular system of the subject express the antigen binding molecule.

Examples of a viral vector includes an adeno associated virus (AAV) vector. AAV is a small nonpathogenic virus of the parvoviridae family. AAV is distinct from other members of this family by its dependence upon a helper virus for replication. In the absence of a helper virus, AAV can integrate in a locus-specific manner into the q arm of chromosome 19. Approximately, 5 kb genome of AAV consists of one segment of single-stranded DNA of either plus or minus polarity. The ends of the genome are short inverted terminal repeats which can fold into hairpin structures and serve as the origin of viral DNA replication. Physically, the parvovirus virion is non-enveloped, and its icosahedral capsid is approximately 20-30 nm in diameter.

The adeno-associated virus (AAV) capsid includes three related proteins, VP1, VP2, and VP3. VP3, the shortest of the three proteins, has a molecular weight of about 59 to 61 kDa and a sequence of about 524 to 544 amino acids. The sequence of VP3 is shared by all three VPs and is known as the VP3 common region. VP2, which is about 57 amino acids longer than VP3, has a molecular weight of about 64 to 67 kDa and a sequence of about 580 to 601 amino acids. The N-terminal region of the VP2 sequence is shared by VP1 and is known as the VP1/VP2 common region. VP1, which is about 137 amino acids longer than VP2, has a molecular weight of about 79 to 82 kDa and a sequence of about 713 to 738 amino acids. The N-terminal region of the VP1 sequence is unique to VP1 and is known as the VP1 unique (VP1u) region. These three capsid proteins, VP1, VP2, and VP3, are typically present in the capsid at a ratio approximating 1:1:10, respectively, although this ratio, particularly the amount of VP3, can vary significantly and should not be considered as limiting in any respect.

The ends of the AAV genome have short inverted terminal repeats (ITR), which have the potential to fold into T-shaped hairpin structures that serve as the origin of viral DNA replication. Within the ITR region, two elements have been described, which are central to the function of the ITR, a GAGC repeat motif and the terminal resolution site (TRS). The GAGC repeat motif has been shown to bind Rep (replication protein) when the ITR is in either a linear or hairpin conformation. This binding serves to position Rep68/78 for cleavage at the TRS, which occurs in a site- and strand-specific manner. In addition to their role in replication, the GAGC repeat motif and the TRS appear to be central to viral integration. Contained within the chromosome 19 integration locus is a Rep binding site with an adjacent TRS. The GAGC repeat motif and the TRS have been shown to be functional for locus-specific integration. AAV are useful as gene therapy vectors as they can penetrate cells and introduce nucleic acid/genetic material so that the nucleic acid/genetic material can be stably maintained in cells.

In addition, these adeno-associated viruses (AAVs) can introduce nucleic acid/genetic material into specific sites, for example, a specific site on chromosome 19. Because AAV is not associated with pathogenic disease in humans, AAV vectors are able to deliver heterologous polynucleotide sequences encoding therapeutic proteins and agents to human patients without causing substantial AAV pathogenesis or disease. Accordingly, recombinant AAV (rAAV) vectors, including serotypes and variants, provide a means for the delivery of nucleic acids encoding proteins into cells ex vivo, in vitro, and in vivo, such that the cells express the encoded proteins. For example, an rAAV vector can include a heterologous nucleic acid encoding a desired protein or peptide. Vector delivery or administration to a subject, such as a mammal, provides the encoded protein to the subject. More Information on gene therapy and its clinical uses can be found at U.S. Pat. Nos. 9,433,688, 9,644,216, and 10,113,183; as well as Milone, M. C., O'Doherty, U. Clinical use of lentiviral vectors, Leukemia 32, 1529-1541 (2018); Rodrigues, G. A., Shalaev, E., Karami, T. K. et al. Pharmaceutical Development of AAV-Based Gene Therapy Products for the Eye. Pharm Res 36, 29 (2019); and Marquez Loza, L. I.; Yuen, E. C.; McCray, P. B., Jr. Lentiviral Vectors for the Treatment and Prevention of Cystic Fibrosis Lung Disease. Genes 2019, 10, 218; which are incorporated by reference in their entirety.

Modified AAV vectors, such as recombinant nucleic acids and polypeptides, are AAV vectors whose sequences have been manipulated, for example engineered or recombined, in a manner that the vector or sequence does not occur in nature. A particular example of a recombinant AAV vector would be one in which a nucleic acid that is not normally present in the wild-type viral genome, such as the AAV, genome, and is inserted within the viral genome and is heterologous to the viral genome. A “recombinant” AAV vector is distinguished from an AAV genome since all, or a part of the viral genome has been replaced with a non-native sequence with respect to the AAV genomic nucleic acid, such as a heterologous nucleic acid sequence. Although the term “recombinant” is not always used herein in reference to AAV vectors, as well as its sequences such as nucleic acids and polypeptides, recombinant forms of AAV, and sequences including nucleic acids and polypeptides, are expressly included in spite of any such omission. Typically for AAV, one or both inverted terminal repeat (ITR) sequences of the AAV genome are retained in the AAV vector. Incorporation of a non-native sequence therefore defines the AAV vector as a “recombinant” AAV (rAAV) vector.

A recombinant AAV vector can be packaged as a “particle” for subsequent transduction of a cell, for example, ex vivo, in vitro, or in vivo transduction. Where a recombinant AAV vector is encapsidated or packaged into an AAV particle, the particle can also be referred to herein as an “rAAV.” Such particles include proteins that encapsidate or package the vector genomes, and in the case of AAV, capsid proteins. An “AAV viral particle” or “AAV particle” refers to a viral particle composed of at least one AAV capsid protein, though typically all three capsid proteins of an AAV, and an encapsidated nucleic acid, referred to as a vector genome. If the particle comprises heterologous nucleic acid, it is typically referred to as “rAAV.”

An AAV vector “genome” refers to the portion of the recombinant plasmid sequence that is ultimately packaged or encapsidated to form an AAV particle. In cases where recombinant plasmids are used to construct or manufacture recombinant AAV vectors, the AAV vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for propagation and recombinant AAV production but is not itself packaged or encapsidated into rAAV particles.

In embodiments, rAAV vectors include capsids derived from AAV1, AAV2, AAV3, AAV4, AAV5, AAV6, AAV7, AAV8, AAV9, AAV10, AAV11, RhlO, Rh74, or AAV-2i8, as well as variants thereof, for example, capsid variants having amino acid insertions, additions and substitutions. AAV vector serotypes and variants include capsid variants, such as LK03, 4-I, and the like. rAAV serotypes and rAAV variants may or may not be distinct from other AAV serotypes (e.g., distinct from VP1, VP2, and/or VP3 sequences). The term “serotype” is a distinction used to refer to an AAV having a capsid that is serologically distinct from other AAV serotypes. Serologic distinctiveness is determined based on the lack of cross-reactivity between antibodies to one AAV as compared to another AAV. Such cross-reactivity differences are usually due to differences in capsid protein sequences/antigenic determinants for example, due to VP1, VP2, and/or VP3 sequence differences of AAV serotypes. Despite the possibility that AAV variants, including capsid variants, may not be serologically distinct from a reference AAV or other AAV serotype, the AAV variants differ by at least one nucleotide or amino acid residue from the reference or other AAV serotype.

Recombinant AAV vector, as well as methods and uses thereof, include any viral strain or serotype. As a non-limiting example, a recombinant AAV vector genome can be based upon any AAV genome, such as AAV-1, -2, -3, -4, -5, -6, -7, -8, 9, -10, -11, -rh74, -rhlO, or AAV-2i8. Such vectors can be based on the same strain or serotype (or subgroup or variant) or be different from each other. As a non-limiting example, a recombinant AAV vector genome based upon one serotype genome can be identical to one or more of the capsid proteins that package the vector. In addition, a recombinant AAV vector genome can be based upon an AAV, for example., AAV2, serotype genome distinct from one or more of the capsid proteins that package the vector, in which case at least one of the three capsid proteins can be from AAV1, AAV2, AAV3, AAV4, AAV5, AAV6, AAV7, AAV8, AAV9, AAV10, AAV11, RhlO, Rh74 or AAV-2i8 or variant, such as capsid variants such as LK03, 4-1, and the like. AAV vectors, therefore, can include nucleic acid and/or protein sequences identical to nucleic acid acnd protein sequences characteristic for a particular serotype.

An “AAV ITR” or “AAV ITRs” refers to the art-recognized regions found at each end of the AAV genome which function together in cis as origins of DNA replication and as packaging signals for the virus. AAV ITRs, together with the AAV rep coding region, provide for the efficient excision and rescue from and integration of a nucleotide sequence interposed between two flanking ITRs into a mammalian cell genome.

The nucleotide sequences of AAV ITRs are known. An “AAV ITR” need not have the wild-type nucleotide sequence depicted, but may be altered, e.g., by the insertion, deletion, or substitution of nucleotides. Additionally, the AAV ITR can be derived from any of several AAV serotypes. Furthermore, 5′ and 3′ ITRs which flank a heterologous nucleic acid sequence in an AAV vector need not necessarily be identical or derived from the same AAV serotype or isolate, so long as they function as intended, for example, to allow for excision and rescue of the sequence of interest from a host cell genome or vector, and to allow integration of the heterologous sequence into the recipient cell genome.

AAV “empty capsids” as used herein do not contain a vector genome (hence, the term “empty”), in contrast to “genome containing capsids,” which contain an AAV vector genome. Empty capsids are virus-like particles in that they react with one or more antibodies that react with the intact virus, such as a genome containing AAV vector. AAV empty capsids are believed to bind to or react with antibodies against the AAV vectors, thereby functioning as a decoy to reduce inactivation of the AAV vector. Such a decoy acts to absorb antibodies directed against the AAV vector, thereby increasing or improving AAV vector transgene transduction of cells (introduction of the transgene), and in turn, increased cellular expression of the transcript and/or encoded protein. Empty capsids can be generated and purified to the desired quality, and their quantities can be determined. For example, empty capsid titer can be measured by spectrophotometry by optical density at 280 nm wavelength (based on Sommer et al., Mol. Ther. 2003 January; 7(1): 122-8). Empty-AAV or empty capsids are sometimes naturally found in AAV vector preparations. Such natural mixtures can be used as described herein or if desired, be manipulated to increase or decrease the amount of empty capsid and/or vector. For example, the amount of empty capsid can optionally be adjusted to an amount that would be expected to reduce the inhibitory effect of antibodies that react with an AAV vector that is intended to be used for vector-mediated gene transduction in the subject. The use of empty capsids is described in US Publication 2014/0336245. In embodiments, AAV empty capsids are formulated with rAAV vectors and/or administered to a subject. In embodiments, AAV empty capsids are formulated with less than or an equal amount of vector, for example, about 1.0 to 100-fold AAV vectors to AAV empty capsids, or about a 1:1 ratio of AAV vectors to AAV empty capsids. In embodiments, AAV vectors are formulated with an excess of AAV empty capsids, such as greater than 1-fold AAV empty capsids to AAV vectors, or 1.0 to 100-fold AAV empty capsids to AAV vectors.

In embodiments, the pair of ITRs are derived or obtained from or comprise the ITR sequences from AAV1, AAV2, AAV3, AAV4, AAV5, AAV6, AAV7, AAV8, AAV9, AAV10, AAV11, AAV 12, LK01, LK02, LK03, AAV 4-1, AAV-2i8 ITRs, or a combination thereof.

In embodiments, the one or more AAV capsid proteins are derived or obtained from or comprise one or more capsid protein sequences from AAV1, AAV2, AAV3, AAV4, AAV5, AAV6, AAV7, AAV8, AAV9, AAV10, AAV11, AAV 12, LK01, LK02, LK03, AAV 4-1, AAV-2i8 ITRs, or a combination thereof.

In embodiments, the rAAV vector further comprises one or more of an intron, an expression control element, a filler polynucleotide sequence and/or poly A signal, or a combination thereof.

rAAV vectors, rAAV particles, and pharmaceutical compositions thereof can be packaged in single or multiple unit dosage forms for ease of administration and uniformity of dosage. A “unit dose” or a “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce the desired effect (e.g., prophylactic or therapeutic effect). Unit dosage forms can be within, for example, ampules and vials, which can include a liquid composition or a composition in a freeze-dried or lyophilized state; a sterile liquid carrier, for example, can be added prior to administration or delivery in vivo. Individual unit dosage forms can be included in unit-dose or multi-dose kits or containers.

In embodiments, the rAAV particles are administered at a dose in a range from about 1×10⁸ to 1×10¹⁰, 1×10¹⁰ to 1×10¹¹, 1×10¹¹ to 1×10¹², 1×10¹² to 1×10¹³, or 1×10¹³ to 1×10⁴ vector genomes per kilogram (vg/kg) bodyweight of the subject. In embodiments, the rAAV particles are administered at a dose of less than 1×10¹⁰ vector genomes per kilogram (vg/kg) of the subject.

In embodiments, the rAAV particles are administered at a dose of about 5×10¹¹ vector genomes per kilogram (vg/kg) of the subject. In embodiments, the rAAV particles administered are at least 1×10¹⁰ vector genomes (vg) per kilogram (vg/kg) of the bodyweight of the subject, or between about 1×10¹⁰ to 1×10¹¹ vg/kg of the bodyweight of the subject, or between about 1×10¹¹ to 1×10¹² vg/kg (e.g., about 1×10¹¹ to 2×10¹¹ vg/kg or about 2×10¹¹ to 3×10¹¹ vg/kg or about 3×10¹¹ to 4×10¹¹ vg/kg or about 4×10¹¹ to 5×10¹¹ vg/kg or about 5×10¹¹ to 6×10¹¹ vg/kg or about 6×10¹¹ to 7×10¹¹ vg/kg or about 7×10¹¹ to 8×10¹¹ vg/kg or about 8×10¹¹ to 9×10¹¹ vg/kg or about 9×10¹¹ to 1×10¹² vg/kg) of the bodyweight of the subject, or between about 1×10¹⁰ to 1×10¹² vg/kg of the bodyweight of the subject, to achieve a desired therapeutic effect.

In embodiments, the subject does not develop a substantial immune response against the rAAV particle.

In embodiments, the subject does not develop a substantial humoral immune response against the rAAV particle.

In embodiments, the subject does not develop a substantial immune response against the rAAV particle for at least 1, 2, 3, 4, 5, 6, 7, 8, 9, 10, 11, 12, 13, or 14 continuous days, weeks, or months.

In embodiments, the subject does not develop a detectable immune response against the rAAV particle.

In embodiments, the subject does not produce an amount of undesirable immune response against the therapeutic protein and/or the rAAV particle that blocks the therapeutic effect of the therapeutic protein in the subject for at least 1, 2, 3, 4, 5, 6, 7, 8, 9, 10, 11, 12, 13 or 14 continuous days, weeks, or months.

More information about AAV vectors, production of vectors, pharmaceutical compositions including AAV vectors, and methods thereof can be found in U.S. Pat. No. 9,433,688, which is incorporated by reference in its entirety.

In embodiments, the viral vector is an rAAV particle comprising an AAV capsid protein and a vector comprising the nucleic acid encoding an antigen binding molecule inserted between a pair of AAV inverted terminal repeats (ITRs) in a manner effective to infect the cells, organ, or tissue of the peripheral blood system in the subject such that the cells, organ or tissue express antigen binding molecule.

In embodiments, administering the viral vector to a subject comprises infusion or injection of the viral vector into the systemic circulation of the subject. In embodiments, the administering comprises intravenous, intra-arterial infusion, or injection into the systemic circulation of the subject.

In embodiments, the AAV is used to deliver an antigen binding molecule to a subject. The antigen binding molecule is a CAR that comprises an extracellular domain, a transmembrane domain, and an intracellular domain, and the extracellular domain binds an antigen.

In embodiments, the intracellular domain comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.

In embodiments, the intracellular domain comprises CD3 zeta as the signaling region or primary signaling region.

In embodiments, the antigen is Epidermal growth factor receptor (EGFR), Variant Ill of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Rα2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.

In embodiments, the antigen binding molecule is a T Cell Receptor (TCR). In embodiments, the TCR is a modified TCR. In embodiments, the TCR is derived from spontaneously occurring tumor-specific T cells in patients. In embodiments, the TCR binds to a tumor antigen. In embodiments, the tumor antigen comprises CEA, gp100, MART-1, p53, MAGE-A3, or NY-ESO-1. In embodiments, the TCR comprises TCRγ and TCRδ chains or TCRα and TCRβ chains. In embodiments, a T cell clone that expresses a TCR with a high affinity for the target antigen can be isolated. In embodiments, tumor-infiltrating lymphocytes (TILs) or peripheral blood mononuclear cells (PBMCs) can be cultured in the presence of antigen-presenting cells (APCs) pulsed with a peptide representing an epitope known to elicit a dominant T cell response when presented in the context of a defined HLA allele. High-affinity clones can be then selected based on MHC-peptide tetramer staining and/or the ability to recognize and lyse target cells pulsed with low titrated concentrations of cognate peptide antigen. After the clone has been selected, the TCRα and TCRβ chains or TCRγ and TCRδ chains are identified and isolated by molecular cloning. For example, for TCRα and TCRβ chains, the TCRα and TCRβ gene sequences are then used to generate an expression construct that ideally promotes stable, high-level expression of both TCR chains in human T cells. The transduction vehicle, such as a gammaretrovirus or lentivirus, can be then generated and tested for functionality (antigen specificity and functional avidity) and used to produce a clinical lot of the vector. An aliquot of the final product can be used to transduce the target T cell population (generally purified from patient PBMCs), which is expanded before infusion into the patient.

In embodiments, the binding element of the CAR can include any antigen binding moiety that, when bound to its cognate antigen, affects a tumor cell such that the tumor cell fails to grow or is promoted to die or diminish.

The nucleic acid sequences encoding the desired molecules, such as CAR molecules, can be obtained using recombinant methods known in the art, such as, for example, by screening libraries from cells expressing the gene, by deriving the gene from a vector known to include the same, or by isolating directly from cells and tissues containing the same, using standard techniques. Alternatively, the gene of interest can be produced synthetically rather than cloned.

The expression of natural or synthetic nucleic acids encoding CARs is typically achieved by operably linking a nucleic acid encoding the CAR polypeptide or portions thereof to one or more promoters and incorporating the construct into an expression vector. The vectors can be suitable for the replication and integration of eukaryotes. Typical cloning vectors contain transcription and translation terminators, initiation sequences, and promoters useful for regulation of the expression of the desired nucleic acid sequence.

Additional information related to the expression of synthetic nucleic acids encoding CARs and gene transfer into mammalian cells is provided in U.S. Pat. No. 8,906,682, incorporated by reference in its entirety.

The embodiments of the present disclosure further relate to vectors in which a DNA encoding a desired molecule of the present disclosure can be inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Pharmaceutical compositions of the present disclosure can be administered in a manner appropriate to the disease to be treated (or prevented). The quantity and frequency of administration will be determined by such factors as the condition of the patient, and the type and severity of the patient's disease, although appropriate dosages can be determined by clinical trials. As an example, pharmaceutical compositions disclosed herein include nucleic acids encoding CAR or vectors described herein and a pharmaceutically acceptable carrier.

The term “pharmaceutically acceptable” means approved by a regulatory agency of the U.S. Federal or a state government or the EMA (European Medicines Agency) or listed in the U.S. Pharmacopeia (United States Pharmacopeia-33/National Formulary-28 Reissue, published by the United States Pharmacopeial Convention, Inc., Rockville Md., publication date: April 2010) or other generally recognized pharmacopeia for use in animals, and more particularly in humans.

The term “carrier” refers to a diluent, adjuvant (e.g., Freund's adjuvant (complete and incomplete)), excipient, or vehicle with which the therapeutic is administered. Pharmaceutical carriers can be sterile liquids, such as water and oils, including those of petroleum, animal, vegetable, or synthetic origins, such as peanut oil, soybean oil, mineral oil, sesame oil, and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. For the use of (further) excipients and their use see also “Handbook of Pharmaceutical Excipients”, fifth edition, R. C. Rowe, P. J. Seskey and S. C. Owen, Pharmaceutical Press, London, Chicago.

The administration of the pharmaceutical compositions described herein can be carried out in any convenient manner, including by aerosol inhalation, injection, ingestion, transfusion, implantation, or transplantation. The compositions described herein can be administered to a patient subcutaneously, intradermally, intratumorally, intranodally, intramedullary, intramuscularly, by intravenous (i. v.) injection, or intraperitoneally. In embodiments, the T cell compositions of the present disclosure are administered to a patient by intradermal or subcutaneous injection. In another embodiment, the T cell compositions of the present disclosure are preferably administered by i.v. injection. The compositions of T cells can be injected directly into a tumor, lymph node, or site of infection. In embodiments of the present disclosure, cells activated and expanded using the methods described herein, or other methods known in the art where T cells are expanded to therapeutic levels, are administered to a patient in conjunction with (e.g., before, simultaneously, or following) any number of relevant treatment modalities, including but not limited to treatment with agents such as antiviral therapy, cidofovir, and interleukin-2, Cytarabine (also known as ARA-C) or natalizumab treatment for MS patients or efalizumab treatment for psoriasis patients or other treatments for PML patients. In embodiments, the T cells of the present disclosure can be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludarabine, cyclosporin, FK506, rapamycin, mycophenolic acid, steroids, FR901228, cytokines, and irradiation. These drugs inhibit either the calcium-dependent phosphatase calcineurin (cyclosporine and FK506) or inhibit the p70S6 kinase that is important for growth factor-induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In embodiments, the cell compositions of the present disclosure are administered to a patient in conjunction with (e.g., before, simultaneously, or following) bone marrow transplantation, T cell ablative therapy using either chemotherapy agents such as fludarabine, external-beam radiation therapy (XRT), cyclophosphamide, or antibodies such as OKT3 or CAMPATH. In embodiments, the cell compositions of the present disclosure are administered following B-cell ablative therapy, such as agents that react with CD20, e.g., Rituxan. For example, subjects can undergo standard treatment with high dose chemotherapy followed by peripheral blood stem cell transplantation. In embodiments, following the transplant, subjects receive an infusion of the expanded immune cells of the present disclosure. In other embodiments, expanded cells are administered before or following surgery.

The dosage of the above treatments to be administered to a patient will vary with the precise nature of the condition being treated and the recipient of the treatment. The scaling of dosages for human administration can be performed according to art-accepted practices by a physician, depending on various factors.

When “an immunologically effective amount,” “an anti-tumor effective amount,” “a tumor-inhibiting effective amount,” “therapeutic amount,” or “effective amount” is indicated, the precise amount of the compositions of the present disclosure to be administered can be determined by a physician with consideration of individual differences in age, weight, tumor size, the extent of infection or metastasis, and condition of the patient (subject). It can be stated that a pharmaceutical composition comprising the T cells described herein can be administered at a dosage of 10⁴ to 10⁹ cells/kg body weight, preferably 10⁵ to 10⁶ cells/kg body weight, including all integer values within those ranges. T cell compositions can also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art by monitoring the patient for signs of disease and adjusting the treatment accordingly. In embodiments, it can be desired to administer activated T cells to a subject and then subsequently redraw the blood (or have apheresis performed), collect the activated and expanded T cells, and reinfuse the patient with these activated and expanded T cells. This process can be carried out multiple times every few weeks. In embodiments, T cells can be activated from blood draws of from 10 cc to 400 cc. In embodiments, T cells are activated from blood draws of 20 cc, 30 cc, 40 cc, 50 cc, 60 cc, 70 cc, 80 cc, 90 cc, or 100 cc. Not to be bound by theory, certain populations of T cells can be selected using this multiple blood draw/multiple reinfusion protocols.

Additional information on the methods of cancer treatment using engineered or modified T cells is provided in U.S. Pat. No. 8,906,682, incorporated by reference in its entirety.

Embodiments relate to an in vitro method for preparing modified cells. The method can include obtaining a sample of cells from the subject. For example, the sample can include T cells or T cell progenitors. The method can further include transfecting or transducing the cells with a DNA encoding at least a CAR, culturing the population of CAR cells ex vivo in a medium that selectively enhances proliferation of CAR-expressing T cells.

In embodiments, the sample is a cryopreserved sample. In embodiments, the sample of cells is from umbilical cord blood or a peripheral blood sample from the subject. In embodiments, the sample of cells is obtained by apheresis or venipuncture. In embodiments, the sample of cells is a subpopulation of T cells.

The present disclosure describes a method of providing modified cells. The method can comprise obtaining whole blood cells from a subject or a healthy donor, mixing a nucleic acid encoding a CAR or a TCR with the whole blood cells to obtain the modified cells. In embodiments, the modified cells comprise neutrophils. It has been reported that neutrophils promote the production of IL-12 by macrophages, induce UTCαβ into type I activation state, release IFNγ, and exert anti-tumor immune function. In embodiments, the modified cells comprise myeloid cells. In embodiments, the modified cells further comprise a polynucleotide encoding IL-12. Several markers can be used to retain myeloid cells in the blood sample, and examples of the markers include CD33, CD156a, and CD11b. In embodiments, B cells can be removed from the whole blood to reduce the risk of the generation of B cell line cancer. For example, CD19 or CD20 antibodies can be incorporated into system 2400 in FIG. 8.

The present disclosure describes a method of delivering a therapeutic protein to the peripheral blood system of a subject, the method comprising: administering to the peripheral blood system of the subject a viral vector comprising a nucleic acid encoding the therapeutic protein such that cells, organ or tissue of the peripheral blood system express the antigen binding molecule of the subject. In embodiments, the therapeutic protein is an antigen binding molecule. In embodiments, the antigen binding molecule is a CAR.

The present disclosure describes a method of treating cancer in a subject, the method comprising: administering to the peripheral blood system of the subject a viral vector comprising a nucleic acid sequence encoding an antigen binding molecule such that cells, organ, or tissue of the peripheral blood system express the antigen binding molecule of the subject; and monitoring T cell response derived from the expression of antigen binding molecule in the subject.

The present disclosure describes a method of in vivo expression of antigen binding molecule in lymphocytes of a subject, the method comprising: administering into the peripheral blood system of the subject a viral vector, such as the AAV vector, comprising a nucleic acid sequence encoding a CAR, such that cells, organ or tissue of the peripheral blood system express the CAR of the subject.

In embodiments, the nucleic acid sequence encodes a humanized CD19 CAR described in PCT Publication No: WO2018126369, which is incorporated by its entirety.

In embodiments, the nucleic acid sequence encodes a CAR targeting an antigen of a non-essential tissue described in PCT Publication No: WO2018064921, which is incorporated herein by reference in its entirety.

In embodiments, the nucleic acid sequence encodes a CAR and a therapeutic agent described in PCT Publication No: WO2020106843, which is incorporated herein by reference in its entirety.

In embodiments, the nucleic acid sequence encodes one or more components of CoupledCAR® described in PCT Publication No: WO2020146743, which is incorporated herein by reference in its entirety.

In embodiments, the nucleic acid sequence encodes a modified immune checkpoint molecule (e.g., PD-1) as described in U.S. Pat. No. 9,572,837, which is incorporated herein by reference in its entirety. An immune checkpoint molecule refers to a molecule that is associated with T cells and regulates T cell response. In embodiments, the immune checkpoint molecule is selected from the group consisting of PD-1, cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), and CD 160.

The present disclosure describes methods of producing mixed CAR cells from cells of a subject having cancer or a healthy donor. For example, the mixed CAR cells include conventional T cells (T cells expressing TCR), γδT lymphocytes (γδT cells), NKT, and/or NK cells. General information of CAR-engineered cells of each of these cells may be found in Rotolo, R. et al., CAR-Based Strategies beyond T Lymphocytes: Integrative Opportunities for Cancer Adoptive Immunotherapy. Int. J. Mol. Sci. 2019, 20, 2839, which is incorporated by reference in its entirety. CAR NKT cells can effectively localize at the tumor site and presente strong antitumor activity without inducing GvHD. The adoptive transfer of expanded, activated autologous NK cells reported a limited clinical efficacy related to the inhibition by self-HLA molecules, while NK cells from an allogeneic source can hold the potential to be developed as a valid alternative approach. To minimize the occurrence of GvHD, allogeneic NK cells can be obtained from HLA-matched or haploidentical donors.

In embodiments, the method can include obtaining mixed cells including T cells and NK cells, and introducing a polynucleotide encoding a CAR into the mixed cells to obtain mixed CAR cells. For example, anti-CD56 magnetic beads can be implemented to select NK cells. In embodiments, the method can include obtaining T cells and NK cells, separately, introducing the polynucleotide into the T cells and NK cells to obtain CAR T cells and CAR NK cells, and mixing the CAR T cells and CAR NK cells to obtain the mixed CAR cells.

In embodiments, the method can include collecting peripheral blood mononuclear cells (PBMCs) from the subject and selecting CD3+ cells from the PBMCs. In embodiments, the PBMCs can be mixed with a group of antibodies to allow the group of antibodies to bind target cells. In embodiments, the group of antibodies does not include CD3 antibodies. The target cells can then be removed from the PBMCs to obtain a composition of cells containing the CD3+ cells. For example, the group of antibodies can include at least one of CD14, CD15, CD16, CD19, CD34, CD36, CD56, CD123, or CD235a. The CD3+ cells can then be cultured with anti-CD3 and anti-CD28 beads and introduced with a polynucleotide encoding a CAR to obtain CAR T cells. The method can further comprise obtaining CAR NK cells from the healthy donor. For example, the method can include collecting PBMCs from the healthy donor and selecting for CD56+ cells from the PBMCs, which are then introduced with a polynucleotide encoding the CAR to obtain the CAR NK cells. The CAR T cells from a subject having cancer and CAR NK cells from a healthy donor can be mixed based on a predetermined ratio to obtain the mixed CAR cells, which are then infused into the subject. For example, the ratios between the CAR T cells and CAR NK cells can comprise 1:10 to 1:1000,000 (e.g., 1:100, 1:1,000, 1:10,00, and 1:10,000).

In embodiments, the method can include collecting peripheral blood mononuclear cells (PBMCs) from the subject and selecting CD3+ cells and CD56+ from the PBMCs, which are then introduced with a polynucleotide encoding the CAR to obtain both CAR T cells and CAR NK cells. These mixed CAR cells are then infused into the subject. In embodiments, whole blood cells or PMBCs can be introduced with a polynucleotide encoding CAR, and CAR T cells and/or CAR NK cells can be selected for infusion back into the patient. In embodiments, IL-15 can be added to the mixed cells for helping in vitro culturing of NK cells.

In embodiments, substantially whole blood can be used in the above embodiments. Substantially whole blood is the blood that is isolated from an individual(s), has not been subjected to a PBMC enrichment procedure, and is diluted by less than 50% with other solutions. For example, this dilution can be from the addition of an anti-coagulant as well as the addition of a volume of fluid comprising retroviral particles.

TABLE 2
Examples of the Mixtures and their components

| | Mixture | Mixture | Mixture | Mixture |
|---|---|---|---|---|
| | component (I) | component (II) | component (III) | component (VI) |
| Activation | CD3/CD28 | CD3/CD28 | CD3/CD28 | CD3/CD28 |
| agents | beads | beads | beads | beads |
| Vectors | Vector encoding | Vector encoding | Vector encoding | Vector encoding |
| | CD19 CAR, | CD19 CAR, | CD19 CAR, | CD19 CAR, |
| | NFAT-IL6 | NFAT-IL6-2A- | NFAT-IL6 | NFAT-IL6 |
| | Vector encoding | IFNγ | Vector encoding | Vector encoding |
| | CD19 CAR, | Vector encoding | solid tumor, | CD19 CAR, |
| | NFAT-IL12 | CD19 CAR, | NFAT-IL12 | NFAT-IL12 |
| | Vector encoding | NFAT-IL12 | Vector encoding | Vector encoding |
| | CD19 CAR, | Vector encoding | solid tumor, | CD19 CAR, |
| | NFAT-IFNγ | solid tumor CAR | NFAT-IFNγ | NFAT-IFNγ |
| | Vector encoding | | Vector encoding | Vector encoding |
| | solid tumor CAR | | solid tumor CAR | solid tumor, |
| | | | | NFAT-IL12 |
| | | | | Vector encoding |
| | | | | solid tumor, |
| | | | | NFAT-IFNγ |
| | | | | Vector encoding |
| | | | | solid tumor CAR |

Embodiments relate to a system for immune therapy or inducing a T cell response, the system comprising: a sample processing module, for example, a blood collection apparatus (FIG. 14) configured to obtain T cells from a blood sample from a subject; a cell incubation module, for example, a blood processing apparatus (FIG. 14) configured to activate the T cells and introduce a vector into the T cells; and a cell infusion module, for example, cell process apparatus (FIG. 14) configured to infuse at least a portion of the T cells into the subject, wherein: the sample processing module comprises: an input port configured to receive the blood sample from the subject; a sample separation module configured to: separate the T cells from remaining components of the blood sample, collecting the T cells; an output port configured to: flow the T cells to the cell incubation module, and wherein the sample separation module is configured to: contact the blood sample with a composition comprising T cell aggregation reagent comprising T cell recognizing moiety coupled to a magnetic particle or antigen recognizing moieties specifically binding undesired cellular components; apply simultaneously gravity sedimentation for sedimentation of T cells or undesired cells and a magnetic field gradient to the blood sample for immobilizing the magnetic particle; generate a pellet and a supernatant phase; and recover the desired cells from the supernatant phase or the pellet.

In embodiments, inducing T cell response includes stimulating or activating a T cell response.

Embodiments relate to a system for immune therapy or causing a T cell response, the system comprising: a sample processing module configured to obtain blood cells from a blood sample from a subject; a cell incubation module configured to activate blood cells and/or introduce a vector into the blood cells; and a cell infusion module configured to infuse at least a portion of the transduced blood cells to the subject, wherein the blood cells comprising CD3+ cells.

In embodiments, the sample processing module is coupled to the cell incubation module that is coupled to the cell infusion module such that the blood cells flow through the system starting from the subject and ending back to the subject.

In embodiments, the sample or blood processing module, an example of which is shown in FIG. 14, comprises: an input port configured to receive the blood sample from the subject; a sample separation module configured to separate the blood cells from remaining components of the blood sample, and collect the blood cells; an output port configured to flow the blood cells to the cell incubation module, and flow the remaining components of the blood sample into the subject.

In embodiments, the sample separation module is configured to: contact the blood sample with a composition comprising a CD3+ aggregation reagent comprising CD3 recognizing moiety coupled to a magnetic bead and antigen recognizing moieties specifically binding undesired cellular components; apply gravity sedimentation for sedimentation of CD3+ cells or undesired cells and a magnetic field gradient to the blood sample for immobilizing the magnetic bead simultaneously; generate a pellet and a supernatant phase; and recover the desired CD3+ cells from the supernatant phase or the pellet.

In embodiments, the system comprises components that are composed of plastic. Examples of such components include containers, storage bags, tubes and such as inlet and outlet tubes. The plastic can comprise polystyrene, polyvinylchloride, polycarbonate, glass, polyacrylate, polyacrylamide, polymethylmethacrylate (PMMA), polyethylene terephthalate (PET), polytetrafluoroethylene (PTFE), thermoplastic polyurethane (TPU), silicone, polyethylene (PE), polylactide (PLA), polyglycolide (PGA), and their copolymers. In embodiments, the components of the system comprise collagen, chitin, alginate, hyaluronic acid, and hyaluronic acid derivatives, In embodiments, the system includes components that comprise ceramics, glass materials, hydroxyapatite (HA), and calcium phosphate. In embodiments, the system comprises compositions comprising one or more of the above-mentioned materials.

In embodiments, the system further comprises one or more sensors configured to detect the progress of separation of the blood sample from the subject, in particular by detecting the formation of layers, such as the gradient, of the blood sample, the change of pH value of the blood sample, and/or the change in temperature of the blood sample.

In embodiments, the cell processing or incubation module (FIG. 14) comprises a rotating container or cell processing apparatus configured to culture cells and/or grow cells. The rotating container is disposable and/or has been sterilized.

Embodiments relate to a novel method of immune therapy or inducing a T cell response by implementing a system described herein, the method comprising: receiving peripheral blood of a subject; obtaining the CD3+ cells from the peripheral blood; infusing the remaining blood components of the peripheral blood back into the subject; obtaining vectors comprising a nucleic acid encoding a CAR and one or more agents that activate T cells; mixing the vectors and the obtained CD3+ cells and incubating the mixture for a predetermined time to introduce the nucleic acid into the CD3+ cells; replacing the culturing media of the CD3+ cells with a solution suitable for infusion into the subject, and infusing at least a portion of the CD3+ cells into the subject. In embodiments, the method is completed less than 1, 2, 3, 4, or 5 days. In embodiments, the method is completed in less than 72 hours (hrs), 24 hrs, 20 hrs, 16 hrs, 12 hrs, 10 hrs, 8 hrs, or 6 hrs. In embodiments, the method is completed in 30 minutes (mins) to 24 hrs, 30 mins to 5 hrs, 30 mins to 3 hrs, 1 hr to 24 hrs, 2 to 4 hrs, 2 to hrs, 5 to 24 hrs, 5 to 20 hrs, 5 to 16 hrs, 5 to 10 hrs, or 5 to 8 hrs.

Embodiments relate to an apparatus for sample processing or inducing a T cell response, the apparatus comprising: an input port configured to receive blood sample from a subject; a sample separation module configured to isolate desired cells from the blood sample; and an output port configured to: flow the desired cells to the cell incubation module for further processing, and flow undesired cells and/or remaining components of the blood sample back into the subject. In embodiments, the desired cells are blood cells, such as CD3+ cells, and the blood sample is peripheral blood.

In embodiments, the sample processing module can further comprise a unit configured to obtain cells from the peripheral blood of the subject, for example a human patient. In embodiments, the unit is an apheresis device in which the blood of a subject is passed through an apparatus that separates out one particular constituent and returns the remainder to the circulatory system of the subject.

Embodiments relate to a method for sample processing or inducing a T cell response by implementing any of the apparatus, systems, or devices described herein, the method comprising: receiving a blood sample from a subject; isolating desired cells from the blood sample; flowing the desired cells for further processing; flowing undesired cells and/or remaining components of the blood sample back into the subject; introducing a nucleic acid sequence encoding an antigen binding molecule into the desired cell; and administering an effective amount of a composition comprising the desired cells including the antigen binding molecule into the subject. In embodiments, the desired cells administered to the subject comprise CD3+ cells.

In conventional CAR T therapy, the vein-to-vein time, which is the collection of T cells to CAR T cell infusion, can take a few weeks, for example three to five weeks. In embodiments, with the novel CAR T therapy described herein, a vein-to-vein time is between 30 minutes and 1 hr, 1 hr and 72 hrs, 1 hr and 12 hrs, 1 hr and 24 hrs, 1 hr and 48 hrs, 12 hrs and 24 hrs, 12 hrs and 48 hrs, or 48 hrs and 72 hrs (including numbers between). In embodiments, the vein-to-vein time is between 30 minutes (mins) to 24 hrs, 30 mins to 5 hrs, 30 mins to 3 hrs, 1 hr to 24 hrs, 2 to 4 hrs, 2 to hrs, 5 to 24 hrs, 5 to 20 hrs, 5 to 16 hrs, 5 to 10 hrs, or 5 to 8 hrs.

In embodiments, the vein-to-vein time is within 12 hrs or 24 hrs.

In embodiments, activation of the collected cells and introduction of vectors into cells are performed at the same time.

In embodiments, the vectors introduced into the cells comprise a polynucleotide encoding a CAR targeting a WBC antigen, for example, CD19, CD20, CD22, and BCMA, a second polynucleotide encoding a CAR targeting a solid tumor antigen, such as one listed in Table 1, a third polynucleotide encoding IL-12, a fourth polynucleotide encoding IL-6, and/or a polynucleotide encoding IFN-γ.

In embodiments, the vectors introduced into the cells comprise a polynucleotide encoding a CAR targeting a WBC antigen, such as CD19, CD20, CD22, and BCMA, a second polynucleotide encoding a CAR targeting a solid tumor antigen, such as one listed in Table 1, and a third polynucleotide encoding a cytokine. In embodiments, the vectors introduced into the human cells comprise a first vector comprising a polynucleotide encoding a CAR targeting a WBC antigen, for example, CD19, CD20, CD22, and BCMA, and a second vector comprising a polynucleotide encoding a CAR targeting a solid tumor antigen, for example, an antigen listed in Table 1, wherein the first and second polynucleotides are on separate vectors.

In embodiments, the cells infused into the subject comprise transduced T cells comprising a CAR targeting a WBC antigen and transduced T cells comprising a CAR targeting a solid tumor. In embodiments, the transduced T cells comprise a CAR targeting a WBC antigen that does not comprise a CAR targeting the solid tumor, and the transduced T cells comprise a CAR targeting a solid tumor that does not comprise the CAR targeting the WBC antigen.

In embodiments, the cells infused into the subject comprise one or more vectors listed in Table 2.

In embodiments, the blood cells for sample processing are obtained from a human patient as described herein and processed as described herein for infusion back into a human patient. In embodiments, the human patient which the blood cells are obtained from is the same human patient that is receiving the infusion of the processed cells. In embodiments, the cells are collected from a subject and infused into a subject are human cells.

In embodiments, the CAR comprises an extracellular domain, a transmembrane domain, and an intracellular domain, and the extracellular domain binds an antigen. In embodiments, the intracellular domain comprises a signaling domain or primary signaling domain such as CD3 zeta. In embodiments, the intracellular domain comprises a co-stimulatory signaling region that comprises an intracellular domain of a co-stimulatory molecule selected from the group consisting of CD27, CD28, 4-11BB, OX40, CD30, CD40, PD-1, ICOS, lymphocyte function-associated antigen-1 (LFA-1), CD2, CD7, LIGHT, NKG2C, B7-H3, and any combination thereof. In embodiments, the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Rα2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.

In embodiments, the vectors described herein comprise a nucleic acid sequence encoding a binding molecule. In embodiments, the binding molecule is a CAR or TCR. In embodiments, the vector is a lentiviral vector.

Embodiments relate to a method of delivering a therapeutic protein to the peripheral blood system of a subject, the method comprising: administering to the peripheral blood system of the subject a viral vector comprising a nucleic acid sequence encoding an antigen binding molecule, such that cells, organ or tissue of the peripheral blood system express the antigen binding molecule in the subject.

In embodiments, the nucleic acid encoding the therapeutic protein described herein has reduced cytosine-guanine dinucleotide (CpG).

In embodiments, the nucleic acid encoding the therapeutic protein described herein has reduced cytosine-guanine dinucleotide (CpG) as compared to a wild-type nucleic acid encoding the therapeutic protein.

Embodiments relate to a method of treating cancer in a subject, the method comprising: administering to the peripheral blood system of the subject a viral vector comprising a nucleic acid sequence encoding a chimeric antigen receptor such that cells, organ or tissue of the peripheral blood system express the antigen binding molecule of the subject; and monitoring T cell response resulting from the expression of CAR in the subject.

Embodiments relate to a method of in vivo expression of antigen binding molecule in lymphocytes of a subject, the method comprising: administering to the peripheral blood system of the subject a viral vector comprising a nucleic acid sequence encoding a CAR such that cells, organ or tissue of the peripheral blood system express the CAR in the subject.

In embodiments, the viral vector is an rAAV particle comprising an AAV capsid protein and a vector comprising the nucleic acid encoding antigen binding molecule inserted between a pair of AAV inverted terminal repeats (ITRs) in a manner effective to infect the cells, organ, or tissue of the peripheral system in the subject such that the cells, organ or tissue express the antigen binding molecule.

In embodiments, the vector further comprises one or more of an intron, an expression control element, a filler polynucleotide sequence and/or poly A signal, or a combination thereof.

In embodiments, the pair of ITRs are derived from or comprises an ITR sequence of any of AAV1, AAV2, AAV3, AAV4, AAV5, AAV6, AAV7, AAV8, AAV9, AAV10, AAV11, AAV 12, LK01, LK02, LK03, AAV 4-1, AAV-2i8 ITRs, or a mixture thereof.

In embodiments, the AAV capsid protein is derived from or comprise a capsid protein sequence any of: AAV1, AAV2, AAV3, AAV4, AAV5, AAV6, AAV7, AAV8, AAV9, AAV10, AAV11, AAV 12, LK01, LK02, LK03, AAV 4-1, AAV-2i8 ITRs, or a mixture thereof.

In embodiments, the rAAV particles are administered at a dose of about 5×10¹¹ vector genomes per kilogram (vg/kg) of the subject.

In embodiments, the subject does not develop a substantial immune response against the rAAV particle. In embodiments, the subject does not develop a substantial humoral immune response against the rAAV particle. In embodiments, the subject does not develop a substantial immune response against the rAAV particle for at least 1, 2, 3, 4, 5, 6, 7, 8, 9, 10, 11, 12, 13, or 14 continuous days, weeks, or months.

In embodiments, the subject does not develop a detectable immune response against the rAAV particle. In embodiments, the subject does not produce an immune response against the therapeutic protein and/or the rAAV particle sufficient to block the therapeutic effect in the subject for at least 1, 2, 3, 4, 5, 6, 7, 8, 9, 10, 11, 12, 13 or 14 continuous days, weeks or months.

In embodiments, administering the viral vector comprises infusion or injection of the viral vector into the systemic circulation of the subject. In embodiments, the administering the viral vector comprises intravenous or intra-arterial infusion or injection of the viral vector into the systemic circulation of the subject.

In embodiments, the viral vector further comprises a nucleic acid encoding IL-12, such that anti-tumor activities resulting from the administration of the viral vector are enhanced as compared to a subject that is administered with the viral vector without the nucleic acid encoding IL-12, and wherein the expression of IL-12 is regulated by NFAT response element.

In embodiments, the method further comprises administering to the subject an effective amount of CD28 and CD3 agonists and/or protamine sulfate, wherein transduction ratios and anti-tumor activities are enhanced as compared with a subject that is administered with the viral vector without the arsonists and protamine sulfate. In embodiments, agonists of CD28 and CD3 includes their respective antibodies.

Embodiments relate to a modified cell generated or processed by any suitable method, apparatus, device, or systems described herein.

In embodiments, the modified cell comprises an antigen binding molecule, wherein expression and/or function of one or more molecules in the modified cell has been enhanced or reduced including eliminated, and wherein the one or more molecules comprising at least one of G-CSF, GM-CSF, and a derivative of G-CSF or GM-CSF.

Embodiments relate to a method or use of polynucleotide, the method comprising: providing a viral particle, for example, AAV, lentivirus or their variants, comprising a vector genome, the vector genome comprising the polynucleotide encoding G-CSF, GM-CSF, and a derivative of G-CSF or GM-CSF and a polynucleotide encoding an antigen binding molecule, the polynucleotide operably linked to an expression control element regulating the transcription of the polynucleotide; and administering an amount of the viral particle to a subject such that the polynucleotide is expressed in the subject, wherein the one or more molecules are overexpressed in cancer cells, associated with recruitment of immune cells, and/or associated with autoimmunity.

Embodiments relate to a method of eliciting or enhancing T cell response, treating a subject in need thereof or enhancing cancer treatment thereof, the method comprising administering an effective amount of the composition to the subject.

In embodiments, the nucleic acid is an mRNA, which is not integrated into the genome of the modified cell. In embodiments, the nucleic acid is associated with an oxygen-sensitive polypeptide domain. In embodiments, the oxygen-sensitive polypeptide domain comprises HIF VHL binding domain.

In embodiments, the nucleic acid sequence is regulated by a promoter comprising a binding site for a transcription modulator that modulates the expression and/or secretion of the therapeutic agent in the cell. In embodiments, the transcription modulator is or includes Hif1a, NFAT, FOXP3, and/or NFkB.

In embodiments, the one or more molecules comprise at least one of G-CSF or GM-CSF, or a combination thereof. In embodiments, one or more molecules comprise at least one of a receptor of G-CSF or GM-CSF, or a combination thereof. In embodiments, the one or more molecules comprise at least one of IL-33, IL-1β, TNFα, MALP-2, IL1, and IL17.

In embodiments, the modified cell comprises the antigen binding molecule, wherein the antigen binding molecule is a chimeric antigen receptor (CAR) comprising an antigen binding domain, a transmembrane domain, and an intracellular signaling domain.

In embodiments, the antigen binding domain binds to a tumor antigen selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase (hTERT), RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In embodiments, the intracellular domain comprises a signaling domain. In embodiments, the intracellular signaling domain comprises a signaling domain, or a primary signaling domain and a co-stimulatory signaling domain, wherein the signaling domain, the primary signaling domain or the co-stimulatory signaling domain comprises a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In embodiments the signaling domain or the primary signaling domain comprises CD3 zeta.

In embodiments, the modified cell comprises the antigen binding molecule, the antigen binding molecule is a modified TCR. In embodiments, the TCR is derived from spontaneously occurring tumor-specific T cells in patients. In embodiments, the TCR binds to a tumor antigen. In embodiments, the tumor antigen comprises CEA, gp100, MART-1, p53, MAGE-A3, or NY-ESO-1. In embodiments, the TCR comprises TCRγ and TCRδ Chains or TCRα and TCRβ chains, or a combination thereof.

In embodiments, the modified cell is derived from an immune cell (e.g., a population of immune effector cells). In embodiments, the immune cell is a T cell or an NK cell. In embodiments, the immune effector cell is a T cell. In embodiments, the T cell is a CD4+ T cell, a CD8+ T cell, or a combination thereof. In embodiments, the cell is a human cell.

In embodiments, the modified cell comprises a nucleic acid sequence encoding a binding molecule and a dominant negative form of an inhibitory immune checkpoint molecule or a receptor thereof. In embodiments, the inhibitory immune checkpoint molecule is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), and CD 160.

In embodiments, the inhibitory immune checkpoint molecule is modified PD-1. In embodiments, the modified PD-1 lacks a functional PD-1 intracellular domain for PD-1 signal transduction; interferes with a pathway between PD-1 of a human T cell of the human cells and PD-L1 of a certain cell; comprises or is a PD-1 extracellular domain, a PD-1 transmembrane domain, or a combination thereof; comprises or is a modified PD-1 intracellular domain comprising a substitution or deletion as compared to a wild-type PD-1 intracellular domain; or comprises or is a soluble receptor comprising a PD-1 extracellular domain that binds to PD-L1 of a certain cell.

In embodiments, the modified cell is engineered to express and secrete a therapeutic agent such as a cytokine. In embodiments, the cytokine is or comprises IL-6 or IFN-γ, or a combination thereof. In embodiments, the cytokine is or comprises IL-15 or IL-12, or a combination thereof.

In embodiments, the modified cell is engineered to express or secrete a small protein such as a recombinant or native cytokine. In embodiments, the small protein is or comprises IL-12, IL-6, or IFN-γ.

In embodiments, the modified cell is derived from a healthy donor or the subject having cancer.

In embodiments, the modified cell has a reduced expression of the endogenous T cell receptor alpha constant (TRAC) gene.

In embodiments, the modified cell comprises a first CAR binding an antigen of a white blood cell and a second CAR binding a solid tumor antigen. In embodiments, the modified cell comprises a bispecific CAR binding a white blood antigen and a solid tumor antigen.

Embodiments relate to a pharmaceutical composition comprising a population of the modified cells and a population of additional modified cells, wherein the population of modified cells binds a first antigen, and the population of additional modified cells binds a second antigen, which is different from the first antigen. In embodiments, the first antigen is a WBC antigen, and the second antigen is a solid tumor antigen. In embodiments, the second antigen is a WBC antigen, and the first antigen is a solid tumor antigen.

In embodiments, the WBC antigen is or comprises CD19, CD22, CD20, BCMA, CD5, CD7, CD2, CD16, CD56, CD30, CD14, CD68, CD11b, CD18, CD169, CD1c, CD33, CD38, CD138, or CD13. In embodiments, the WBC antigen is or comprises CD19, CD20, CD22, or BCMA.

In embodiments, the solid tumor antigen comprises tMUC 1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, CLDN18.2, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, B7-H3, or EGFR. In embodiments, the solid tumor antigen comprises tumor-associated MUC1, ACPP, TSHR, GUCY2C, UPK2, CLDN18.2, PSMA, DPEP3, CXCR5, B7-H3, MUC16, SIGLEC-15, CLDN6, Muc17, PRLR, or FZD10.

Embodiments relate to a method of inducing or enhancing T cell response, treating a subject in need thereof or enhancing cancer treatment thereof, the method comprising administering an effective amount of the pharmaceutical composition comprising modified T cells. In embodiments, the modified T cells comprise a nucleic acid encoding hTERT, SV40LT, or a combination thereof. In embodiments, the modified T cells are more proliferable than T cells without the nucleic acid encoding hTERT, SV40LT, or a combination thereof. In embodiments, the proliferable T cells retain functions of normal T cells/CAR T cells, for example, cell therapy functions.

In embodiments, integration of the nucleic acid encoding hTERT, the nucleic acid encoding SV40LT, or a combination thereof includes genomic integration of the nucleic acid encoding hTERT, a nucleic acid encoding SV40LT, or a combination thereof and constitutive expression of hTERT, SV40LT, or a combination thereof. In embodiments, expression of hTERT, SV40LT, or a combination thereof, is regulated by an inducible expression system such as a rtTA-TRE system.

In embodiments, the modified T cells comprise a CAR and the nucleic acid encoding hTERT, nucleic acid encoding SV0LT, or a combination thereof, and the modified T cells are cultured in the presence of an agent that is recognized by the extracellular domain of the CAR.

In embodiments, the modified T cells comprise a nucleic acid sequence encoding a suicide gene such as an HSV-TK system.

In embodiments, the modified T cells have reduced graft-versus-host disease (GVHD) response in a bioincompatible human recipient as compared to the GVHD response of the primary human T cell.

In embodiments, the modified T cells have reduced expression of the endogenous TRAC gene.

In embodiments, the blood sample from the subject includes peripheral blood, which is activated by a T cell activator (e.g., beads conjugated with anti-CD3 and beads conjugated with anti-CD28) and/or introduced with a polynucleotide encoding a binding molecule, such as a CAR or TCR.

In embodiments, CD28 antibodies can be replaced by CD80 or its extracellular domain.

Embodiments relate to a method of enhancing in vivo transduction of T cells or enhancing a gene therapy, the method comprising: administering to a subject having a form of cancer an effective amount of a pharmaceutical composition comprising a viral vector comprising a nucleic acid encoding a CAR or TCR and one or more compounds, the one or more compounds comprising a T cell activator and/or a transduction adjuvant; and allowing the viral vector to be introduced into the T cells of the subject, wherein transduction rate of the nucleic acid into the T cells is higher than the transduction rate of nucleic acid into T cells of a subject that is administered with an effective amount of a pharmaceutical composition comprising the viral vector without the one or more compounds.

Embodiments relate to a method of performing or enhancing a gene therapy, the method comprising: administering to a subject having a form of cancer with an effective amount of a pharmaceutical composition comprising a viral vector comprising a nucleic acid encoding a CAR or TCR and one or more compounds, the one or more compounds comprising a T cell activator and a transduction adjuvant; and allowing the viral vector to be introduced into the T cells of the subject.

In embodiments, the methods related to gene therapies herein can be combined with techniques associated with CoupledCAR® described in PCT Publication Nos: WO2020106843 and WO2020146743, which are incorporated by their entirety. In embodiments, the viral vector(s) can include a nucleic acid encoding a CAR targeting a WBC antigen, for example, CD19 and BCMA, and a nucleic acid encoding a CAR targeting a solid tumor antigen, for example, GCC and TSHR. In embodiments, the viral vector(s) can include (1) a nucleic acid encoding a CAR targeting a WBC antigen, such as CD19 and BCMA, and IL-6, a nucleic acid encoding a CAR targeting the WBC antigen and IL-12, or a nucleic acid encoding a CAR targeting aWBC antigen and IFN-γ, or a combination thereof, and (2) a nucleic acid encoding a CAR targeting a solid tumor antigen, for example, GCC and TSHR.

In embodiments, the intracellular domain of the CAR can comprise a primary signaling domain, a co-stimulatory domain, and a domain associated with the signaling of IL-2R or an exogenous JAK-binding motif, such as the JAK-STAT domain. In embodiments, the term “exogenous association motif” refers to any association motif that is recombinantly introduced into a domain, for example, an intracellular signaling domain such as a cytoplasmic domain of an interleukin receptor chain, a cytoplasmic of a co-stimulatory domain, or a CD34 intracellular signaling domain, but that does not naturally exist in the molecule or at the specific location in the molecule. For example, an exogenous JAK-binding motif can be inserted into an intracellular signaling domain, such as a cytoplasmic domain of an interleukin receptor chain. The “JAK-binding motif” used herein refers to a BOX-1 motif that allows for tyrosine kinase JAK association, for example, JAK1. The JAK-binding motif can be, for example, amino acid numbers 278 to 286 of NCBI RefSeq: NP_000869.1. In this instance, a “domain” means one region in a polypeptide, for example, which is folded into a particular structure independently of other regions and/or has a particular function. The domain can, for example, be the cytoplasmic portion of a molecule or a part thereof. As used herein, the “cytoplasmic domain” of a molecule can refer to the full cytoplasmic domain or a part thereof that induces an intracellular signal when activated.

The term “variant” refers to a molecule comprising a substitution, deletion, or addition of one or a few to a plurality of amino acids and includes particularly conservatively substituted molecules, provided that the variant substantially retains the same function as the original sequence. For example, the variants of the interleukin (IL) receptor can comprise substitutions, deletions, or additions outside the JAK-binding motif and the STAT association motif. Thus, an IL receptor variant can comprise up to 50, up to 40, up to 30, up to 20, or up to 10 amino acid deletion and/or conservative substitutions in a region outside of the JAK-binding and STAT association motifs. Similarly, variants of other molecules can comprise up to 50, up to 40, up to 30, up to 20, or up to 10 amino acid deletion and/or conservative substitutions, in a region outside of a region identified specifically herein. As used herein, the phrase “wherein the intracellular segment comprises an endogenous or exogenous JAK-binding motif and a STAT5 association motif” includes the intracellular segment comprising more than one cytoplasmic domain, and the JAK binding motif and the STAT5 association motif can be in the same cytoplasmic domain or can be in separate cytoplasmic domains. In embodiments, the viral vector(s) can include a nucleic acid encoding IL2R such that white blood cells (e.g., T cells) can overexpress IL2R, enhancing the expansion of the white blood cells.

In embodiments, the transduction adjuvant is or comprises protamine sulfate (PS), optionally from 1 micro g/ml to 50 micro g/ml or from about 1 micro g/ml to about 50 micro g/ml protamine sulfate; a fibronectin-derived transduction adjuvant; and/or RetroNectin.

In embodiments, the T cell activator comprises anti-CD3 and/or anti-CD28 antibodies.

In embodiments, the one or more compounds comprise antibodies or agonists binding CD3 and/or CD28 and PS.

In embodiments, the viral vector comprises a nucleic acid encoding IL-12, wherein anti-tumor activities in the subject are greater than in a subject that is administered with a viral vector comprising the nucleic acid encoding the CAR in the absence of the nucleic acid encoding IL-12.

Embodiments relate to a method for (1) inducing or causing a T cell response in a subject, (2) expanding T cells in a subject, (3) inhibiting the growth of tumor cells in a subject, (4) genetically modifying lymphocytes (e.g., NK or T cells), and/or (5) treating tumors in a subject, the method comprising: administering to the subject an effective amount of a recombinant virus comprising a nucleic acid encoding a chimeric antigen receptor (CAR). In embodiments, the recombinant virus further comprises a nucleic acid encoding a therapeutic agent (e.g., cytokines). In embodiments, the subject has liquid cancer, such as blood cancer (Non-Hodgkin's Lymphoma (NHL)). In embodiments, the CAR binds a WBC antigen (e.g., CD19, CD20, BCMA). In embodiments, the method further comprises administering a therapeutic agent such as IL-12.

Embodiments relate to a method for (1) inducing or causing a T cell response in a subject, (2) expanding T cells in a subject, (3) inhibiting the growth of tumor cells in a subject, (4) genetically modifying lymphocytes, and/or (5) treating tumors in a subject, the method comprising: administering to the subject an effective amount of a recombinant virus carrying a nucleic acid sequence encoding a first CAR binding a first antigen and a second CAR binding a second antigen.

Embodiments relate to a method for (1) inducing or causing a T cell response in a subject, (2) expanding T cells in a subject, (3) inhibiting the growth of tumor cells in a subject, (4) genetically modifying lymphocytes, and/or (5) treating tumors in a subject, the method comprising: administering to the subject an effective amount of a first recombinant virus carrying a nucleic acid sequence encoding a first CAR binding a first antigen and a second recombinant virus carrying a nucleic acid sequence encoding a second CAR binding a second antigen. In embodiments, the first antigen comprises a cell surface molecule of a WBC, a tumor antigen, or a solid tumor antigen.

In embodiments, the WBC is a granulocyte, a monocyte, or a lymphocyte. In embodiments, the WBC is a B cell. In embodiments, the cell surface molecule of the WBC is CD19, CD22, CD20, BCMA, CD5, CD7, CD2, CD16, CD56, CD30, CD14, CD68, CD11b, CD18, CD169, CD1c, CD33, CD38, CD138, or CD13. In embodiments, the cell surface molecule of the WBC is CD19, CD20, CD22, or BCMA. In embodiments, the cell surface molecule of the WBC is CD19 or BCMA.

In embodiments, the tumor antigen comprises a solid tumor antigen. In embodiments, the solid tumor antigen comprises tumor associated MUC1 (tMUC1), PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLECI5, SLC6A3, KISS1R, CLDN18.2, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, B7-H3, or EGFR. In embodiments, the solid tumor antigen comprises tMUC1, ACPP, TSHR, GUCY2C, UPK2, CLDN18.2, PSMA, DPEP3, CXCR5, B7-H3, MUC16, SIGLEC-15, CLDN6, Muc17, PRLR, or FZD10. In embodiments, the solid tumor antigen comprises tMUC1, ACPP, TSHR, GUCY2C, UPK2, or CLDN18.2.

In embodiments, the CAR comprises an antigen binding domain, a transmembrane domain, a co-stimulatory domain, and a CD3 zeta domain. In embodiments, the co-stimulatory domain comprises the intracellular domain of CD27, CD28, 4-1 BB, OX40, CD30, CD40, PD-1, ICOS, lymphocyte function-associated antigen-1 (LFA-1), CD2, CD7, LIGHT, NKG2C, B7-H3, a ligand that binds CD83, or a combination thereof.

In embodiments, the first CAR comprises an scFv binding CD19, an intracellular domain of 4-1 BB or CD28, and CD3 zeta domain, and the second CAR comprises an scFv binding tMUC1, ACPP, TSHR, GUCY2C, or CLDN18.2., an intracellular domain of 4-1 BB or CD28, and CD3 zeta domain.

In embodiments, the nucleic acid sequence further encodes a dominant negative form of PD-1.

In embodiments, the nucleic acid sequence further encodes a therapeutic agent. In embodiments, the therapeutic agent comprises a cytokine. In embodiments, the cytokine is IL6 and/or INF-γ. In embodiments, the cytokine is at least one of IL6, IL12, IL-15, IL-7, TNF-α, or IFN-γ.

In embodiments, the nucleic acid sequence is under the control of a promoter sequence which expresses the product of the nucleic acid sequence in lymphocytes.

In embodiments, the recombinant virus is an adenovirus; an adeno-associated virus (AAV); a retrovirus, for example, MMLV; or a lentivirus, for example, HIV-1, FIV, or SIV. In embodiments, the recombinant virus is a lentivirus. In embodiments, the recombinant virus is an AAV (rAAV). In embodiments, the effective amount comprises 1×10⁹ to 2×10¹² rAAV.

In embodiments, the promoter is a cell-specific promoter. In embodiments, the promoter is the chicken beta-actin promoter/CMV enhancer.

Embodiments relate to a pharmaceutical composition comprising at least one of the recombinant viruses described herein.

Embodiments relate to a pharmaceutical composition for (1) inducing or causing T cell response in a subject, (2) expanding T cells in a subject, (3) inhibiting growth of tumor cells in a subject, (4) genetically modifying lymphocytes, (5) treating tumors in a subject, and/or (6) enhancing T cell response, T cell expansion in vivo, inhibition growth of tumor cells, and/or treatment of the tumor. The pharmaceutical composition comprises an effective amount of one or more viruses or recombinant viruses described herein carrying a nucleic acid sequence encoding a first CAR described herein binding a first antigen and a second CAR described herein binding a second antigen.

Embodiments relate to a method for (1) inducing T cell response in a subject, (2) expanding T cells in a subject, (3) inhibiting the growth of tumor cells in a subject, (4) genetically modifying lymphocytes, and/or (5) treating tumors in a subject. The method comprises administering to the subject an effective amount of the modifying lymphocytes described herein. In embodiments, the method further comprises administering to the subject an effective amount of an agent that activates T cells to allow the introduction of the recombinant virus(es).

In embodiments, the agent that activates T cells comprises a T cell activator, for example, beads conjugated with anti-CD3 and beads conjugated with anti-CD28. In embodiments, the agent comprises Transact™.

In embodiments, the recombinant virus(es) are in the form of a viral particle, such as a replication-incompetent recombinant retroviral particle, that comprises a membrane-bound T cell-activating element on the surface of the particle (more information about the replication-incompetent recombinant retroviral particle can be found in PCT Patent Publication No: WO2018009923 and WO2018161064, which are incorporated herein in its entirety).

In embodiments, the membrane-bound T cell-activating element comprises or is anti-CD3 scFv.

In embodiments, viral particle further comprises a membrane-bound polypeptide capable of binding to CD28.

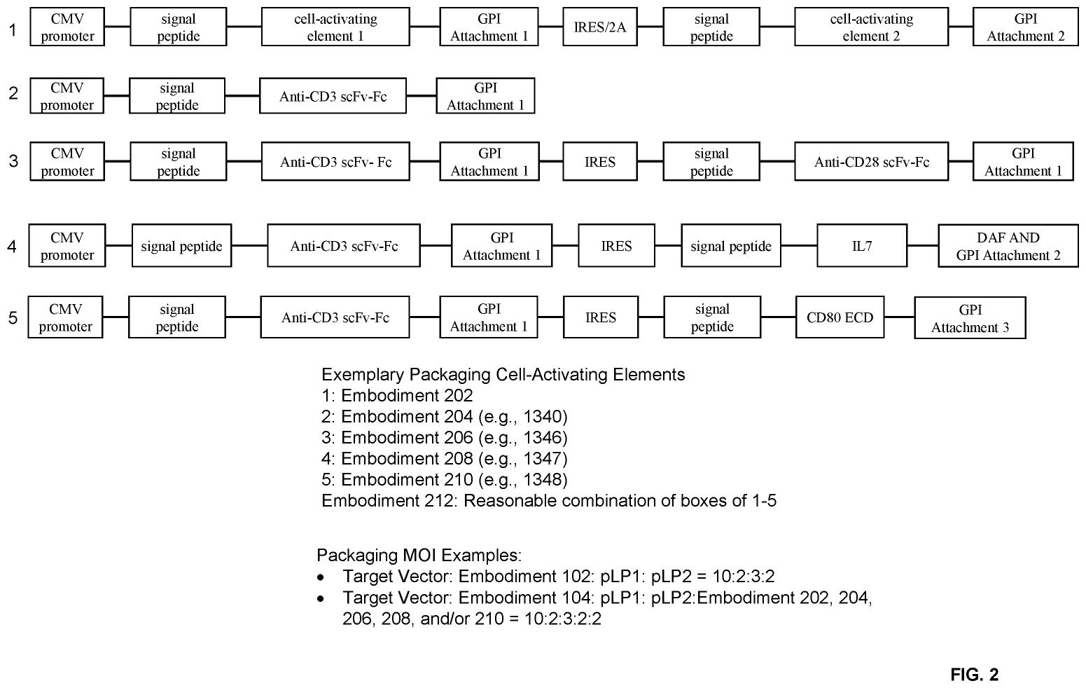

In embodiments, the T cell activating element is included in a packaging-cell activating element. Examples of packaging-cell activating elements include the constructs shown in FIG. 2, such as Embodiments 202, 204, 206, 208, 210, and 212. In embodiments, the packaging-cell activating element includes construct 1340, 1346, 1347, 1348, or a combination thereof. In embodiments, the packaging cellactivating element includes the construct of Embodiment 206 or 1346.

In embodiments, T cell-activating element comprises CD3 and CD28 agonists. In embodiments, these cell-activating elements are membrane bound, for example, to the surface of the virus.

In embodiments, T cell response, T cell expansion in vivo, inhibition growth of tumor cells, a level of modification (e.g., expression ratio) in vivo, and/or treatment of the tumor in a subject administered a composition comprising CD3 and CD28 agonists are enhanced as compared with a subject that is administered with the composition without CD3 and CD28 agonists. In embodiments, the composition comprises an effective amount of protamine sulfate.

In embodiments, T cell response, T cell expansion in vivo, inhibition growth of tumor cells, a level of modification (e.g., expression ratio) in vivo, and/or treatment of the tumor in a subject administered with a composition described herein with protamine sulfate are enhanced as compared with a subject that is administered with the composition without the protamine sulfate.

In embodiments, the blood sample obtained from a subject comprises substantial whole blood, and the blood sample comprises at least two of CD3+ cells, NK cells, myeloid cells, and Neutrophil. In embodiments, the myeloid cells can include a vector encoding IL-12 such that the myeloid cells can overexpress IL-12.

In embodiments, the cell incubation or processing module of a cell therapeutic system is further configured to remove one or more blood cells from the blood sample, and the one or more blood cells comprise B cells. In embodiments, the cell incubation module is further configured to remove one or more blood cells from the blood sample using a bead conjugated with an antibody against a B cell marker.

In embodiments, the blood sample comprises PBMCs.

In embodiments, the one or more viral vectors described herein are in the form of a viral particle that comprises a membrane-bound T cell-activating element on the surface of the viral particle. In embodiments, the membrane-bound T cell-activating element comprises or is anti-CD3. In embodiments, the viral particle further comprises a membrane-bound polypeptide capable of binding to CD28.

In embodiments, the cellular therapeutic system further comprises one or more sensors configured to detect the progress of separation of the blood sample, in particular by detecting the formation of layers of the blood sample, a change of pH value of the blood sample, and/or a change in temperature of the blood sample.

In embodiments, the cell incubation or processing module of the cellular therapeutic system comprises a rotating container configured to culture cells and/or grow cells. The rotating container is disposable and/or has been sterilized.

Embodiments relate to a novel method of CAR T therapy, the method comprising: obtaining a blood sample from a subject, the blood sample comprising substantially whole blood; removing one or more cells, such as B cells, from the blood sample to obtain a group of cells comprising at least CD3+ cells, NK cells, myeloid cells, and Neutrophils; mixing the group of cells with one or more vectors and an agent that activates T cells to introduce the one or more vectors into the group of cells; and infusing at least a portion of the group of cells, introduced with one or more vectors, into the subject.

Embodiments relate to a novel method of CAR T therapy, the method comprising: obtaining a blood sample from a subject, the blood sample comprising substantially whole blood; mixing the blood sample with one or more vectors and an agent that activates T cells to introduce the one or more vectors into cells of the blood sample; removing one or more cells, such as B cells, from the blood sample to obtain a group of cells comprising at least CD3+ cells, NK cells, myeloid cells, and Neutrophils; and infusing at least a portion of the blood sample introduced with one or more vectors to the subject.

In embodiments, a vein-to-vein time for obtaining the blood sample from a subject to infusing at least a portion of the group of cells or blood sample introduced with one or more vectors is between 30 minutes and 1 hour (hr), 1 hr and 72 hours (hrs), 1 hr and 12 hrs, 1 hr and 24 hrs, 1 hr and 48 hrs, 12 hrs and 24 hrs, 12 hr and 48 hrs, or 48 and 72 hrs (including numbers between). In embodiments, the vein-to-vein time is between 30 minutes and 12 hrs or 12 hrs and 24 hrs. In embodiments, the vein-to-vein time is within 12 hrs or 24 hrs.

In embodiments, the one or more vectors comprise a polynucleotide encoding a chimeric antigen receptor (CAR) targeting a WBC antigen and a polynucleotide encoding a CAR targeting a solid tumor antigen.

In embodiments, the one or more vectors further comprise a polynucleotide encoding IL-12, a polynucleotide encoding IL-6, and/or a polynucleotide encoding IFNγ.

In embodiments, the one or more vectors comprise a polynucleotide encoding a CAR targeting a solid tumor antigen, and the solid tumor antigen is a tumor associated MUC1 (tMUC1), PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLECI5, SLC6A3, KISS1R, CLDN18.2, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, B7-H3, or EGFR.

In embodiments, the CAR comprises an antigen binding domain, a transmembrane domain, a co-stimulatory domain, and a CD3 zeta domain. In embodiments, the co-stimulatory domain comprises an intracellular domain of CD27, CD28, 4-1 BB, OX40, CD30, CD40, PD-1, ICOS, lymphocyte function-associated antigen-1 (LFA-1), CD2, CD7, LIGHT, NKG2C, B7-H3, a ligand that binds CD83, or a combination thereof.

In embodiments, the gene and/or cell therapy described herein can be implemented by fusosome techniques. For example, the one or more vectors can be incorporated into fusosomes, which can be used as a vehicle for the delivery of nucleic acids in CAR T therapy. A fusosome comprises a bilayer of amphipathic lipids enclosing a cavity containing a fusogen. The fusosome can be a membrane preparation obtained from a cell. Examples of fusosome include an extracellular vesicle, a microvesicle, a nanovesicle, an exosome, an apoptotic body (from apoptotic cells), a microparticle (obtained from, for example, platelets), an ectosome (obtained from, for example, neutrophiles and monocytes in serum), a prostatosome (obtained from prostate cancer cells), a cardiosome (obtained from cardiac cells), or any combination thereof. Fusosomes can be released naturally from a cell or can be obtained from cells treated to enhance their formation. Fusosomes can also comprise synthetic lipids.

The fusogen enclosed in the cavity of the fusosome can be a fusogen encoded by an endogenous retroviral envelope gene or a fusogen encoded by a pseudotyped vector. Examples of fusogen include a protein, a glycoprotein, a lipid, a small molecule, a virus, or a nucleic acid. In embodiments, the nucleic acid can encode an antibody binding molecule, such as a CAR molecule. The nucleic acid can comprise viral nucleic acid, such as retroviral nucleic acid, for example, a lentiviral nucleic acid. The nucleic acid can comprise nucleic acid of AAV. In embodiments, the fusogen includes therapeutic protein, antibody binding molecule, or a non-mammalian protein, such as a viral protein.

Fusosomes have the desired properties for facilitating the delivery of agents including nucleic acids for gene therapy to a target cell. The agents are the fusogen described herein. In embodiments, the fusosome containing the fusogen can fuse with a target cell. In embodiments, the fusosome. In embodiments, the fusosome containing the fusogen can be administered to a subject in need thereof for treatment of disease, as described herein.

In embodiments, the fusosome described herein comprises one or more fusogens, such as one or more nucleic acids encoding one or more antibody binding molecules, such as CAR molecules. In embodiments, the one or more nucleic acids comprise one or more vectors including the nucleic acid encoding a CAR binding a WBC antigen and the nucleic acid encoding a CAR binding a solid tumor antigen. The vector can comprise viral nucleic acid, such as lentiviral or AAV nucleic acid for delivering the nucleic acid encoding the CAR molecules.

The CAR molecules comprise antigen binding domain that binds WBC and tumor antigens. Examples of WBC antigens and solid tumor antigens are described herein. In embodiments, the WBC antigen is CD19. In embodiments the solid tumor antigen includes tumor associated MUC1 (tMUC1), PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, CLDN18.2, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, B7-H3, or EGFR.

In embodiments, one or more nucleic acids of the fusogen can further comprise one or more vectors including the nucleic acid encoding IL-12, IL-6, and/or IFNγ. The fusosomes described herein can be used with the system or device described herein for delivering one or more nucleic acids of interest to a subject in need thereof, for example, for immune therapy or inducing a T cell response. The fusosome comprising the fusogen can be mixed with the blood cells obtained from the subject for introducing the fusogen into the blood cells so that the transduced blood cells can be reinfused into the subject in need thereof.

In embodiments, the one or more vectors can be modified for decreased cytotoxicity and phagocytosis mediated by PBMCs, evading complement, reducing immunogenicity, and thus improving transduction efficiency. For example, such modifications can include modifications associated with HLA-G or HLA-E to have decreased cytotoxicity mediated by PBMC cell lysis, modifications associated with CD47 can evade macrophage phagocytosis, and/or modifications associated with immunogenic protein knockdown to reduce immunogenicity.

The sequence information is provided in PCT Patent Applications Nos: PCT/CN2016/075061, PCT/CN2018/08891, and PCT/US19/13068, which are incorporated herein by reference in their entirety.

Embodiments describe a polynucleotide comprising a nucleic acid encoding one or more lymphocyte activating elements, such as a T cell-activating element, a nucleic acid encoding protamine, and a nucleic acid encoding a signal peptide. In some embodiments, the signaling peptide is capable of guiding the expression of one or more lymphocyte activating elements on the surface of a recombinant viral particle. In some embodiments, the polynucleotide encoding a signal peptide comprising the MLV-A glycoprotein signal peptide, and/or wherein the polynucleotide further comprises a nucleic acid encoding MLV-A glycoprotein, an anti-CD3 antibody and protamine. In embodiments, the polynucleotide comprises the SEQ ID NO: 50.

Embodiments describe a packaging cell for producing viral particles but not capable of producing replication-competent infectious virus, wherein the packaging cell comprises (1) the polynucleotide described above, (2) one or more polynucleotides encoding one or more helper factors for assembling transduction-competent viral particles, and (3) a gene of interest, such as a gene encoding CAR, associated with a packaging signal. The viral particles are capable of infecting T or NK cells. In embodiments, the packaging cell is the 293T cell line. In embodiments, the packaging cells comprise a lentiviral plasmid containing a gene of interest, for example, Embodiment 5402, a packaging plasmid encoding proteins for entry and integration of viral genomes, and an envelope plasmid encoding envelope proteins.

Embodiments describe a method of increasing viral transduction efficiency, the method comprising: producing the viral particles using the packaging cell described herein; and infecting PBMCs, such as T/NK cells, with the viral particles; wherein the polynucleotide in the viral particles encodes MLV-A glycoprotein and protamine, and wherein the transduction efficiency of the viral particle is greater than that of viral particles using a packaging cell whose env genes encode VSVG glycoprotein and whose polynucleotide does not encode MLV-A glycoprotein or anti-CD3 scFv, and/or viral particles using a packaging cell of which env genes don't include protamine.

Embodiments describe a method of increasing viral transduction efficiency, the method comprising: producing the viral particles using the packaging cell described herein; and infecting blood cells comprising at least T cells and B cells with the viral particles; wherein the polynucleotide comprises a polynucleotide encoding MLV-A glycoprotein, and an amount of infected B cells is lower than that of a packaging cell of which env genes encode VSVG glycoprotein. In embodiments, the B cells are not infected with the viral particles.

In embodiments, the method further comprises: collecting a blood sample from a subject; mixing the blood sample with vectors at a plurality of predetermined multiplicity of infections (MOI); measuring transduction efficacy of the plurality of MOIs; and/or selecting a MOI based on the transduction efficacy and performing transduction of the vectors using the MOI.

In embodiments, a certain amount of virus can infect a fixed number of cells. In embodiments, the method further comprises collecting a blood sample from a patient; dividing the blood sample into cell groups; measuring cell numbers of the cell groups; determining an amount of vectors based on a cell number of individual cell groups; mixing the amount of vectors with the individual cell groups; measuring transduction efficacy of the individual cell groups; selecting a cell group based on the transduction efficacy; determining the amount of vectors and the cell number of the cell group; calculating a ratio of the amount of vectors and the cell number; and/or performing transduction of the vectors using the ratio.

As used herein, the term “multiplicity of infection” or “MOI” refers to the ratio of the number of vectors, for example, lentiviral transduction vectors, to the number of cells being exposed to the vectors. Herein all MOI descriptions, unless otherwise stated, are functional titers, for example, levels of green fluorescent protein (GFP) expression, rather than based on qPCR titers which are about 10-fold higher. The cells are transduced at a MOI of less than 100. In embodiments, the MOI is about 10 to about 99. In embodiment, the MOI is about 0.1 to about 1. In embodiments, the MOI is about 1 to about 10. In embodiments, the MOI is about 10 to about 50. In embodiments, the MOI is about 50 to about 99. In a further embodiment, the MOI is about 50.

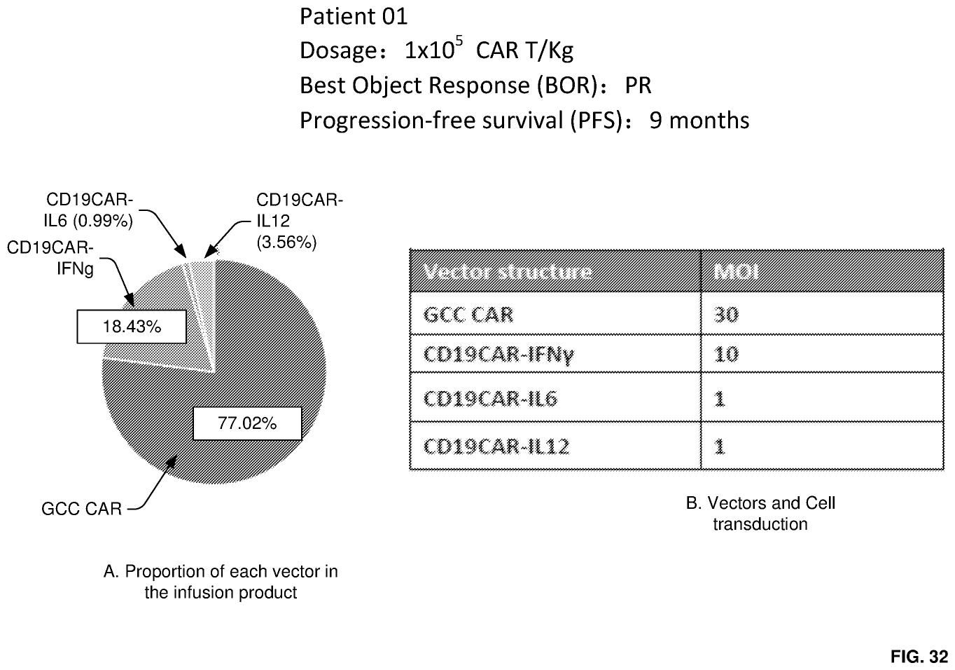

Conventional CAR T cell therapy has emerged as a promising approach for treating cancer, particularly solid tumors. However, there are certain limitations to this approach that need to be considered when treating cancer, especially solid cancer. CAR T cell therapy aims to eliminate cancer cells by reprogramming T cells to attack tumor antigens. This approach is based on the principle of complete or partial elimination of cancer cells, which may be significantly harder than treating other diseases. CAR T technology known as CoupledCAR® has shown promise in treating solid tumors by overcoming certain limitations of conventional CAR T therapy. CoupledCAR CAR T technology has provided antigen-independent expansion of CAR T cells in vitro and in vivo. As shown in Table 3 of the Examples, a unique aspect of CoupledCAR technology is that it can provide 16 different types of cells based on the vector(s) they express. As shown in the Examples and FIGS. 32-36, in patients that responded to CoupledCAR therapy, one specific type of cell is significantly enriched in the patients' blood. This means that the percentage of this specific type of cell is significantly higher in the patient's blood than in the blood containing the CAR T cells infused into the patient. This suggests that this specific type of cell may play a critical role in the effectiveness of the CoupledCAR therapy. On the other hand, in patients that did not show any response to the CoupledCAR therapy, this specific type of cell is not enriched or not significantly enriched in the patients' blood. This suggests that the lack of enrichment of this specific type of cell may be associated with the lack of response to the product.

The discovery that one specific type of cell is significantly enriched in the blood of patients who show a response to the CoupledCAR product is a surprising and significant finding in the field of CAR T cell therapy for solid tumors. This phenomenon highlights the complex dynamics at play in the treatment of solid tumors and the importance of understanding the role of individual cells within the CAR T product. In addition, this phenomenon challenges the traditional understanding of the effectiveness of CAR T cell therapy, has important implications for the selection of patients, and highlights the need for new strategies to enhance the effectiveness of the product.

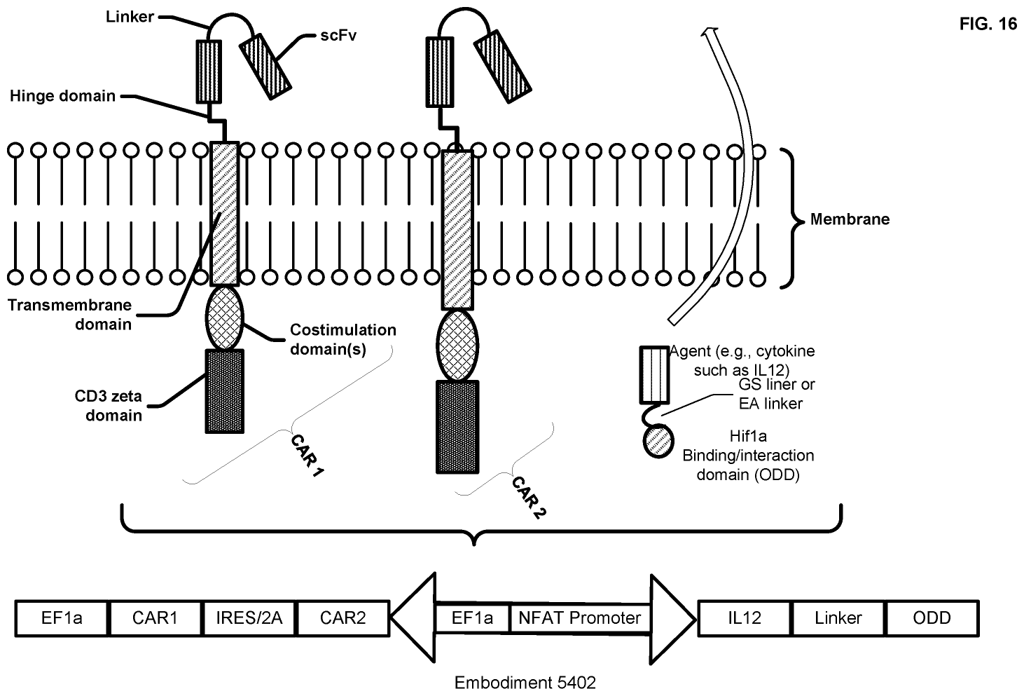

In embodiments, methods and systems described herein can be used to reprogram patients' immune cells to express three polypeptides: (1) a CAR binding a WBC antigen; (2) a CAR binding a solid tumor antigen; and (3) IL-12. An example is Embodiment 5402, as illustrated in FIG. 16. In embodiments, expression vector 2516 can include a polynucleotide encoding a CAR binding a WBC antigen, such as CD19 and BCMA, a polynucleotide encoding a CAR binding a solid tumor antigen, such as GCC and PAP, and a polynucleotide encoding a cytokine, such as IL12. In embodiments, a polynucleotide in the recombinant viral particle comprises a polynucleotide encoding a CAR binding a WBC antigen, for example, CD19 and BCMA, a polynucleotide encoding a CAR binding a solid tumor antigen, for example, GCC and PAP, and a polynucleotide encoding a cytokine, for example, IL12.

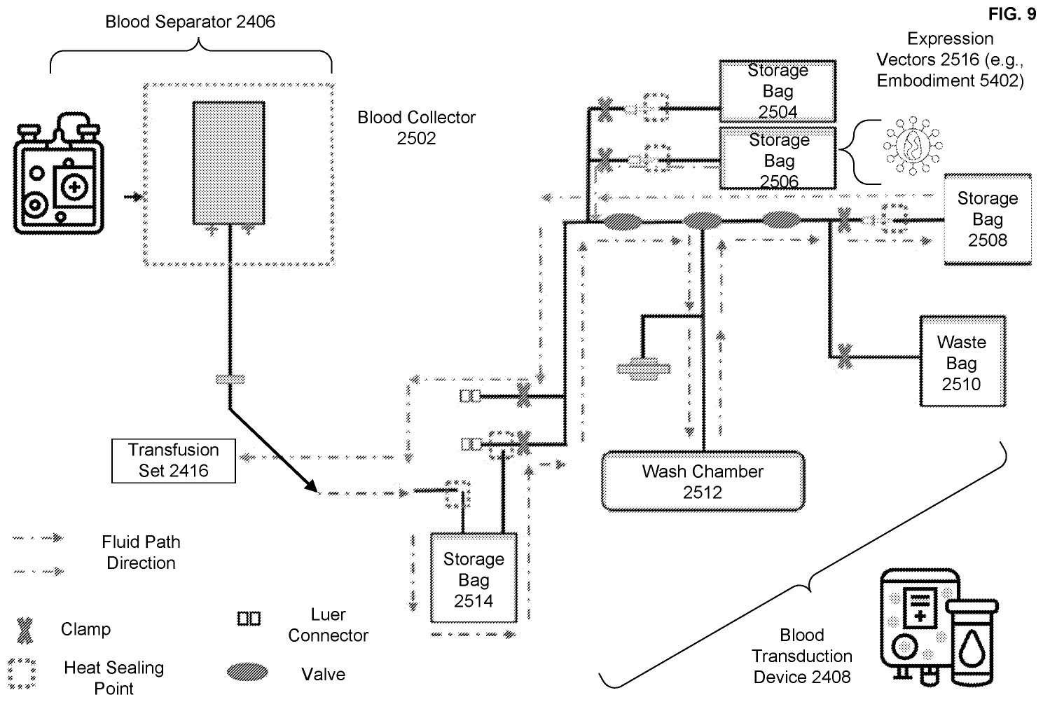

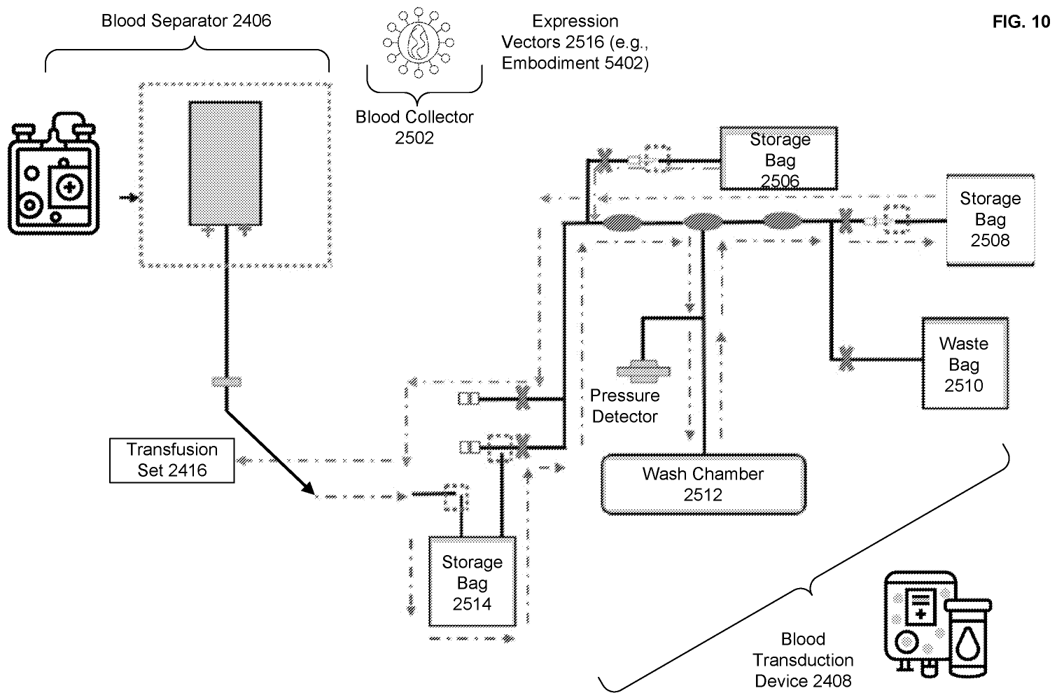

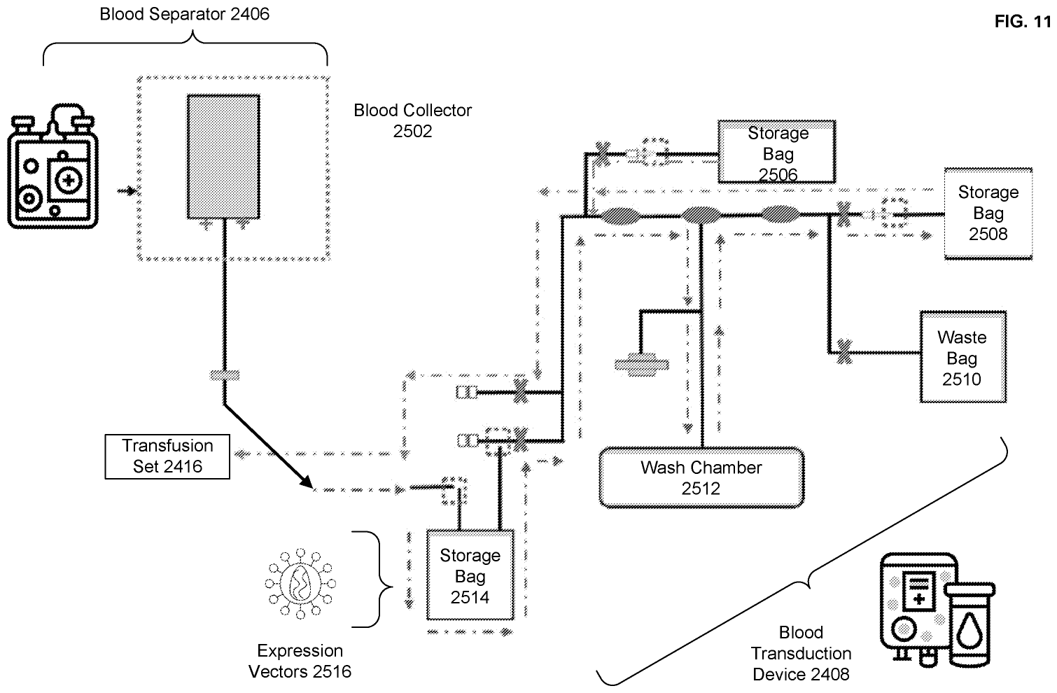

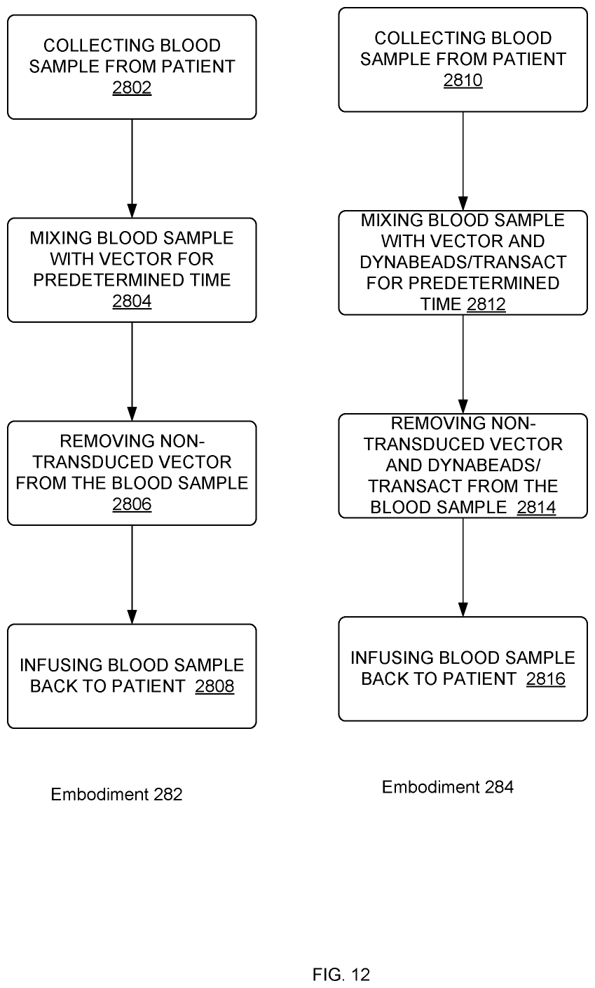

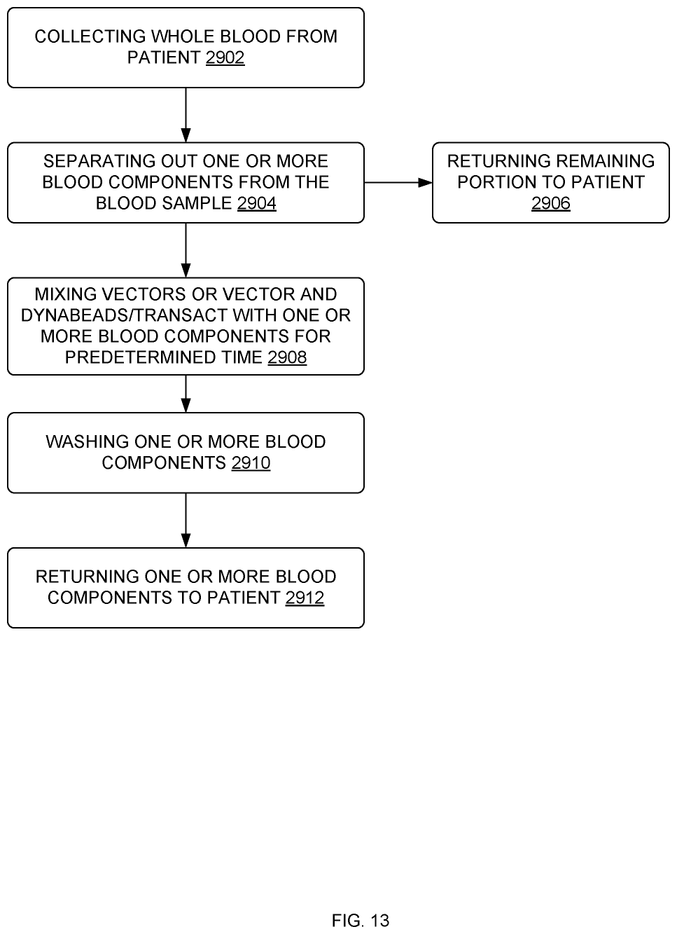

The packaging cell-activating elements are packaging plasmids. In embodiments, the expression vectors are generated using packaging cell-activating elements, vectors 1340 and 1346, the vectors comprising T cell activating elements, CD3 antibodies and/or CD28 antibodies. In embodiments, expression vectors are mixed with protamine sulfate (PS) and PBMCs or the whole blood. In embodiments, expression vectors 2516 are mixed with the whole blood in Blood Separator 2406 (FIG. 8) and/or Blood Collector 2502 (FIG. 9). In embodiments, expression vectors 2516 are mixed with PBMCs in Storage Bag 2506 (FIG. 9). In embodiments, the expression vectors comprise CD3 antibodies and/or CD28 antibodies.

Traditional viral packaging methods rely on the use of a transfer vector, pLPVSVG, pLP1, and pLP2. These methods require either an extended incubation period or a high multiplicity of infection, such as an MOI of 30, in a brief timeframe to generate a significant amount of expression vectors essential for the manufacture of CAR T cells. These conventional methods can be cumbersome, time-consuming, and may increase the risks associated with high MOI. However, an innovative viral packaging approach has been developed that addresses these challenges.

This novel method, surprisingly, demands a lower MOI, for example of less than 10, and a significantly reduced incubation time, such as less than 30 minutes, streamlining the process and reducing potential risks. The key to this breakthrough lies in the integration of cell activating elements in the viral vector, which allows the novel method to effectively tackle two primary obstacles: long times and high MOIs. Prolonged waiting times for cancer patients, mainly due to lengthy cell manufacturing processes, can affect the overall treatment timeline and contribute to patient anxiety and stress. Potential virus leakage resulting from high MOI, which can cause complications and safety concerns for patients receiving CAR T cell therapy.

The expression vectors produced using this innovative viral packaging technique pave the way for the development and implementation of novel CAR technology. This groundbreaking advancement enables CAR T cells to be manufactured and reinfused into patients within just one day, for example in 5-6 hours, significantly reducing the time and cost associated with conventional CAR T cell therapy. This accelerated process has the potential to revolutionize cancer treatment, making it more accessible and affordable while maintaining high levels of safety and efficacy for patients.

The innovative method and novel cellular therapeutic system described in this context offer a groundbreaking approach to CAR T cell therapy, allowing for the rapid production and reinfusion of CAR T cells into patients within a remarkably short period, such as 5-6 hours. This accelerated process has the potential to revolutionize cancer treatment, making it more accessible, efficient, and patient-friendly. The novel cellular therapeutic system streamlines the manufacturing process of CAR T cells by incorporating advanced techniques and technologies that enable faster cell modification, expansion, and purification. In embodiments, it consists of a closed, automated system that reduces the risk of contamination and ensures a controlled environment for cell processing. In other words, the system is configured to operate in a closed and automatic manner. In embodiments, the blood collection, blood component separation, and cell transduction processes occur within the interconnected modules of the system without manual intervention.

The novel cellular therapeutic system described herein can include various modules such including different apparatuses, instruments, devices, or machines. In embodiments, the cellular therapeutic system 2400 comprises a blood exchange module 2404 (e.g., Blood Exchange Kit in FIG. 8), a blood processing module 2406 (e.g., Blood Separator in FIG. 8), and a cell processing module 2408 (e.g., Blood Transduction Device in FIG. 8). The blood exchange module can include one or more kits or apparatuses for drawing blood from the patient and infusing a portion of the blood, for example, the returned blood components after apheresis, and the modified blood back into the patient. The blood exchange module 2404 includes an outlet port connecting to the blood processing module 2406, such that blood drawn from the patient can be flowed from the blood exchange module to the blood processing module 2406. The blood processing module separates out the desired or selected blood cells, such as PBMCs or T cells from the rest of the blood. In embodiments, the blood processing module comprises an apparatus or device that performs apheresis, such as an apheresis machine (blood separator device) and a blood collector 2502. The blood processing module 2406 includes an outlet port connecting to the cell processing module 2408. Optionally, the blood processing module includes another outlet port connecting to the blood exchange module for flowing the rest of the blood to the patient.

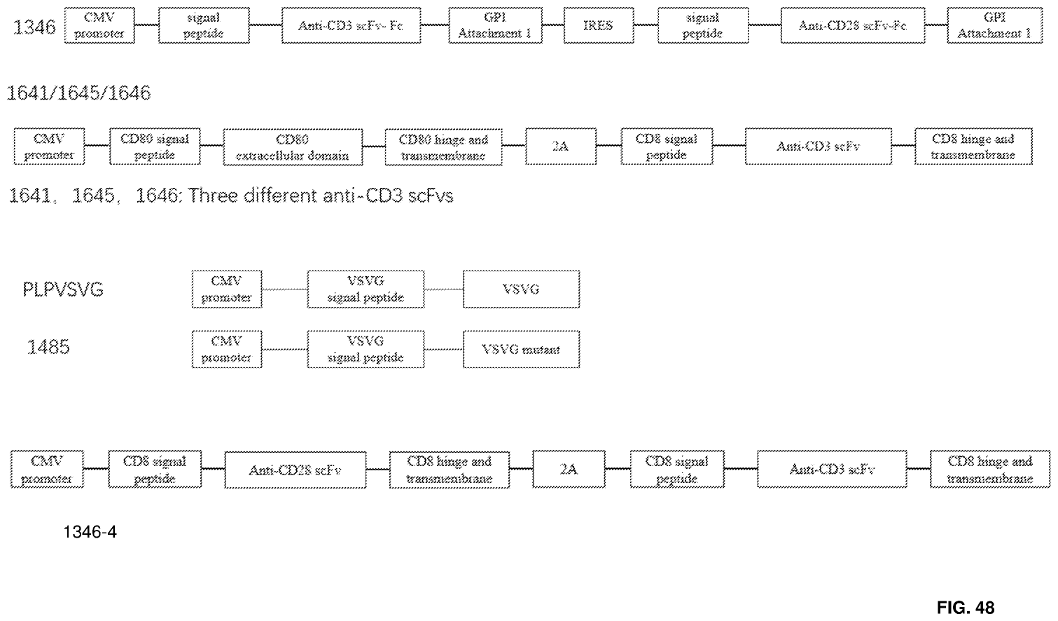

The cell processing module includes a blood transduction device for transducing the desired selected blood cells with vectors or viral particles described herein including for example, vectors or viral particles comprising polynucleotides encoding one or more CAR molecules and/or one or more therapeutic agents, and vectors or viral particles comprising, such as, packaging cell-activating elements of Embodiments 202-212 or constructs illustrated in FIG. 48, which include 1340, 1346,1347, 1348, 1346-4, 1641, 1645, or 1645 and cell activating elements. Exemplary blood transduction device includes the Cytiva GE Sepax C-Pro Cell Processing device. The cell processing module 2408 includes an outlet port connecting to the blood exchange module 2404 for flowing at least a portion of the transduced blood cell to the blood exchange module for infusing into the patient.

The cell processing module 2408 comprising the blood transduction device can further include one or more storage bags 2514, 2504, 2506, and 2508, and waste bag 2510, and wash chamber 2512. Storage bag 2514 stores blood withdrawn from the patient. Storage bag 2514 stores blood cells flowed to the cell processing module from the blood collector 2502 of the blood separator module. Storage bag 2504 and/or 2506 stores the vectors or viral particles. The vectors or viral particles of Storage bag 2506 can be moved to and mixed with the blood cells. Wash chamber 2512 washes the transduced blood cells to remove the non-binding virus and flows the transduced blood cells to storage bag 2508 containing the culture media. At least a portion of the blood cells from storage bag 2508 are flowed or infused into the patient through the blood exchange module 2404. The cell processing module can further include one or more storage bags for storing the infusion media and/or replacing the culture media with the infusion media, such that the transduction or culture media of the transduced blood cells can be replaced with infusion media for infusing the transduced blood into the patient. The cell processing module can also include a device for exchanging the culture media with the infusion media.

Features of the novel cellular therapeutic system include at least one of the following features. (1) Integration of cell activating elements into the viral vector: by incorporating cell activating elements into the packaging cell-activating element used in the method of viral packaging, the system enables faster cell processing, addressing the challenge of prolonged waiting times for cancer patients. (2) Automated cell processing: the closed, automated system minimizes the need for manual intervention, increasing efficiency and reducing potential errors during cell manufacturing. (3) Rapid cell modification and expansion: the system allows for the swift introduction of CAR genes into T cells and supports rapid cell expansion, significantly reducing the time needed for producing a therapeutically relevant number of CAR T cells. (4) Efficient purification and concentration: the system includes optimized steps for cell purification and concentration, ensuring that a high-quality CAR T cell product is obtained in a fraction of the time required by traditional methods. (5) Real-time monitoring: the system offers real-time monitoring capabilities, providing continuous feedback on the progress of cell manufacturing and ensuring that the process remains on track for completion within the accelerated timeframe. By integrating these advanced features, the novel system dramatically shortens the CAR T cell manufacturing process, enabling the production and reinfusion of CAR T cells into patients within just one day, for example, 5-6 hours. This significant reduction in manufacturing time not only benefits patients by allowing for faster treatment but also has the potential to reduce overall treatment costs and increase the availability of CAR T cell therapy for a broader patient population.

In embodiments, the system is configured to operate in a closed and automatic manner, such that the blood collection, blood component separation, and cell transduction processes occur within the interconnected modules of the system without manual intervention. In embodiments, the cellular therapeutic system operates in a closed and automatic manner. For example, this means that the processes of blood collection, blood component separation, and cell transduction all occur within the interconnected modules of the system, eliminating the need for manual intervention.

In embodiments, the automatic operation of the cellular therapeutic system includes programmable control elements. These control elements regulate the timing, duration, and sequence of the blood collection, blood component separation, and cell transduction processes. In embodiments, the closed nature of the cellular therapeutic system ensures sterility by preventing exposure of the blood and selected cells to the external environment. This feature is maintained during all the processes of blood collection, blood component separation, and cell transduction. In embodiments, the closed and automatic system includes feedback mechanisms for monitoring and adjusting parameters during the blood collection, blood component separation, and cell transduction processes. In embodiments, the feedback mechanisms included in the cellular therapeutic system consist of sensors. These sensors are used for detecting real-time information about blood volume, blood cell counts, and the efficiency of cell transduction. Additionally, control elements are included for adjusting the processes based on the information detected by the sensors. In embodiments, the cellular therapeutic system is capable of operating autonomously for a predetermined period. This allows for continuous or intermittent blood collection, blood component separation, and cell transduction processes without the need for manual intervention during the predetermined period. It should be noted that the “closed and automatic” feature as described in the aforementioned embodiments is not limited to those specific embodiments. The description is intended to be illustrative and not restrictive. Variations and modifications are possible without departing from the scope of this invention as defined by the appended claims and equivalents. For instance, the term “automatic” may encompass the automation of one or more procedures within the system, even if not all procedures are automated. This could include, but is not limited to, the processes of blood collection, blood component separation, and/or cell transduction. Similarly, the term “closed” is not limited to a system with just two modules. Systems comprising more than two modules, with each module interconnected to facilitate fluid communication without exposure to the external environment, would also fall within the scope of a “closed” system as per this invention. The specific combination and configuration of procedures and modules can vary based on the requirements of the specific application, and the invention is intended to encompass all such variations and modifications that fall within the scope of the appended claims and their equivalents.

Embodiments relates to a method for improving the proliferation and/or CAR expression of lymphocytes, such as T cells and NK cells, within a specified time frame, which involves generating expression vectors by utilizing a packaging cell line, packaging plasmids, a targeting plasmid such that the expression vectors contain a polynucleotide encoding a single or dual CAR, for example, CD19 CAR and GCC CAR, envelope plasmids, and cell-activating elements1340, 1346, 1519, 1520, or 1521 of Embodiments 202 to 2012; and mixing the expression vectors with a blood sample, for example, blood obtained from apheresis or peripheral blood mononuclear cells (PBMCs), to produce lymphocytes containing the polynucleotide, wherein the proliferation and/or CAR expression is enhanced compared to using envelope plasmids without the cell activating elements or packaging cell-activating elements. In embodiments, the lymphocytes contain the polynucleotide and anti-CD3 and/or anti-CD28 (or CD80 or its extracellular domain) expressed on cell surface. In embodiments, mixing the expression vectors with the blood sample comprises mixing the expression vectors with the blood sample at a MOI, which is not greater than 10, 20, or 30. In embodiments, mixing the expression vectors with the blood sample for less than 0.5, 1, 2, 3, 4, 5, 6, 7, 8, 9, or 10 hours. In embodiments, the cell activating elements comprises polynucleotides encoding anti-CD3 and/or anti-CD28 (or CD80 or its extracellular domain).

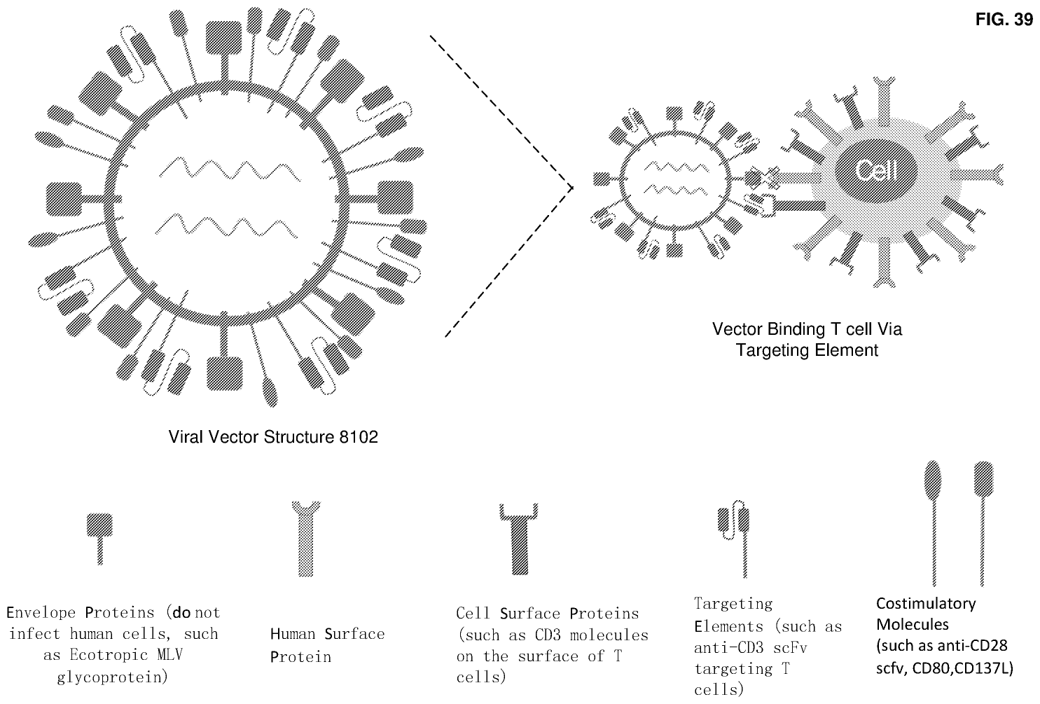

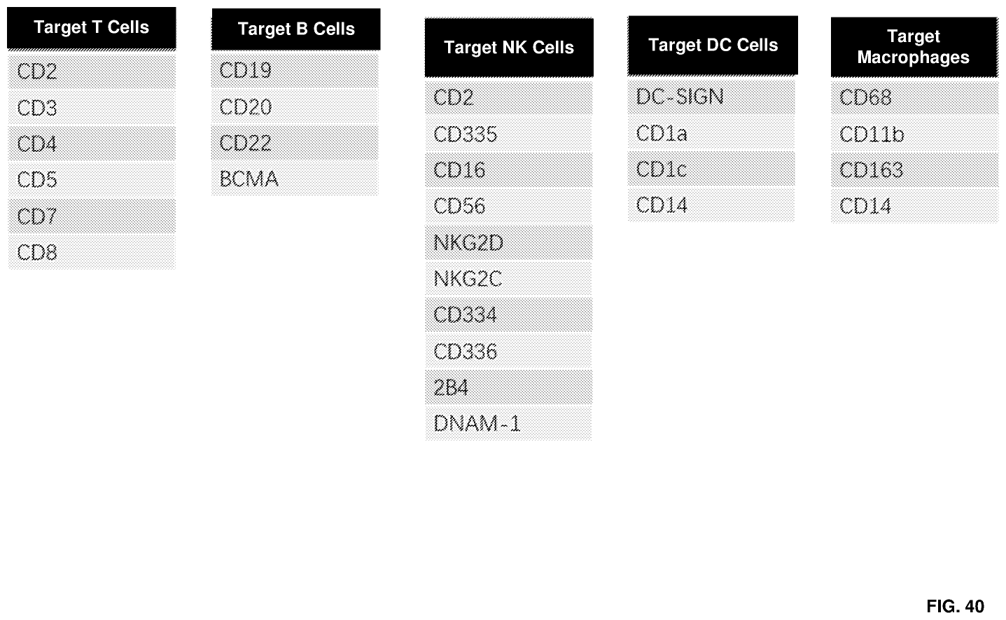

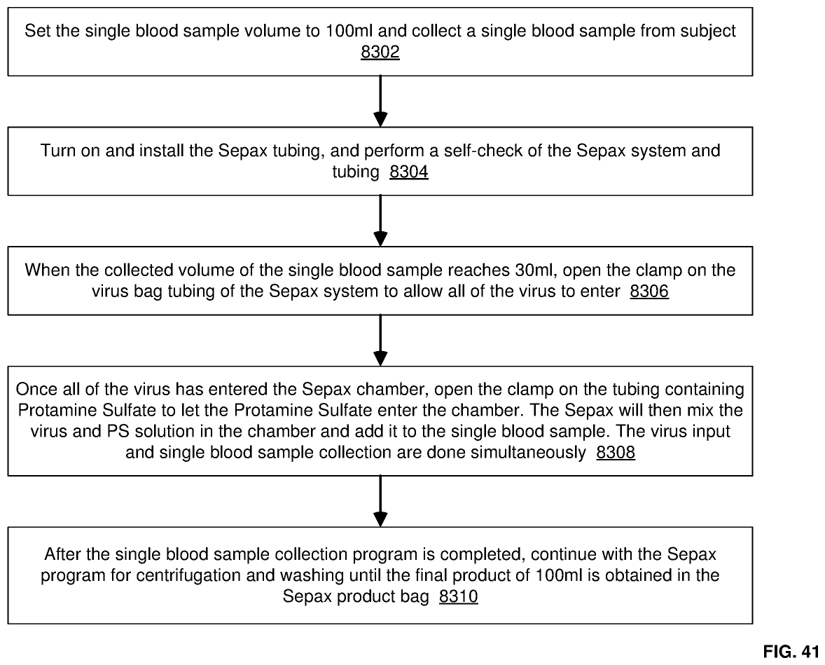

FIGS. 39-41 shows novel design of vectors and their use thereof. CAR technology faces challenges in treating blood tumors and providing direct gene therapy. For instance, VSVG-enveloped lentiviruses or retroviruses can infect tumor B cells in the human body, leading to the creation of CAR-tumor B cells. These CAR-tumor B cells can cause immune escape and tumor recurrence, making them unsuitable for treating hematologic malignancies. Additionally, VSVG-enveloped lentiviruses or retroviruses can infect human tissue cells, such as the brain, liver, muscle cells, posing safety risks when directly injected into the body.

VSVG infects cells by recognizing and binding to the low-density lipoprotein receptor (LDL-R) or other receptors. Some studies have attempted to mutate the binding sites of VSVG and LDL-R to prevent VSVG infection. However, proteins with higher homology in the LDL-R family can still bind to the mutated VSVG, meaning VSVG mutants cannot completely prevent VSVG from infecting cells and are not the ultimate solution.

One potential solution involves using a virus packaged with an envelope that cannot infect human cells. By expressing elements that target immune cells on the virus's surface, the virus can specifically infect immune cells, such as T cells targeted by CD3 antibodies. This approach addresses the issue of VSVG broadly infecting human cells and the safety concerns associated with the direct injection of lentiviruses for in vivo CAR-T therapy. Since this modified virus cannot infect human cells, there is no need to worry about infecting B cells in the blood, making it suitable for treating blood tumors without generating CAR-tumor B cells.

For example, identifying a virus envelope that cannot infect any human cells and modifying it to express one or more targeting elements on the surface can enable specific infection of one or more immune cell types. This modification allows direct in vivo targeting and remodeling of these immune cells. To enhance the targeting and infection capabilities of the virus in vivo, additional modifications to the targeting elements and envelope can be made, such as incorporating T cell co-stimulatory signal elements and cytokine elements. The final application should consider factors like the enveloped virus's half-life in vivo, virus degradation within an appropriate range, and relatively low immunogenicity. In embodiments, the virus envelope can be modified to not bind or bind with low infinity human cells.

In embodiments, the virus envelope can be engineered or altered to eliminate or significantly reduce its affinity for binding to human cells. This modification helps prevent the virus from infecting human cells or binding to them with much lower affinity, thereby increasing the safety and specificity of the viral vector for its intended therapeutic purposes. Such alterations to the virus envelope can be achieved through various genetic engineering techniques, including targeted mutations, deletions, or insertions in the envelope protein-coding regions, or by introducing alternative receptor-binding domains that have a decreased affinity for human cell surface receptors.

By modifying the virus envelope in this manner, the potential off-target effects and safety risks associated with the viral vector can be minimized. This approach can be particularly beneficial for gene therapy applications, where precise targeting and minimal impact on non-target cells are essential for successful treatment outcomes. Furthermore, modifying the virus envelope can also improve the overall performance of viral vectors in delivering therapeutic payloads to specific cell types, such as immune cells, by reducing the likelihood of unintended interactions with other cell types.

Expanding on this concept, additional modifications to the virus envelope can be made to enhance its specificity for the target cell population. For example, incorporating ligands or other targeting moieties that recognize unique surface markers on the desired cell type can further improve the selective targeting and delivery of the viral vector. This strategy can lead to more efficient and safer gene therapy applications, with a reduced risk of off-target effects and adverse events.

In the current preparatory procedures, it has been observed that approximately 0.04 to 0.05% of the viruses persists in the preparation. Despite these viruses being bioengineered to bear CD3 and CD28 antibodies, they maintain the potential to infect non-T cells through the interaction between VSVG and the LDL-R receptor. While the probability of such an infection is minimal, when it does occur, complications could arise. These viruses may insert randomly into the genes of specific cells, which could adversely affect these cells, potentially leading to their malignant transformation.

Embodiments involve the mutation of VSVG to enhance the prevailing viral envelope technology and improve safety. T cells are infected solely via CD3 and CD28 antibodies or CD3 antibodies and CD80 molecules. In this scenario, the potential for the virus to infect other cells via the LDL-R receptor is eliminated or reduced, thus significantly diminishing the risk of complications. As such, VSVG was mutated to remove its binding site with the LDL-R receptor. The results revealed that viruses bearing the VSVG mutant can successfully infect cells solely in the presence of CD3 and CD28 antibodies or CD3 antibodies plus CD80 molecules. The VSVG mutant cannot successfully infect cells without these antibodies or molecules. This dual safeguard greatly reduces the risk of complications.

Based on these results, it was concluded that through the modification of VSVG, the pathway for the virus to infect other cells via the LDL-R receptor has been successfully blocked, thus significantly enhancing the safety of the virus. The associated risk remains insignificant even when reinfusing preparations containing about 0.04% of the virus or even higher quantities. This outcome reduces the risk of complication associated with the current procedures.

At the same time, the structure and expression method of the cell-activating agents can be altered. For example, the transmembrane region of CD8 or CD80 can be utilized to express CD3 and CD28 antibodies or CD80 molecules on the virus surface and anchored on the membrane through a short amino acid sequence (the transmembrane of CD8 or CD80). This innovative design enables the utilization of CD3 and CD28 antibodies, or CD80 molecules and CD3 antibodies, to bind with the virus membrane, enhancing the virus's specificity and safety.

The interaction of VSVG with the LDL-R receptor has been reduced, preventing it from infecting non-T cells. Additionally, modifying the structure and expression of the cell-activating agents ensures that the virus can only infect T cells via CD3 and CD28 antibodies or CD3 antibody and the CD80 molecule. This strategy aims to enhance the safety and specificity of the treatment. Incorporating this dual safeguard helps prevent potential adverse reactions, such as the virus randomly inserting into the genes of other cells. This approach has been designed with precision, intending to ensure the safety of viral preparations.

Embodiments relate to a lentiviral particle that comprises a mutated VSVG envelope protein, which reduces its interaction with the LDL-R receptor. The lentiviral particle additionally incorporates a nucleic acid encoding a CD3 antibody, a CD28 antibody, and/or an extracellular domain of CD80, which are expressible in a T cell. The lentiviral particle also contains a nucleic acid encoding the transmembrane region of CD8 or CD80, allowing the expression of the antibodies and/or the CD80 extracellular domain on the viral surface. In embodiments, the VSVG envelope protein is mutated specifically at sites K47Q and R354A.

In embodiments, the lentiviral particle expresses the CD3 antibody, the CD28 antibody, or the CD3 antibody and the CD80 extracellular domain on its surface.

Embodiments pertain to a lentiviral particle that exhibits limited interaction with the LDL-R receptor on non-T cells.

Embodiments relate to a method for the production of the lentiviral particle. This method introduces the nucleic acids encoding the mutated VSVG envelope protein, the CD3 antibody, the CD28 antibody, and/or the extracellular domain of CD80, and the transmembrane region of CD8 into a host cell. The host cell then expresses these elements, assembling the lentiviral particle, which is harvested.

In embodiments, the method further comprises purifying the lentiviral particles; and contacting the T cells with the lentiviral particle either in vitro or in vivo.

Embodiments relate to a kit comprising the lentiviral particle comprising a packaging cell-activating agent, and instructions for using it in the method of targeting T cells. The packaging cell-activating agent comprises 1340, 1346, 1345, 1641, 1645, and/or 1646.

Embodiments describe a method for using CAR T cells to treat a subject, the method comprising obtaining T cells from a subject, followed by culturing the T cells under conditions that promote their growth. The T cells are subsequently contacted with a lentiviral particle to facilitate their infection, leading to the integration of the nucleic acid into the T cells' genome. In embodiments, the entiviral particles comprise the CD3 antibody and the CD28 antibody, or CD3 antibody and the CD80 extracellular domain, and the infected T cells express one or more CARs. CAR T cells are produced through the method described herein and these CAR T cells can be used for treating diseases and conditions such as cancer.

Embodiments pertain to treating a form of cancer in a patient using a cellular therapeutic system, such as system 2400, to obtain desired blood cells from a patient, process include transducing the desired blood cells to obtain CAR T cells and reintroduce CAR T cells back into the patient. In embodiments, the patient's response to the treatment is monitored through regular checkups or diagnostic assays. The cellular therapeutic system can be configured to carry out the method of treating a patient with cancer, as described herein.

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples but rather should be construed to encompass any and all variations which become evident as a result of the teaching provided herein.

### EXEMPLARY EMBODIMENTS

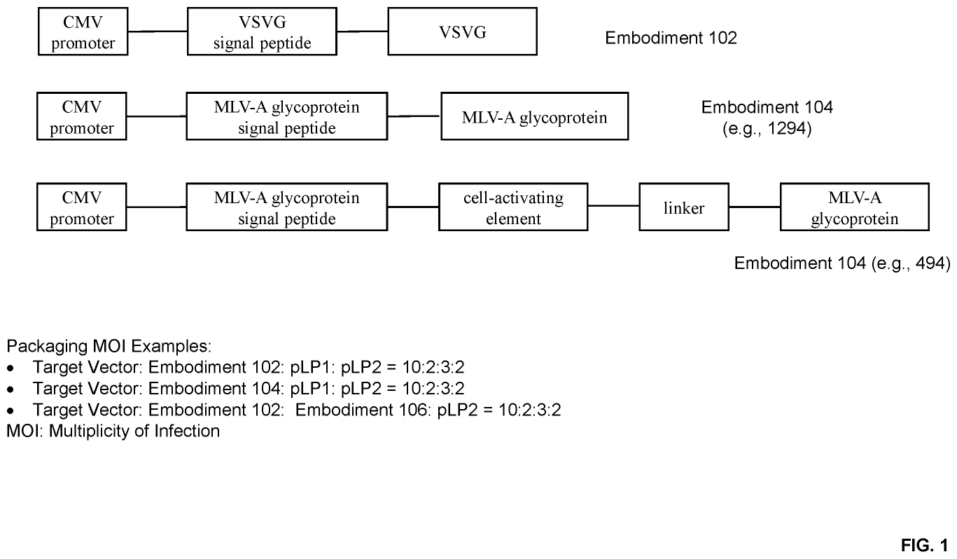

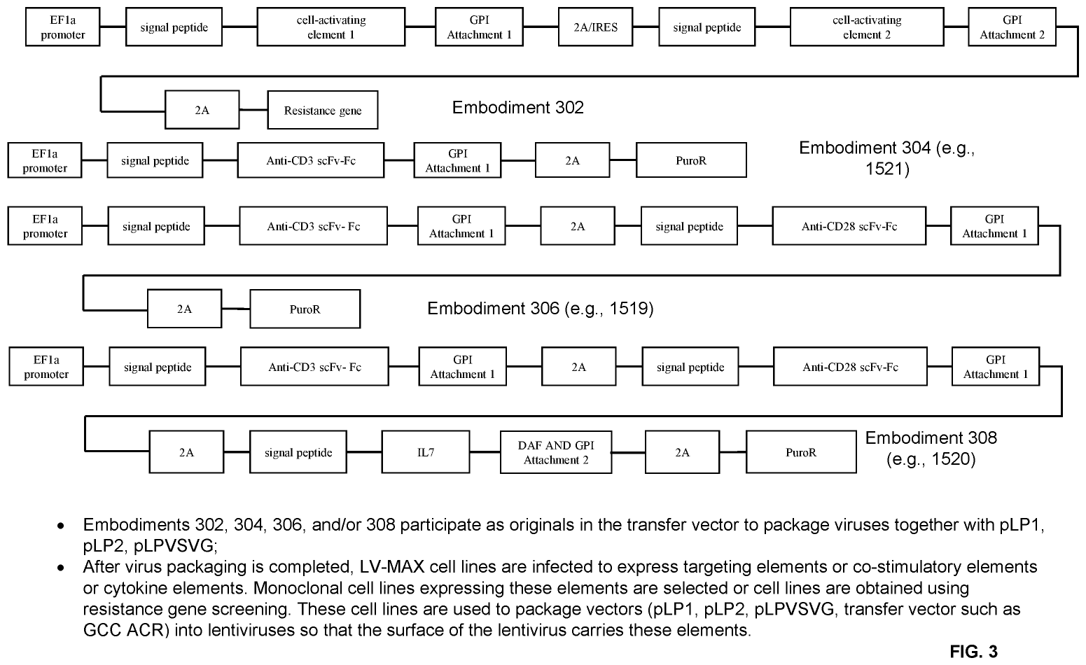

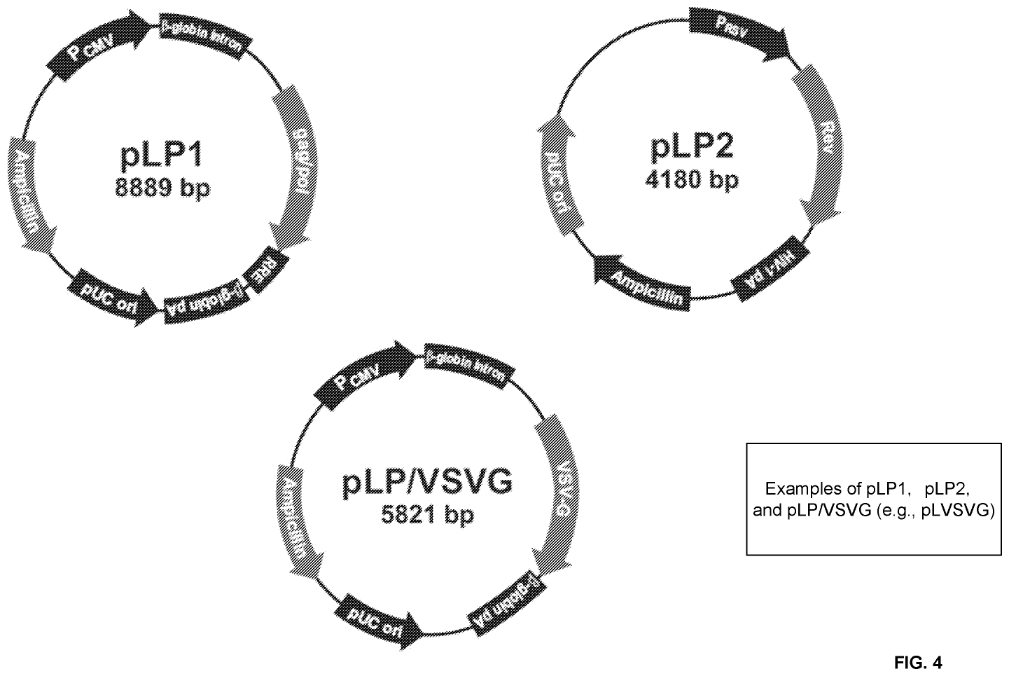

The following are exemplary embodiments:
1. A polynucleotide comprising a polynucleotide encoding one or more lymphocyte activation elements, a polynucleotide encoding protamine, and a polynucleotide encoding a signaling peptide.
2. The polynucleotide of embodiment 1, wherein the signaling peptide is capable of guiding the expression of one or more lymphocyte activation elements on the surface of a recombinant viral particle.
3. The polynucleotide of embodiment 1, wherein the polynucleotide encoding a signaling peptide the signaling peptide comprises MLV-A glycoprotein signal peptide.
4. The polynucleotide of embodiment 3, wherein the polynucleotide further comprises a polynucleotide encoding MLV-A glycoprotein.
5. The polynucleotide of embodiment 1, wherein the polynucleotide comprises the constructs in FIGS. 1-4.
6. A expression system comprising the polynucleotide of any of embodiments 1-5, a pLP1, pLP2, PLVSVG provided in FIGS. 1-4, and package cells.
7. A recombinant viral particle comprising a polynucleotide encoding an antigen binding molecule and the one or more lymphocyte activation elements of any of embodiments 1-5 on the surface of the recombinant viral particle.
8. The recombinant viral particle of embodiment 7, wherein the recombinant viral particle further comprises a pseudo typing element on the surface of the recombinant viral particle.
9. The recombinant viral particle of embodiment 7, wherein the recombinant viral particle is a replication recombinant retroviral particle.
10. The recombinant viral particle of embodiment 7, wherein the recombinant viral particle is a replication recombinant retroviral particle.
11. The recombinant viral particle of embodiment 7, wherein the recombinant viral particle is a lentiviral particle.
12. The recombinant viral particle of any of embodiments 7-11, wherein the one or more lymphocyte activation elements are not encoded by a polynucleotide in the recombinant viral particle.
13. The recombinant viral particle of 12, wherein the recombinant viral particle comprises a polynucleotide encoding an antigen binding molecule or multiple polypeptides (e.g., Embodiment 5402).
14. The recombinant viral particle of 13, wherein the antigen binding molecule is chimeric antigen receptor (CAR), which comprises an antigen binding domain, a transmembrane domain, and an intracellular signaling domain.
15. The recombinant viral particle of 14, wherein the antigen binding domain binds to a tumor antigen is selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, Lewis Y, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin, telomerase, PCTA-1/Galectin 8, MelanA/MART1, Ras mutant, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase (hTERT), RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.
16. The recombinant viral particle of 13, wherein the intracellular signaling domain comprises a co-stimulatory signaling domain, or a primary signaling domain and a co-stimulatory signaling domain, wherein the co-stimulatory signaling domain comprises a functional signaling domain of a protein selected from the group consisting of CD27, CD28, 4-1 BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.
17. The recombinant viral particle of 14, wherein the antigen binding molecule is a modified TCR.
18. The recombinant viral particle of 17, wherein the TCR is derived from spontaneously occurring tumor-specific T cells in patients.
19. The recombinant viral particle of 17, wherein the TCR binds to a tumor antigen.
20. The recombinant viral particle of 17, wherein the tumor antigen comprises CEA, gp100, MART-1, p53, MAGE-A3, or NY-ESO-1.
21. The recombinant viral particle of 17, wherein the TCR comprises TCRγ and TCRδ Chains or TCRα and TCRβ chains, or a combination thereof.
22. One or more recombinant viral particles comprising the recombinant viral particle of any of embodiments 7-21, wherein the one or more recombinant viral particles comprise a polynucleotide encoding a CAR binding a cell surface molecule of a white blood cell (WBC) and a polynucleotide encoding a CAR binding a solid tumor antigen.
23. One or more recombinant viral particles of embodiment 22, wherein the WBC is a granulocyte, a monocyte, or a lymphocyte.
24. One or more recombinant viral particles of embodiment 22, wherein the WBC is a B cell.
25. One or more recombinant viral particles of embodiment 22, wherein the cell surface molecule of the WBC is CD19, CD22, CD20, BCMA, CD5, CD7, CD2, CD16, CD56, CD30, CD14, CD68, CD11b, CD18, CD169, CD1c, CD33, CD38, CD138, or CD13.
26. One or more recombinant viral particles of embodiment 22, wherein the cell surface molecule of the WBC is CD19, CD20, CD22, or BCMA.
27. One or more recombinant viral particles of embodiment 22, wherein the cell surface molecule of the WBC is CD19 or BCMA.
28. One or more recombinant viral particles of embodiment 22, wherein the implementation of the one or more recombinant particles in vivo or ex vivo establishes the CoupledCAR® system described in PCT Publication NOS: WO2020146743 and WO2020106843, wherein the solid tumor CAR and the WBC CAR bind FCRL1, CLDN6, ALPP, GPC-3, and MSLN, wherein the solid tumor CAR comprises at least one scFv listed in Table 2.
29. One or more recombinant viral particles of embodiment 22, wherein the implementation of the one or more recombinant particles in vivo or ex vivo establishes the DoubleCAR® system described in PCT Publication NO: WO2019140100.
30. One or more recombinant viral particles of any of 22-29, wherein the one or more recombinant viral particles comprise a polynucleotide encoding a dominant negative form of a receptor associated with an immune checkpoint inhibitor.
31. One or more recombinant viral particles of embodiment 30, wherein the immune checkpoint inhibitor is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), and CD 160.
32. One or more recombinant viral particles of embodiment 30, wherein immune checkpoint inhibitor is modified PD-1.
33. One or more recombinant viral particles of embodiment 30, wherein the modified PD-1 lacks a functional PD-1 intracellular domain for PD-1 signal transduction, interferes with a pathway between PD-1 of a human T cell of the human cells and PD-L1 of a certain cell, comprises or is a PD-1 extracellular domain or a PD-1 transmembrane domain, or a combination thereof, or a modified PD-1 intracellular domain comprising a substitution or deletion as compared to a wild-type PD-1 intracellular domain, or comprises or is a soluble receptor comprising a PD-1 extracellular domain that binds PD-L1 of a certain cell.
34. One or more recombinant viral particles of embodiment 30, wherein an inhibitory effect of PD-L1 on cytokine production of the human T cells of the population is less than an inhibitory effect of PD-L1 on cytokine production of human T cells that do not comprise at least a part of the nucleic acid sequence that encodes the modified PD-1.
35. One or more recombinant viral particles of embodiment 30, wherein the One or more recombinant viral particles comprise a polynucleotide encoding a therapeutic agent such as a cytokine.
36. One or more recombinant viral particles of embodiment 35, wherein the therapeutic agent that is or comprises IFN-γ.
37. One or more recombinant viral particles of embodiment 35, wherein the therapeutic agent is or comprises at least one of IL-6, IFN-γ, IL-17, and/or CCL19.
38. One or more recombinant viral particles of embodiment 35, wherein the therapeutic agent that is or comprises IL-15 or IL-12, or a combination thereof.
39. One or more recombinant viral particles of embodiment 35, wherein the small protein or the therapeutic agent is or comprises a recombinant or native cytokine.
40. One or more recombinant viral particles of embodiment 35, wherein the therapeutic agent comprises a FC fusion protein associated with a small protein.
41. One or more recombinant viral particles of embodiment 35, wherein the small protein is or comprises IL-12, IL-15, IL-6, or IFN-γ.
42. One or more recombinant viral particles of any of embodiment 35-41, wherein the therapeutic agent is regulated by Hif1a, NFAT, FOXP3, and/or NFkB.
43. One or more recombinant viral particles of any of embodiment 35-42, wherein the one or more recombinant viral particles comprise a polynucleotide encoding hTERT, SV40LT, or a combination thereof.
44. One or more recombinant viral particles of any of embodiment 22-43, wherein the one or more recombinant viral particles comprise a polynucleotide encoding one or more elements of a gene editing tool (ZFN, TALEN, and CRISP).
45. One or more recombinant viral particles of embodiment 44, wherein the gene editing tool cause a cell comprising the one or more recombinant viral particle has a reduced expression of endogenous TRAC gene.
46. One or more recombinant viral particles of any of embodiment 22-45, wherein the one or more recombinant viral particles comprise a polynucleotide encoding a suicide gene such as a an HSV-TK system.
47. One or more recombinant viral particles of any of embodiment 22-46, wherein the One or more recombinant viral particles comprise one or more 2A or /IRES components linked polynucleotides.
48. The recombinant viral particle of any of embodiments 7-47, wherein the lymphocyte is a T or NK cell.
49. The recombinant viral particle embodiment 48, wherein the one or more lymphocyte activation elements comprise anti-CD3 and anti-CD28, and the lymphocyte is a T cell.
50. The recombinant viral particle embodiment 48, wherein the one or more lymphocyte activation elements comprise anti-CD2 and anti-CD335, and the lymphocyte is a NK cell.
51. The polynucleotide or the recombinant viral particle of any of embodiments 1-50, wherein the polynucleotide encodes least one of SEQ ID NO: 1-10.
52. A cell comprises one or more recombinant viral particles of any embodiment of embodiments 1-48.
53. A method of performing or enhancing a gene therapy, the method comprising: administering to a subject having a form of cancer with an effective of a pharmaceutical composition comprising the one or more recombinant viral particles of 7-51; and allowing the viral vector to be introduced into the T or NK cells of the subject.
54. A cellular therapeutic instrument suitable for at least one of collecting a blood sample from a patient having a form of cancer, concentrating cells from a source suspension of the blood sample, transferring vectors to the cells, washing the cells, and returning the transfected cells back to the patient within a predetermined time, the instrument comprising:
   a blood collection apparatus comprising a phlebotomy needle configured to insert into the patient, at least one fluid pump configured to provide fluid flow, and at least one valve configured to control the fluid flow;
   a blood processing apparatus comprising a first rotatable rotor configured to separate one or more blood components from others in the blood sample, at least one fluid pump configured to provide fluid flow, and at least one value configured to control the fluid flow; and
   a cell processing apparatus comprising a blood storage apparatus comprising a medium suitable for transferring vectors to cells of the one or more blood components, a second rotatable rotor configure to separate cells from the vectors, at least one fluid pump configured to provide fluid flow, and at least one valve configured to control the fluid flow; and
   a fluid flow controller comprising a plurality of fluid pumps configured to provide fluid flow and a plurality of valves configured to control the fluid flow, the fluid flow controller configured to:
      receive the blood sample via the phlebotomy needle from the patient,
      transport the blood sample to the blood processing apparatus,
      return the remaining components of the blood sample to the patient via the phlebotomy needle,
      transport the one or more components to the cell processing apparatus; and
      return the one or more components to the patient via the phlebotomy needle.
55. The cellular therapeutic instrument of claim 54, wherein the one or more components are whole blood, Peripheral blood mononuclear cells (PBMCs) or apheresis blood.
56. The cellular therapeutic instrument of claim 54, wherein the vectors comprise a polynucleotide encoding a CAR targeting a solid tumor antigen.
57. The cellular therapeutic instrument of claim 54, wherein the vectors comprise a polynucleotide encoding a CAR targeting a solid tumor antigen (e.g., GCC, PAP, and TSHR) and a polynucleotide encoding CAR targeting a blood cell antigen (e.g., CD19, CD20, and BCMA).
58. The cellular therapeutic instrument of claim 54, wherein the CAR targeting BCMA.
59. The cellular therapeutic instrument of claim 54, wherein the vectors comprise a polynucleotide encoding a chimeric antigen receptor (CAR) targeting CD19 or BCMA and a polynucleotide encoding a therapeutic antigen (e.g., IL-12, IL-6, IL-5, IL-2, and IFNγ).
60. The cellular therapeutic instrument of claim 54, wherein the instrument is a close system when connected to the patient, and at least of two components of the instrument are connected by heat sealing connection.
61. The cellular therapeutic instrument of claim 54, wherein the one or more components comprise peripheral blood mononuclear cells (PBMCs), T cells and NK cells without B cells (e.g., if the CAR binds CD19), or lymphocytes.
62. The cellular therapeutic instrument of claim 61, wherein the B cells are removed using an antibody targeting B cells.
63. The cellular therapeutic instrument of any of claims 54-62, wherein the vectors comprise the polynucleotide of any suitable preceding claim, a recombinant viral particle of any suitable preceding claim, and/or the one or more recombinant viral particles of any suitable preceding claim.
64. A method of inducing or causing an immune response, inhibiting tumor growth, and/or treating a patient having a form of cancer, the method comprising:
   preparing vectors encoding one or more CARs targeting a solid tumor and/or a blood cell antigen;
   placing the medium containing the vectors in the cell processing apparatus of any of claims 54-63;
   connecting the patient with the blood collection apparatus of the cellular therapeutic instrument of any of claims 54-63;
   withdrawing the blood sample from the patient;
   controlling the fluid paths in the cellular therapeutic instrument such that:
      the one or more components are mixed with the medium containing the vectors for less than 15, 30, 45, 60, 90, 120, 150, or 1,440 minutes, preferably 45 minutes,
      the one or more components are returned to the patient via the phlebotomy needle less than 1, 2, 3, 4, 5, 6, 7, 8, 9, 10, 11, or 48 hours after the connecting the patient with the blood collection apparatus, preferably 6 hours.
65. The cellular therapeutic instrument or method of any of claims 54-64, wherein the vectors comprise a polynucleotide encoding a signaling peptide comprises MLV-A or VSVG glycoprotein signal peptide, and/or wherein the polynucleotide further comprises a polynucleotide encoding MLV-A or VSVG glycoprotein, an anti CD3 antibody and protamine.
66. The cellular therapeutic instrument or method of claim 65, wherein the polynucleotide comprises the SEQ ID NO: 50 or 51.
67. The cellular therapeutic instrument or method of any of claims 54-66, wherein the vectors comprise at least one of vectors described in Examples related to FIG. 1-4.
68. The composition or method of any of claims 1-67, wherein the polynucleotide as shown in Embodiment 204and 206 in FIG. 16.
69. The composition or method of any of claims 1-68, wherein the expression vectors are generated using Embodiment polynucleotides listed in FIGS. 1-4.
70. The composition or method of any of claims 1-68, wherein the expression vectors are generated using Embodiments 1340 and 1346.
71. The composition or method of any of claims 1-68, wherein the expression vectors are mixed with PS.
72. The composition or method of any of claims 1-68, wherein one or more packaging plasmids for generating the expression vectors comprise a polynucleotide encoding CD3 antibody and/or a polynucleotide encoding CD28 antibody.
73. The composition or method of any of claims 54-72, wherein the blood sample comprises PBMCs.
74. A method for improving the proliferation and/or CAR expression of lymphocytes (such as T cells and NK cells) within a specified time frame, which involves:
   generating expression vectors by utilizing a packaging cell line, packaging plasmids, a targeting plasmid containing a polynucleotide encoding a single or dual CAR (e.g., CD19 CAR and GCC CAR), envelope plasmids, and cell activating elements (e.g., 1340, 1346, 1519, 1520, or 1521), thereby obtaining the expression vectors; and
   mixing the expression vectors with a blood sample (e.g., apheresis blood or peripheral blood mononuclear cells (PBMCs)) to produce lymphocytes containing the polynucleotide, wherein the proliferation and/or CAR expression is enhanced compared to using envelope plasmids without the cell activating elements.
75. The method of claim 74, wherein the lymphocytes contain the polynucleotide and anti-CD3 and/or anti-CD28 expressed on cell surface.
76. The method of claim 74 or 75, wherein the mixing the expression vectors with the blood sample comprises the mixing the expression vectors with the blood sample at a MOI, which is not greater than 10, 20, or 30.
77. The method of any of claims 74-76, wherein the mixing the expression vectors with the blood sample for less than 0.5, 1, 2, 3, 4, 5, 6, 7, 8, 9, or 10 hours.
78. The method of any of claims 74-77, wherein the cell activating elements comprises polynucleotides encoding anti-CD3 and/or anti-CD28.
79. A closed and automatic cellular therapeutic system for treatment of a subject comprising,
   a blood exchange module for collecting blood from a subject and optionally for infusing blood back into the subject;
   a blood processing module for separating blood components to obtain selected blood cells from the rest of the blood; and
   a cell processing module for transducing the selected blood cells with one or more viral particles comprising one or more cell-activating agents;
wherein,
   the blood exchange module comprises a first outlet port with a tube connecting to the blood processing module for flowing the blood collected from the subject to the blood processing module;
   the blood processing module comprises a first outlet port with a tube connecting to the cell processing module for flowing the selected blood cells to the cell processing module, and optionally, a second outlet port with a tube connecting to the blood exchange module for flowing the rest of the blood to the blood exchange module;
   the cell processing module comprises an optional outlet port with a tube connecting to the blood exchange module for flowing the transduced blood cells to the blood exchange module; and
   optionally, the blood exchange module comprises a second outlet port for infusing the transduced blood cells to the subject, and optionally for infusing the rest of the blood to the subject.
80. The system of embodiment 79, wherein the system completes the treatment of the subject in 30 minutes (mins) to 24 hrs, 30 mins to 5 hrs, 30 mins to 3 hrs, 1 hr to 24 hrs, 2 to 4 hrs, 2 to hrs, 5 to 24 hrs, 5 to 20 hrs, 5 to 16 hrs, 5 to 10 hrs, or 5 to 8 hrs.
81. The system of embodiment 79 or 80, wherein the blood exchange module comprises one or more kits or apparatus for collecting blood from the subject and optionally for infusing blood into the subject.
82. The system of any one of embodiments 79-81, wherein the blood processing module comprises an apparatus for separating blood, and optionally, the apparatus is an apheresis machine.
83. The system of any one of embodiments 79-82, wherein the cell processing module comprises an apparatus for blood transduction.
84. The system of any one of embodiments 79-83, wherein the apparatus for blood transduction comprises one or more storage bags for storing the selected blood cells, storing the one or more viral particles for transduction, mixing the selected blood cells with the one or more viral particles, storing infusion media, and storing the transduced blood cells until ready for use.
85. The system of any one of embodiments 79-84, wherein the apparatus for blood transduction further comprises a wash chamber for washing away non-binding virus particles, and optionally wherein the apparatus for blood transduction further comprises a waste bag for collecting the non-transduced cells.
86. The system of any one of embodiments 79-85, wherein the transduced blood cells are cultured in the bag for storage, and optionally wherein the apparatus for blood transduction further comprises a device for culturing the transduced blood cells.
87. The system of any one of embodiments 79-86, wherein the apparatus for blood transduction further comprises a device for exchanging the culture media of the transduced blood cells with media for infusion.
88. The system of any one of embodiments 79-87, wherein the system further comprises one or more mixers, fluid flow controllers, valves, luer connectors, and/or clamps.
89. The system of any one of embodiments 79-88, wherein the one or more viral particles comprise polynucleotides encoding one or more chimeric antigen receptors (CARs), and optionally wherein the one or more CARs bind a solid tumor antigen.
90. The system of any one of embodiments 79-89, wherein the one or more cell-activating agents comprise a signaling agent 1, a signaling agent 2, and/or a signaling agent 3, and the signaling agent 1 comprises or is a CD3 agonist, the signaling agent 2 comprises or is a CD28 agonist, 4-1BBL, OX40L, CD86, and/or CD80, and the signaling agent 3 comprises or is one or more cytokines, optionally IL7, ILS, IL2, and/or IL12.
91. The system of any one of embodiments 79-90, wherein the CD3 agonist comprises CD3 antibody and the CD28 agonist comprises CD28 antibody, CD86, or CD80.
92. The system of any one of embodiments 79-91, wherein the one or more viral particles comprise SEQ ID NO: 72, SEQ ID NO: 73, SEQ ID NO: 74, SEQ ID NO: 164184, SEQ ID NO: 85, SEQ ID NO: 86, SEQ ID NO: 87, or a combination thereof.
93. The system of any one of embodiments 79-92, wherein the one or more viral particles comprise SEQ ID NO: 88 or SEQ ID NO: 90.
94. The system of any one of embodiments 79-93, wherein the one or more viral particles comprise VSVG, Gag, Pol, Rev, or a combination thereof.
95. The system of any one of embodiments 79-94, wherein the selected blood cells comprise peripheral blood mononuclear cells (PBMCs), and optionally wherein the PBMCs comprise T cells.
96. A viral particle comprising or a polynucleotide encoding SEQ ID NO: 72, SEQ ID NO: 73, SEQ ID NO: 74, SEQ ID NO: 164184, SEQ ID NO: 85, SEQ ID NO: 86, SEQ ID NO: 87 or a combination thereof.
97. A viral particle or a polynucleotide comprising SEQ ID NO: 88 or SEQ ID NO: 90.
99. A composition comprising one or more viral particles of embodiment 96 or 97, and one or more viral particles encoding a CAR and optionally, wherein the CAR binds a solid tumor antigen.
100. A method of treating a subject having cancer comprising obtaining cells generated by the system of any one of embodiments 79-95 or transducing PBMCs with the composition of embodiment 99 to generate cells and administering the generated cells to the subject.
101. A method of generating immunotherapeutic cells comprising obtaining cells generated by the system of any one of embodiments 79-95 or transducing PBMCs with the composition of claim 99 to generate cells and storing the cells.
102. The method of embodiment 100 or 101, wherein treating the subject or generating the immunotherapeutic cells is completed in 30 minutes (mins) to 24 hrs, 30 mins to 5 hrs, 30 mins to 3 hrs, 1 hr to 24 hrs, 2 to 4 hrs, 2 to hrs, 5 to 24 hrs, 5 to 20 hrs, 5 to 16 hrs, 5 to 10 hrs, or 5 to 8 hrs.
103. The method of embodiment 100 or 101, wherein cells are generated with a multiplicity of infection (MOI) of 1 to 10, 2 to 10, 3 to 10, 4 to 10, 5 to 10, 6 to 10, 7 to 10, 8 to 10, 9 to 10, 1 to 8, 1 to 6, 1 to 4, or 1 to 3.

### EXAMPLES

Lentiviral vectors that encode individual CAR molecules were generated and transfected with T cells, which are described below. Techniques related to cell cultures, construction of cytotoxic T lymphocyte assay can be found in “Control of large, established tumor xenografts with genetically retargeted human T cells containing CD28 and CD137 domains,” PNAS, Mar. 3, 2009, vol. 106 no. 9, 3360-3365 and “Chimeric Receptors Containing CD137 Signal Transduction Domains Mediate Enhanced Survival of T Cells and Increased Antileukemic Efficacy In Vivo,” Molecular Therapy, August 2009, vol. 17 no. 8,1453-1464, which are incorporated herein by reference in its entirety.

### Example 1: Novel CAR Technology

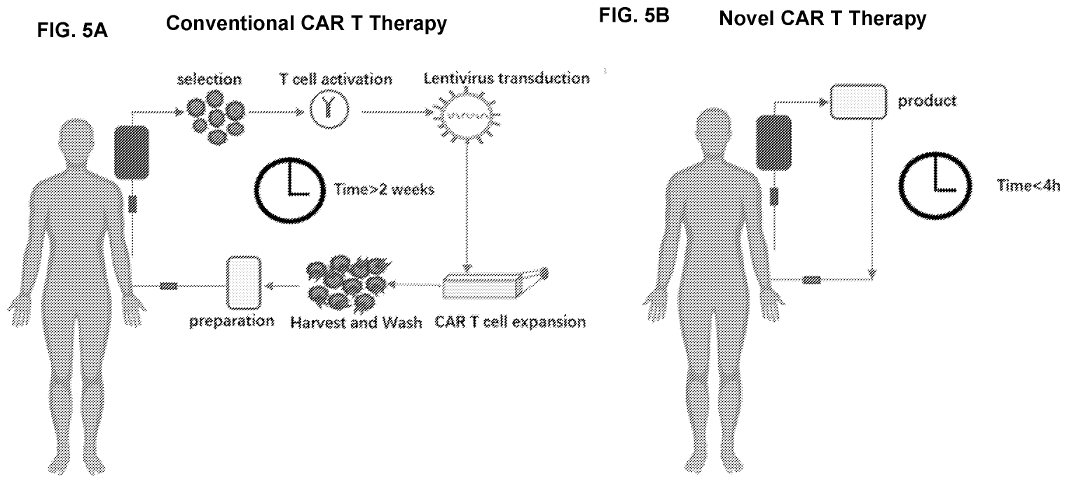

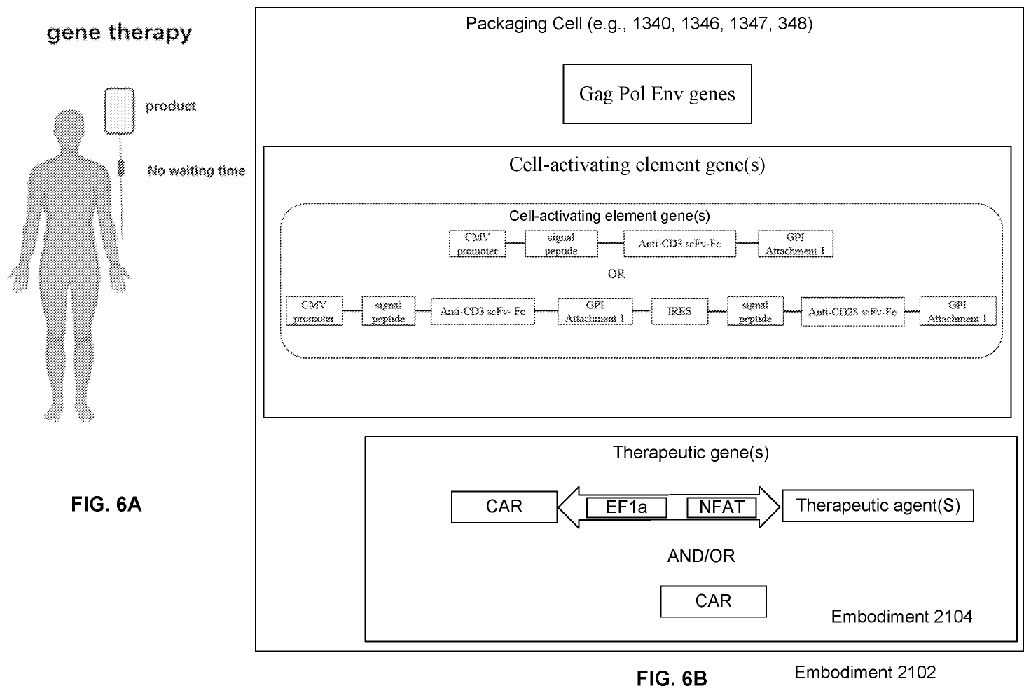

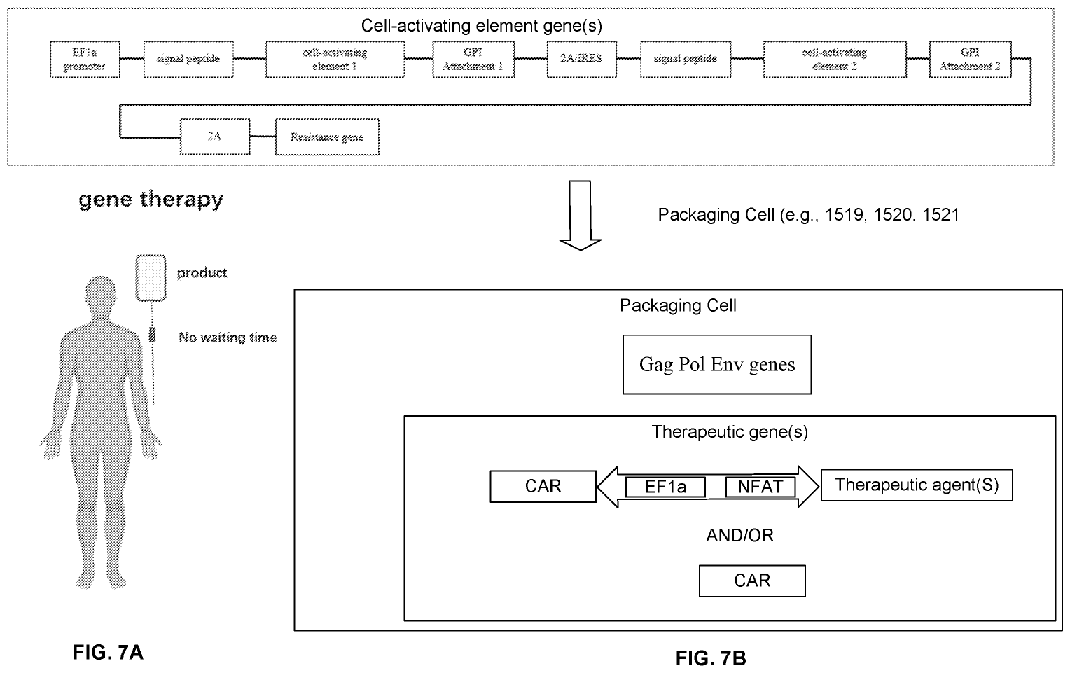

FIGS. 5A and 5B compare conventional CAR T therapy and novel CAR T therapy. Primary T cells were transduced with lentiviral vectors (see table below) to generate mixed CAR T cells. The primary T cells were obtained from healthy human donors. The lentiviral vectors included nucleic acids encoding CAR molecules and one or more therapeutic agents, and can further include the IRES-mCherry construct, which encodes red fluorescence for confirmation of CAR expression. Techniques related to cell cultures, construction of lentiviral vectors, and flow cytometry may be found in “Treatment of Advanced Leukemia in Mice with mRNA-Engineered T Cells, HUMAN GENE THERAPY 22:1575-1586 (December 2011)”, which is incorporated herein by reference. After the transduction, mixed T cells were analyzed to determine individual cell types and percentages.

Each type of CAR T cells were co-cultured with its respective antigen-expressing cells, K562 cells, and the response of the CAR T cells induced by the antigen-expressing cells were measured. CAR T cells (effector (E) cells) and tumor cells (target (T) cells) were co-cultured for 24 hours at a ratio of E:T of 3:1. The supernatant was then collected, and the release of IFN-γ was measured. Various levels of IFN-γ release were observed when CAR T cells and their corresponding antigen-expressing K562 cells were co-cultured.

Assays were conducted to measure the effectiveness of CAR T cells in killing tumor cells. Primary T cells were obtained from blood samples of healthy human donors. These primary T cells were transduced with a nucleic acid encoding various CARs, and CAR expression on T cells was measured using flow cytometry techniques.

Table 3 shows the different types of cells based on the vectors they express. Table 4 shows the various CAR T cells.

TABLE 3
Vector MOI and Estimates of Transduction Rates

| | Construct of CAR | | |
|---|---|---|---|
| | and Therapeutic | Theoretical | Estimated |
| | Agent or dnPD-1 (if | single- | single- |
| Vector | not specified, costimulatory | turn | turn |
| MOI | domain is 41-BB) | ratio | ratio |
| Group 1 | | | |
| 30 | scFv/GCC, | 30.00% | 15.00% |
| 10 | scFv/CD19- IFNγ, | 10.00% | 5.00% |
| 1 | scFv/CD19-IL6, | 1.00% | 0.50% |
| 1 | scFv/CD19-IL12, | 1.00% | 0.50% |
| Group 2 | | | |
| 10 | scFv/PAP-CD28- IFNγ, | 10.00% | 5.00% |
| 10 | scFv/PAP-IL12, | 10.00% | 5.00% |
| 10 | scFv/PAP-dnPD1-IL6 | 10.00% | 5.00% |
| 10 | scFv/CD19- IFNγ, | 10.00% | 5.00% |
| Group 3 | | | |
| 10 | scFv/PAP-CD28- IFNγ | 10.00% | 5.00% |
| 10 | scFv/PAP-IL12, | 10.00% | 5.00% |
| 10 | scFv/PAP-dnPD1-IL6 | 10.00% | 5.00% |
| 20 | scFv/CD19- IFNγ, | 20.00% | 10.00% |

TABLE 4
Measured Transduction Rates for Group 1

| | | |
|---|---|---|
| GCC+ | 17.39% | 17.88% |
| CD19 + IFNγ+ | 4.64% | 2.21% |
| CD19 + IL6+ | 0.05% | 0.09% |
| CD19 + IL12+ | 0.28% | 0.20% |
| GCC + CD19 + IFNγ+ | 4.17% | 2.47% |
| GCC + CD19 + IL6+ | 0.08% | 0.06% |
| GCC + CD19 + IL12+ | 0.33% | 0.09% |
| CD19 + IFNγ + IL6+ | 0.07% | 0% |
| CD19 + IFNγ + IL12+ | 0.03% | 0.02% |
| CD19 + IL6 + IL12+ | 0% | 0% |
| GCC + CD19 + IFNγ + IL6+ | 0.15% | 0.06% |
| GCC + CD19 + IFNγ + IL12+ | 0.04% | 0.05% |
| GCC + CD19 + IL6 + IL12+ | 0% | 0% |
| CD19 + IFNγ + IL6 + IL12+ | 0.05% | 0.03% |
| GCC + CD19 + IFNγ + IL6 + IL12+ | 0.01% | 0.02% |
| CD19+, no vector-cytokine | 3.07% | 2.59% |
| GCC + CD19+, no vector cytokine | 2.68% | 1.58% |
| Un-transduced T | 66.95% | 72.64% |
| Summary | | |
| GCC+ | 24.85% | 22.21% |
| CD19+ | 12.97% | 7.89% |
| GCC + CD19+ | 7.46% | 4.33% |

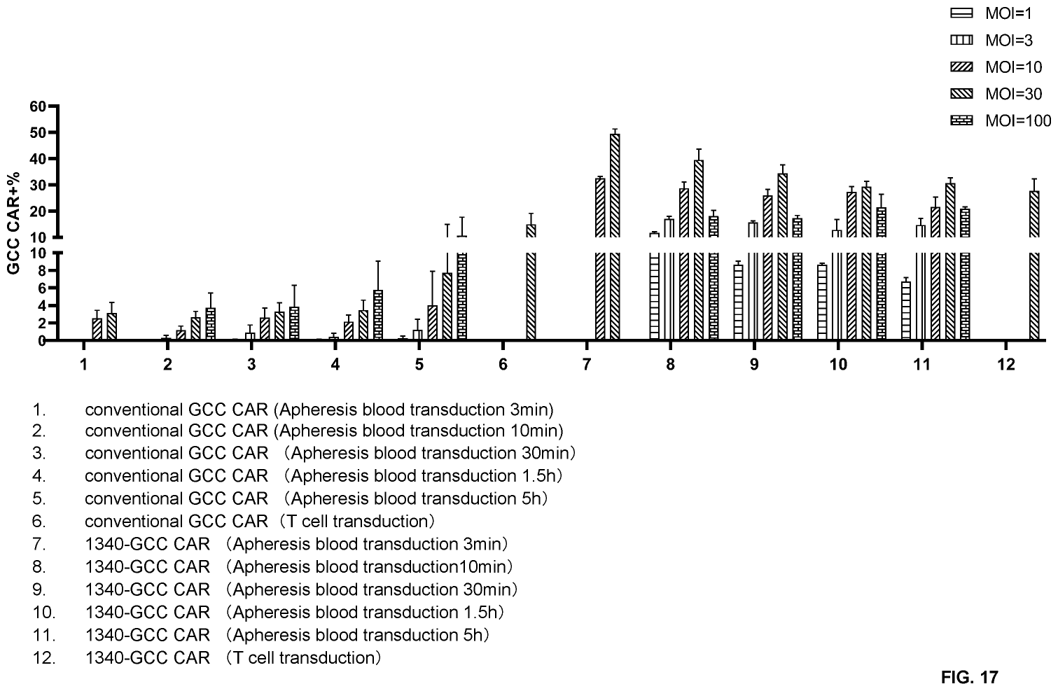

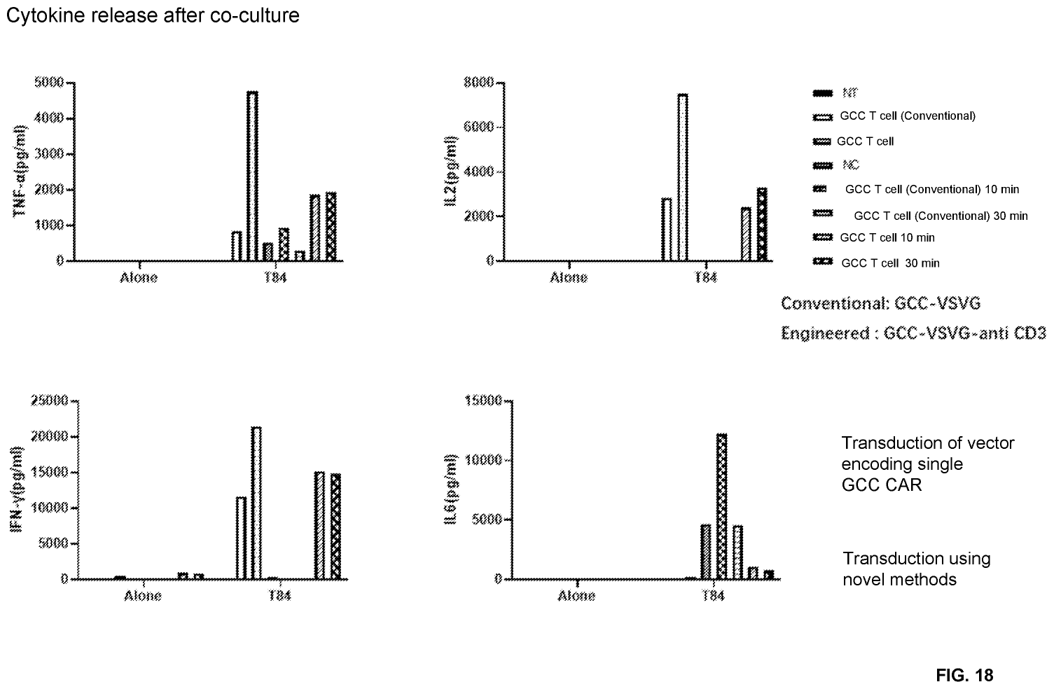

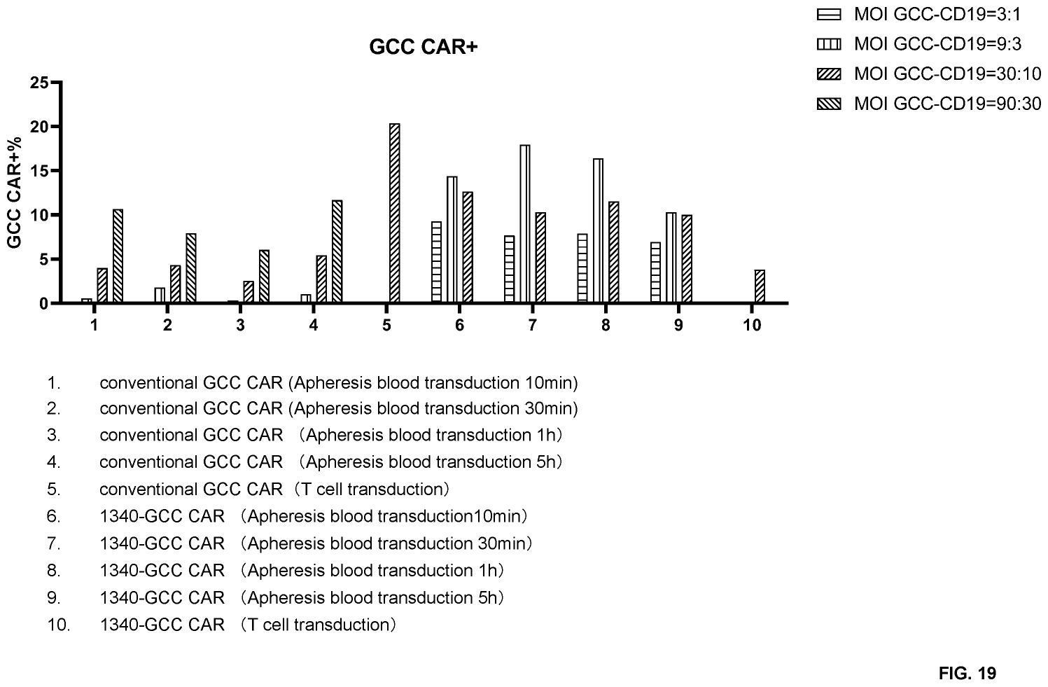

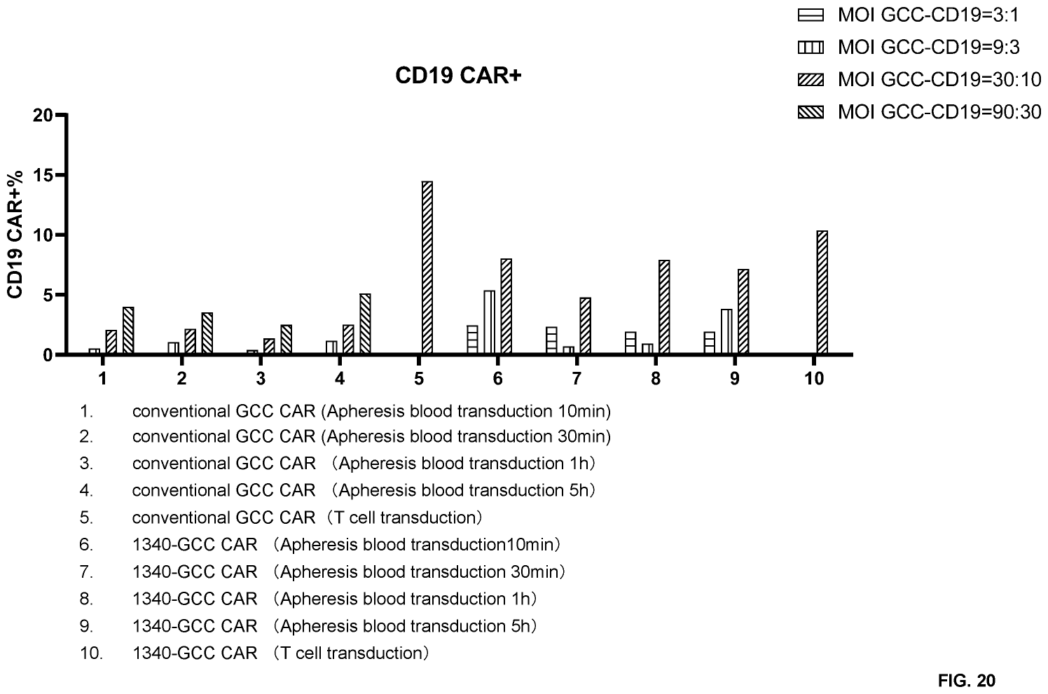

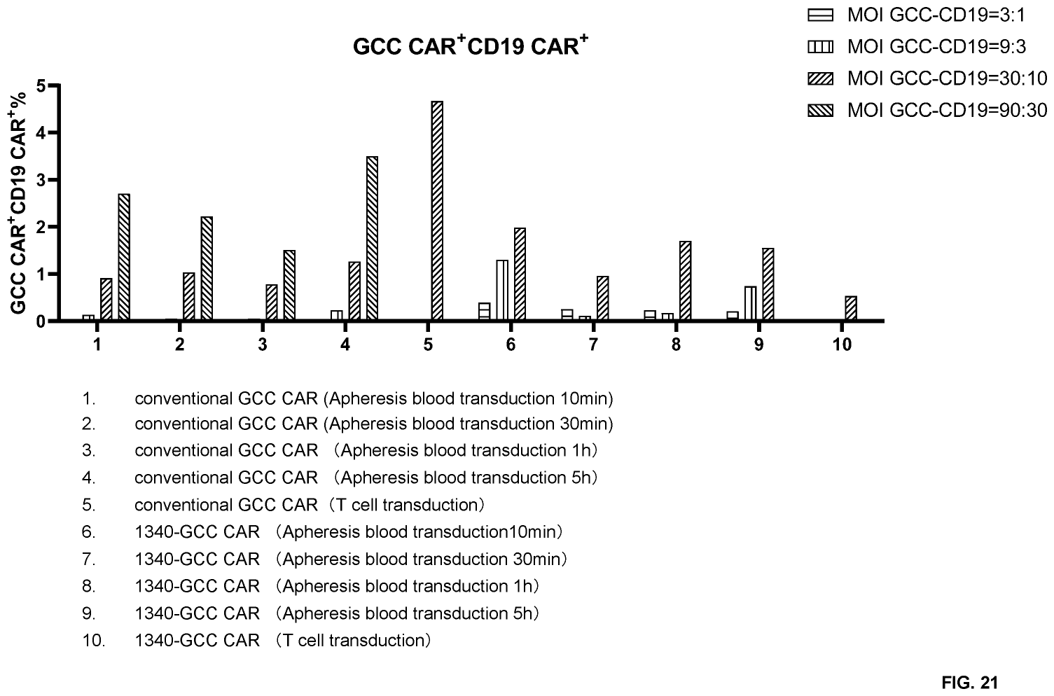

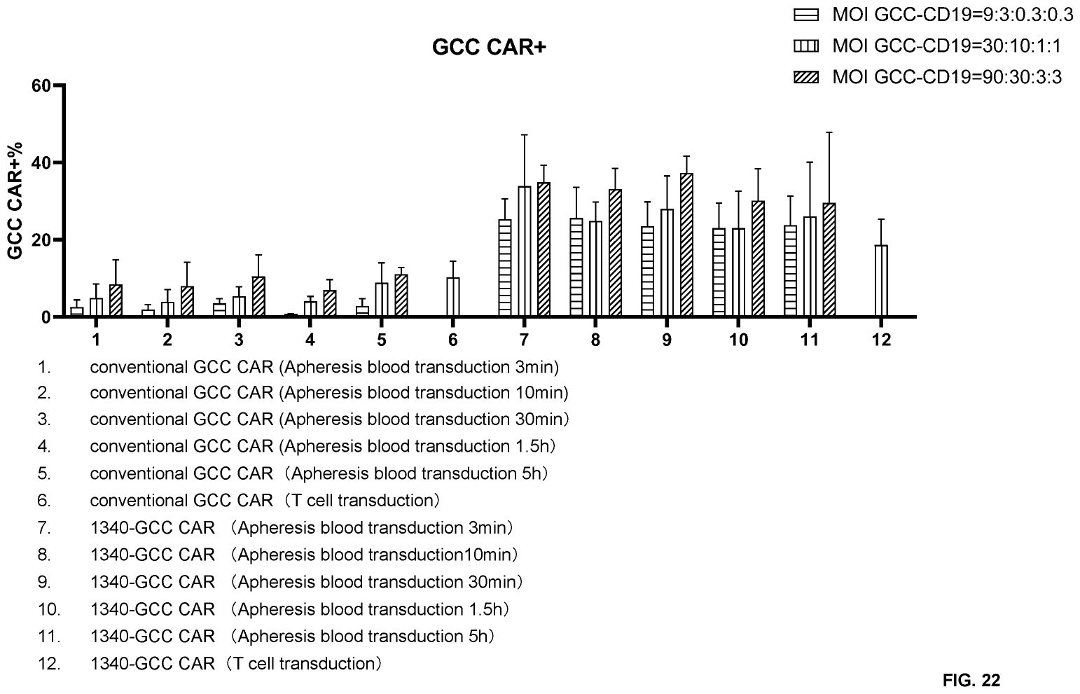

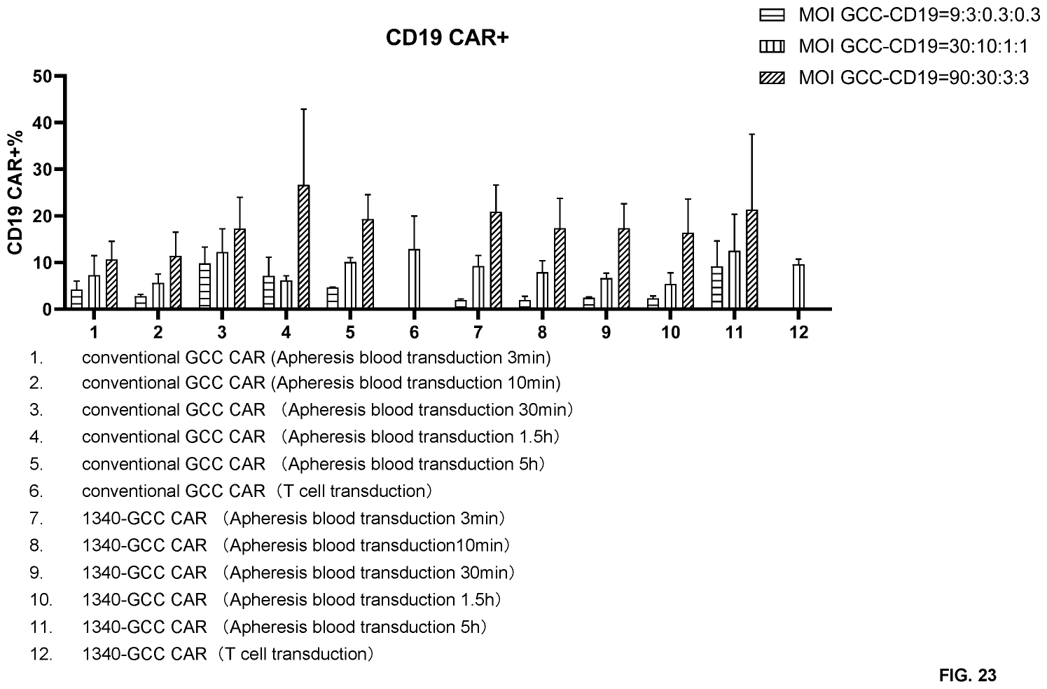

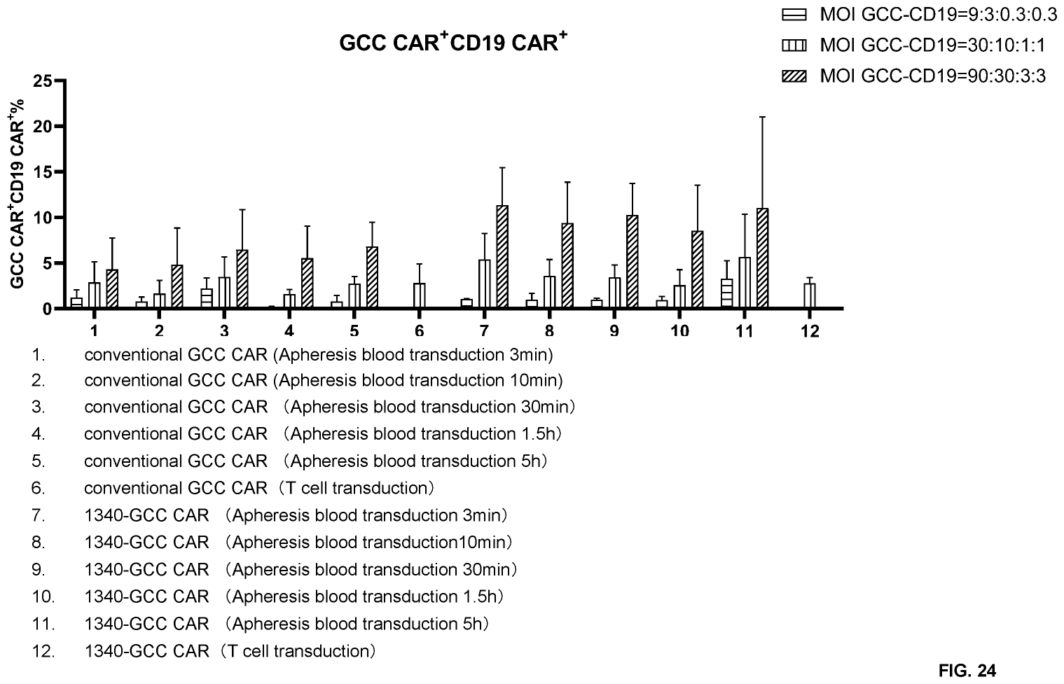

FIGS. 17-24 show histograms of flow cytometry results of GCC CAR (single lentiviral vector) expression as well as CD19 CAR and GCC CAR (two separate vectors encoding two different CARs, coupled CAR) expression in T cells using conventional and novel methods described therein.

As shown in these figures, the efficiency of GCC CAR transduction (single lentiviral vector) and coupled CAR transduction (two separate vectors) using conventional methods is low under high or low multiplicity of infection (MOI) conditions, while the efficiency of these transduction using the novel methods is significantly higher than those using conventional methods. Further, from the perspective of infection time, the expression efficiency of the transduction of GCC CAR did not show significant differences with the change of infection time, which can greatly shorten the infection time. Moreover, GCC CAR T cells generated using novel methods released more cytokines as compared to those generated using conventional methods.

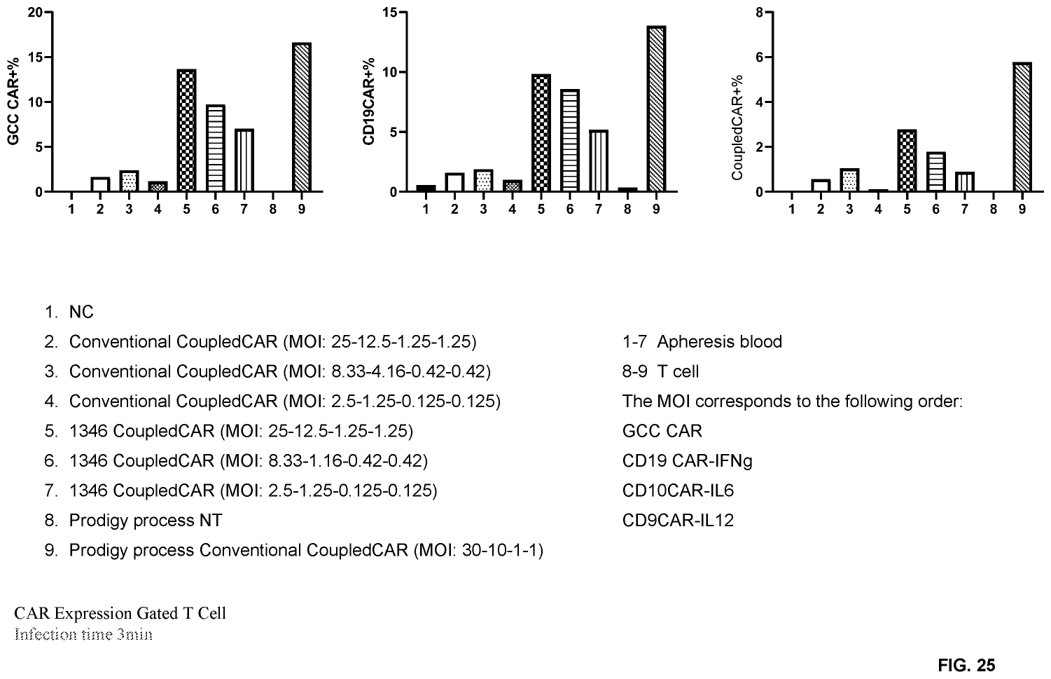

FIG. 25 shows comparisons of CAR expression of CAR T cells generated using conventional techniques and novel methods. Modified transduction refers to the transduction of the vectors with the packaging cell-activating element shown for example in FIG. 2. Unmodified transduction refers to the transduction of the vectors without the packaging cell-activating element. (1) After modified transduction of four vectors (GCC CAR, CD19 CAR-IFN-γ, CD19 CAR-IL-6, and CD19 CAR IL-12) with packaging cell-activating element 1346 (FIG. 2, Embodiment 206) into blood cells, single blood collection showed that the expression of GCC CAR or CD19 CAR reached more than 5% under lower MOI (2.5-1.25-0.125-0.125) conditions. In contrast, the unmodified transduction of the four vectors without the packaging cell-activating element into blood cells, only provided less than 2% expression of GCC CAR or CD19 CAR even at a higher MOI (25-12.5-1.25-1.25). (2) After modified transduction of the four vectors with 1346, there is about 3% double-positive expression of GCC CAR and CD19 CAR under high MOI (2.5-1.25-0.125-0.125) conditions, and about 1% double-positive expression (coupled expression) of GCC CAR and CD19 CAR under low MOI (2.5-1.25-0.125-0.125) conditions. In contrast, the unmodified transduction of the four vectors at the high MOI (25-12.5-1.25-1) had less than 1% double-positive expression of GCC CAR and CD19 CAR, and about 0% double-positive expression of GCC CAR and CD19 CAR under the low MOI (2.5-1.25-0.125-0.125) conditions. (3) Under the conditions of low MOI (2.5-1.25-0.125-0.125), after modified transduction of four vectors with 1346, the expressions for GCC CAR, CD19CAR, GCC and CD19 double-CAR are close to those in Prodigy Process Conventional Coupled CAR (MOI:30-10-1-1). In contrast, the expressions of the CAR molecules for the unmodified transduction of the four vectors are much lower than those in Prodigy Process Conventional Coupled CAR (MOI:30-10-1-1). (4) Modified transduction using 1346 have a higher efficiency in infecting T cells from apheresis blood collection samples.

In the figures, “NT” is “non-transduced,” and “NC” is “negative control” (apheresis blood).

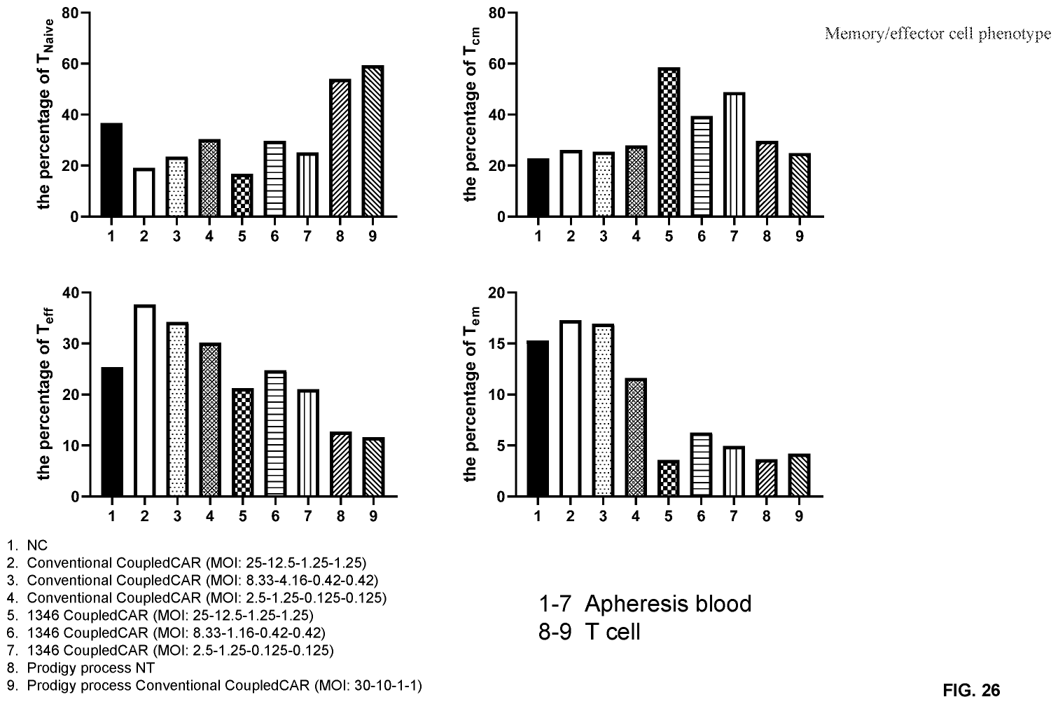

FIG. 26 shows histograms comparing the expression of different types of CAR T cells generated using conventional techniques and novel methods. (1) Compared with the conventional Coupled CAR T cells produced by Prodigy technology, modified transduction of the CAR vectors with 1346 of apheresis blood T cells produce more Teffector (Teff) cells (5%-9% more), less Tnaive (Tn) (5%-8% less), and more T central memory (Tcm) (5%-15% more). (2) The phenotype of T cells in apheresis blood may be affected by blood and other cell factors, resulting in a slight decrease in Tn and a partial increase in Teff and Tcm.

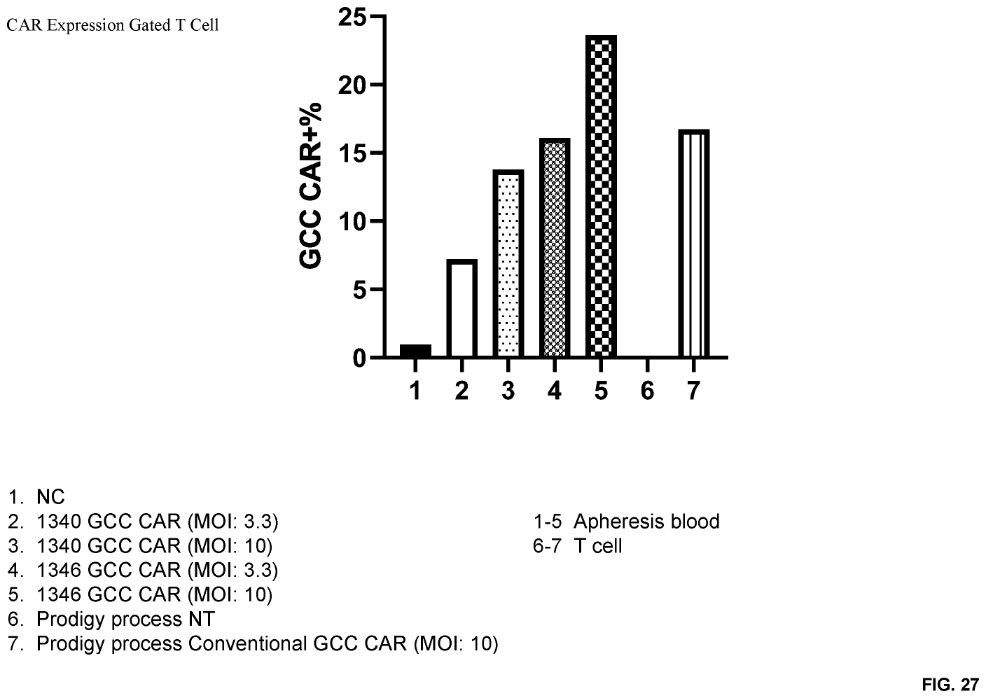

FIG. 27 shows a histogram comparing the expression of GCC CAR in gated T cells generated using various methods. GCC CAR vector with packaging activating cell element, 1340 or 1346, can transduce T cells in apheresis blood samples, and the transduction efficiency increased with the increased MOI. When MOI is 10, the transduction efficiency of 1340 reached that of Prodigy Process Conventional GCC CAR. The transduction efficiency of 1346 is higher than that of 1340, which may be due to the CD28 antibody of 1346, providing a stronger activation signal.

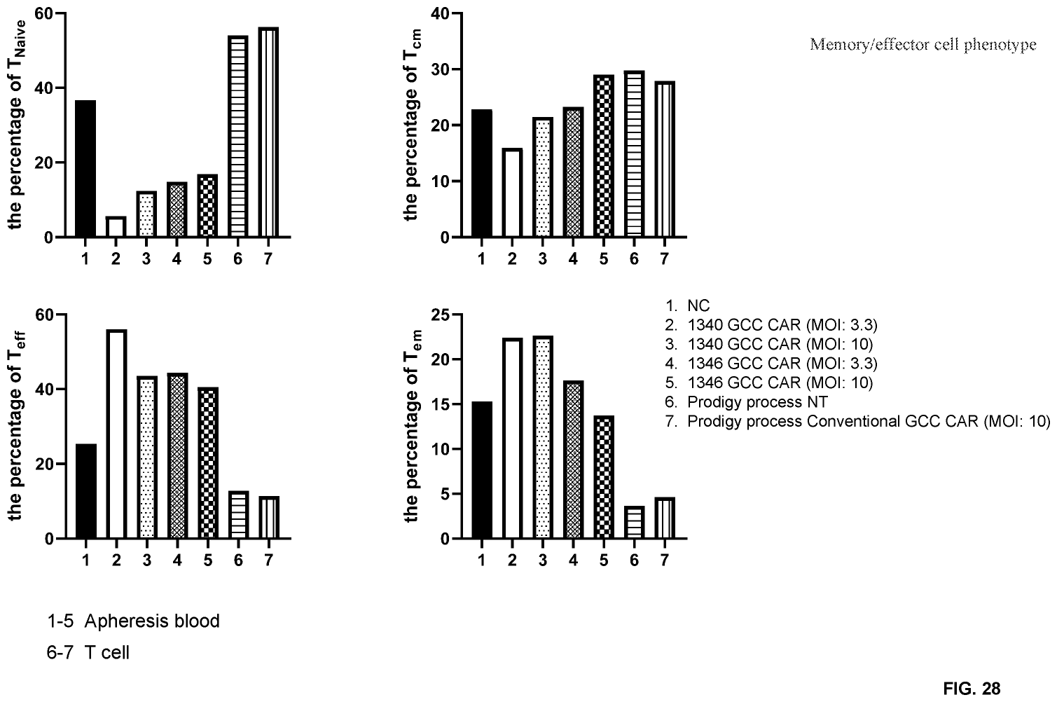

FIG. 28 shows histograms comparing the expression of different types of CAR T cells generated using various methods. (1) Prodigy's Conventional CAR T cells, produced using the Prodigy Process, are compared to T cells transduced with vectors 1346 and 1340 from a apheresis blood sample. The results show that while more Teff and Tem cells were produced by transduction with 1346 and 1340, the percent of Tn cells produced decreased. (2) The phenotype of T cells from the apheresis blood sample may have been affected by blood and other cell factors, resulting in a decrease in Tn cells and an increase in Teff and Tem cells.

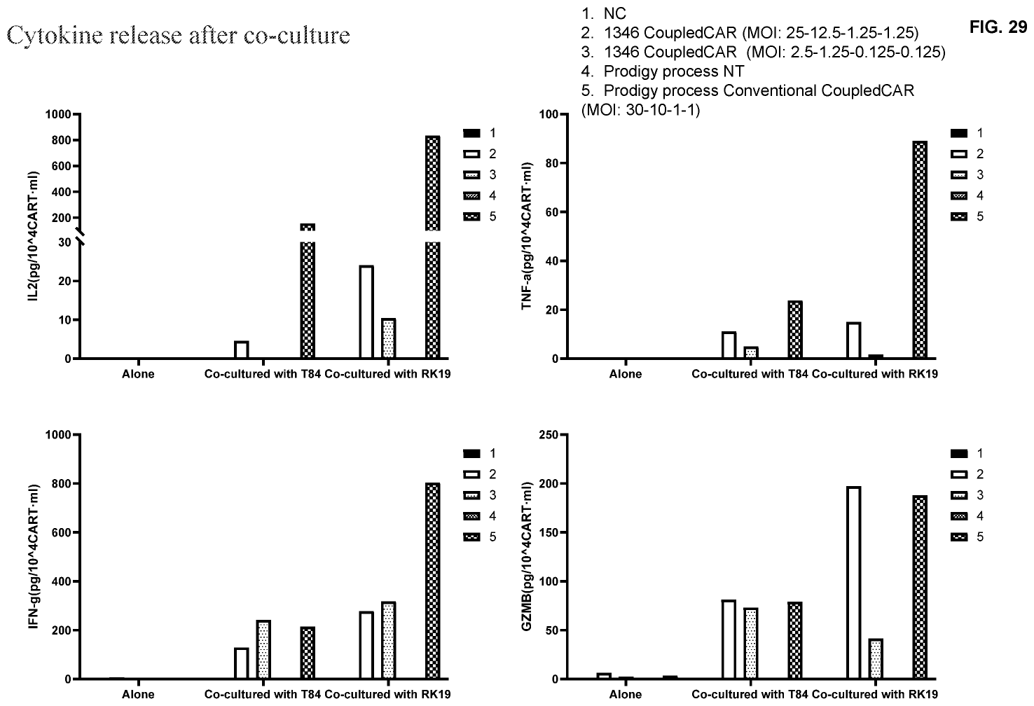

FIG. 29 shows histograms comparing cytokine release by CAR T cells generated using various methods. T cells from apheresis blood transduced with CAR vectors and packaging cell-activating element 1346 can recognize corresponding target cells, kill cells, and release factors, such as cytokines.

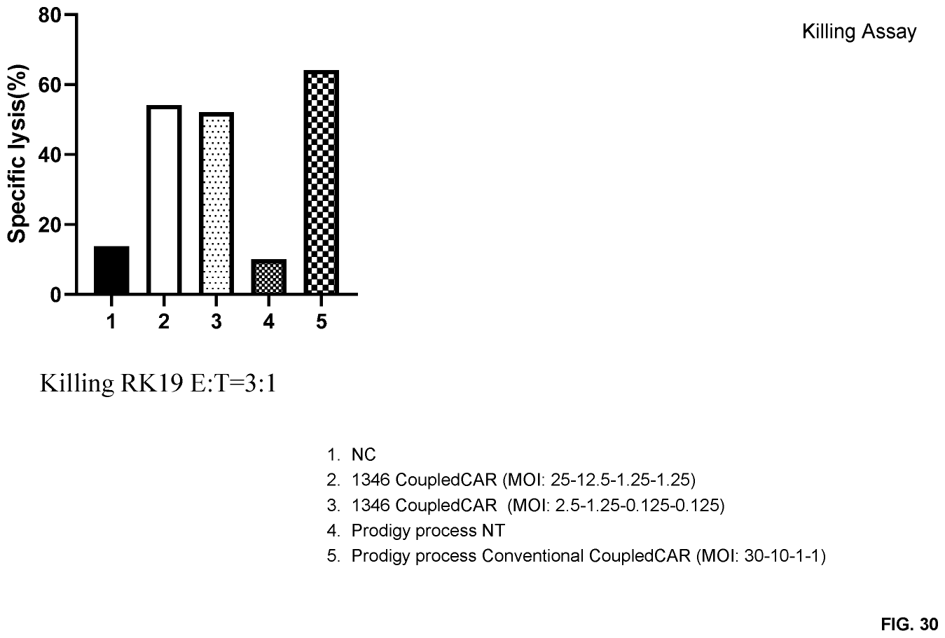

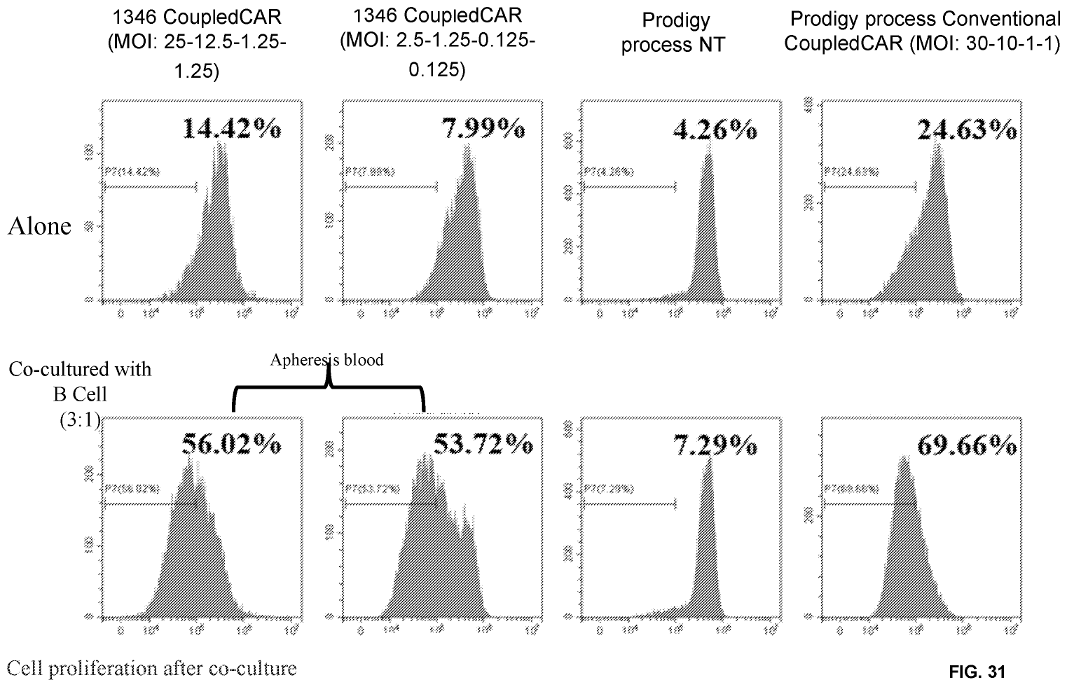

FIG. 30 shows a histogram comparing the killing ability of CAR T cells generated using various methods. T cells from s apheresis blood transduced with CAR vectors and packaging cell-activating element 1346 can recognize and kill target cells. FIG. 31 shows flow cytometry results comparing the proliferation capacity of CAR T cells generated using various methods. Carrier modified in 1346, T cells from s apheresis blood transduced with CAR vectors and packaging cell-activating element 1346 can recognize target cells and activate T cells to proliferate.

Related sequences, compositions, and methods of treating cancer are provided in this Application and Innovative Cellular Therapeutics' PCT Patent Publication NOS: WO2016138846, WO2018126369, WO2017167217, WO2019140100, WO2020146743, WO2021216731, WO2020106843, WO2020047306, and WO2022150831 and US Patent Publication NOS: US20210060069 and US20210100841, which are incorporated by reference in their entirety.

### Example 2: Novel CAR Technology and CoupledCAR® CAR T Therapy

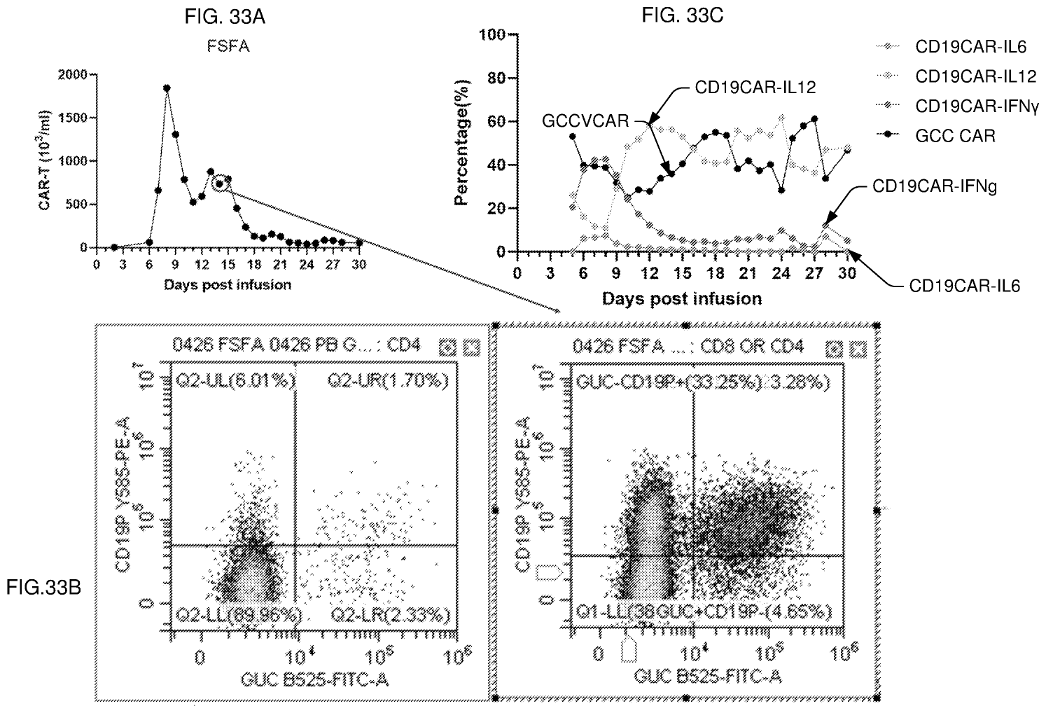

Table 4 shows the 16 different types of CAR T cells that can be generated using CoupledCAR® CAR T therapy as described herein. To further investigate the fate of these 16 types of CAR T cells in the CAR T therapy, 6 patients were selected, and their blood samples were collected for single cell sequencing analysis. Among these 6 patients, 3 of them were responders, while there other three were non-responders. For the 3 responders, their Best Overall Response (BOR) is partial remission (PR) for colorectal cancer or reduced PSA level for prostate Cancer. FIG. 32 shows a summary of CAR T therapy on Patient 01. FIGS. 33A-33C show a specific type of T cells alone was enriched after CAR T cells infusion into Patient 01.

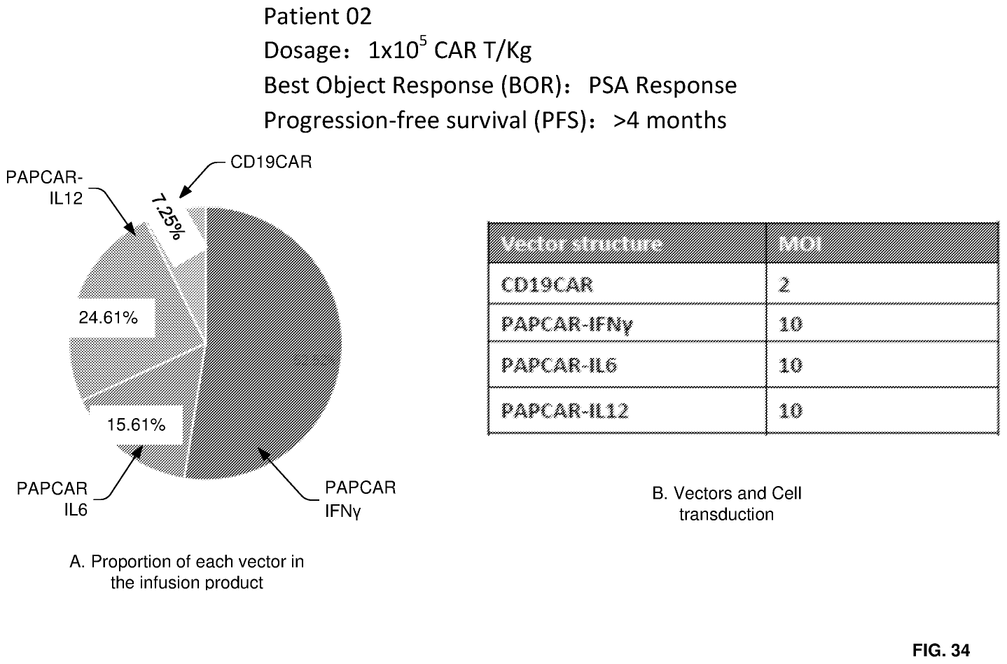

FIG. 34 shows a summary of CAR T therapy on Patient 02.

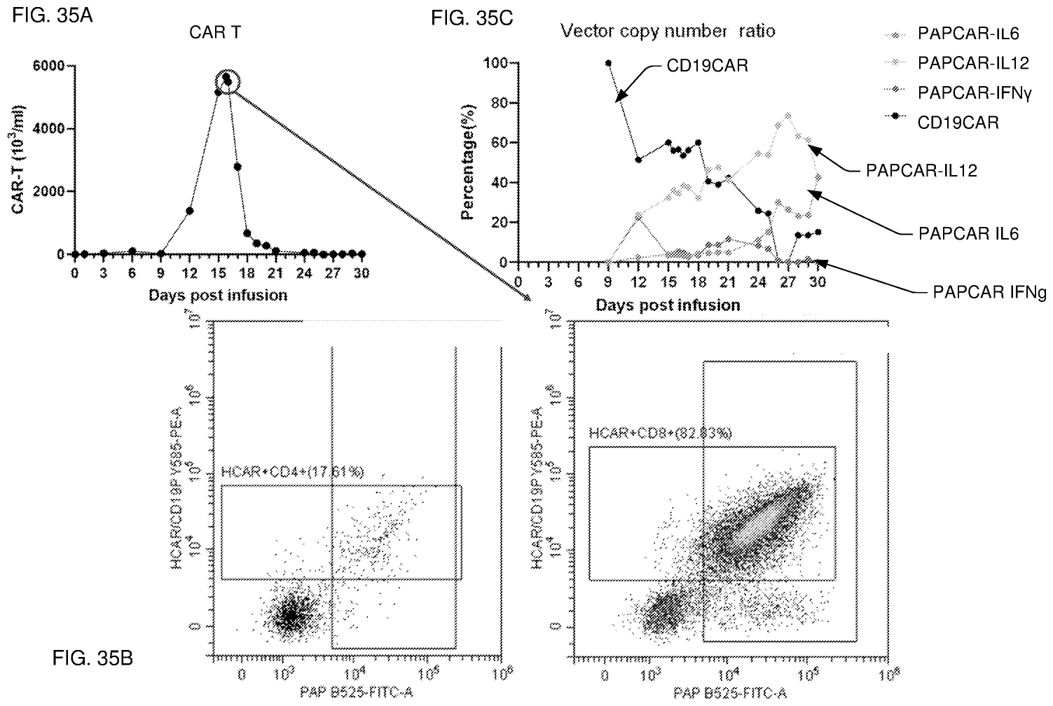

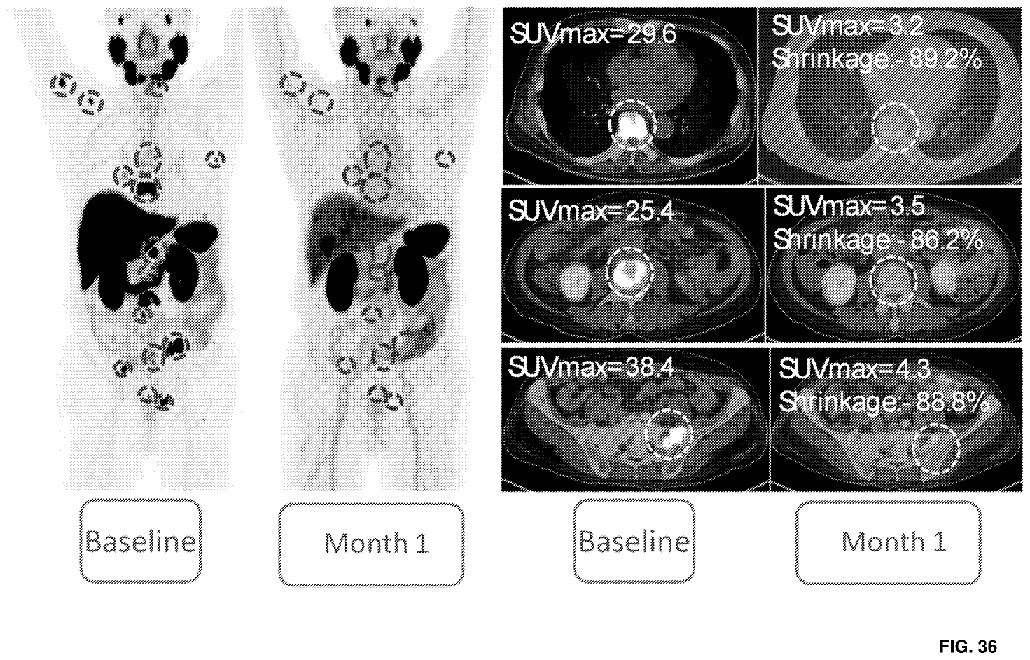

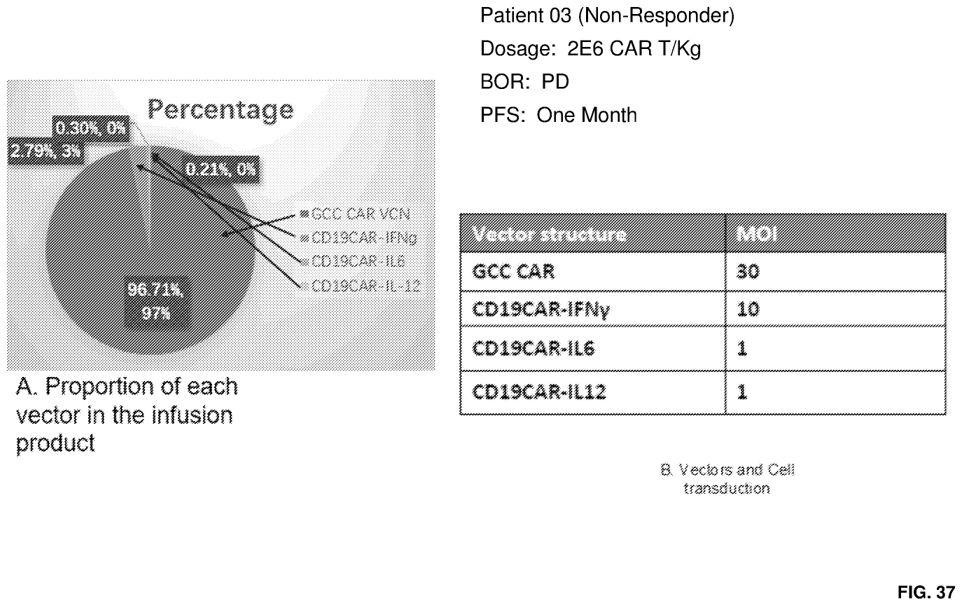

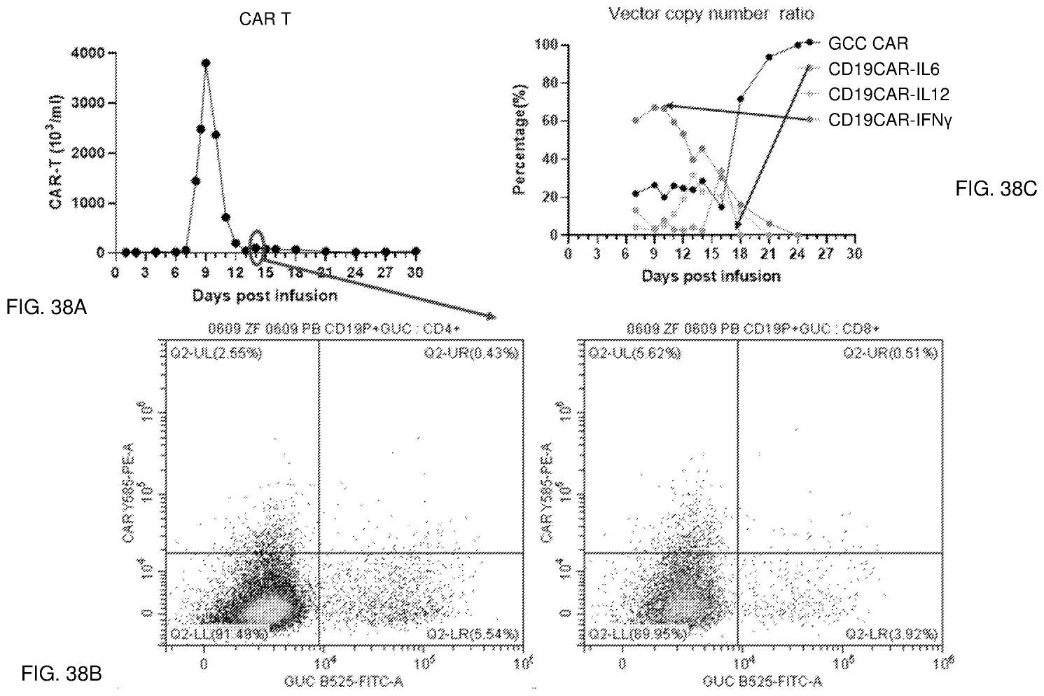

FIGS. 35A-35C shows a specific type of T cells alone was enriched after CAR T cells infusion into Patient 02. FIG. 36 shows a comparison of PET-CT scanning images of Patient 02 before and after the CAR T treatment. These results show that patients who have shown a response to the CoupledCAR® CAR T therapy, one specific type of T cells (i.e., Embodiment 5402 in FIG. 16) is significantly enriched in the patients' blood. In this case, CAR 1 is CD19 CAR, CAR 2 is PAP or GCC CAR, and cytokine is IL-12. However, this specific type of T cells was not enriched in non-responders (FIG. 39).

### Example 3: CAR on Mouse Model

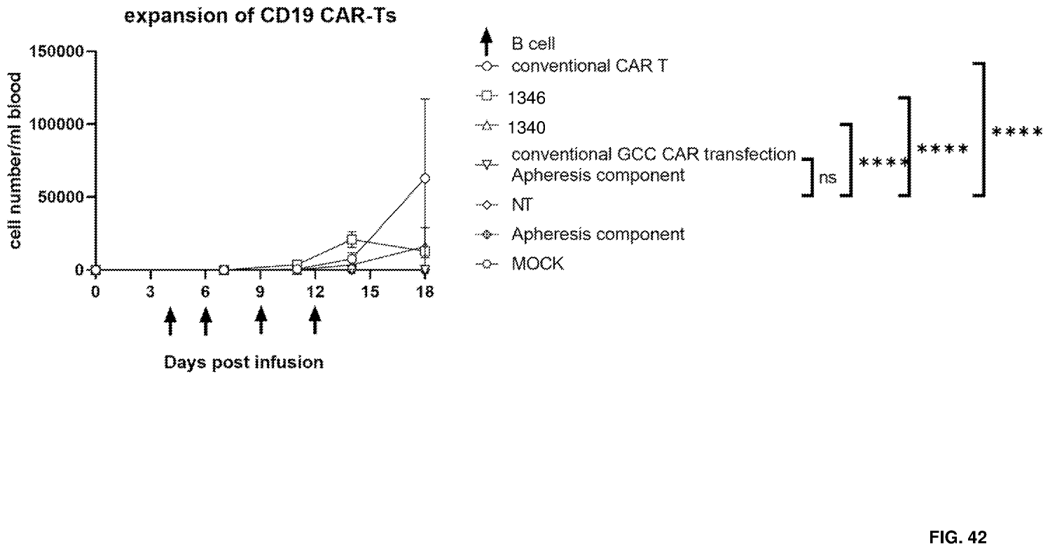

FIG. 42 shows the results of a mouse experiment, implemented using the Cellular Therapeutic Instrument 2400. The results demonstrate that both the conventional CAR T (developed through Prodigy technology) and modified transduction with packaging cell-activating element 1340 or 1346 and CAR vectors can significantly enhance the in vivo expansion of CD19 CAR T cells through B cells. Notably, packaging cell-activating element 1346 outperformed 1340.

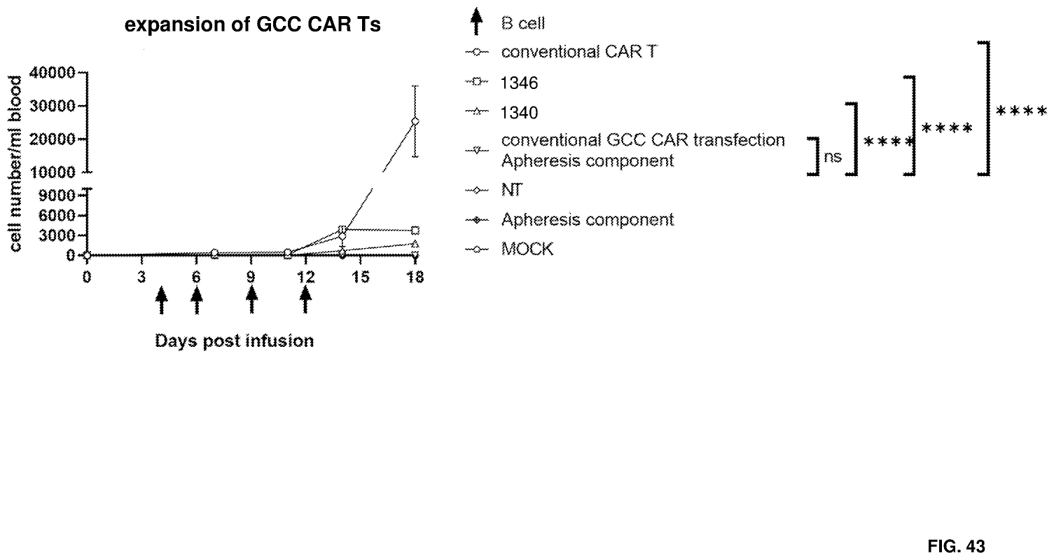

FIG. 43 shows the results of a mouse experiment, implemented using the Cellular Therapeutic Instrument 2400. The results show that both the conventional CAR T (via Prodigy technology) and modified transduction with packaging cell-activating element 1340 or 1346 and CAR vectors can significantly enhance the in vivo expansion of GCC CAR T cells through B cells. Packaging cell-activating element 1346 performed better than 1340.

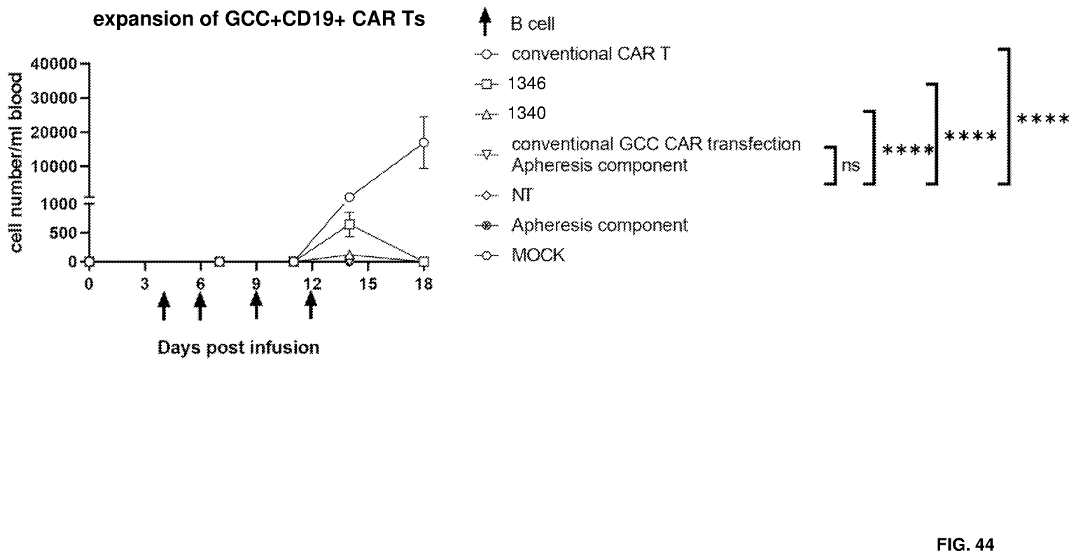

FIG. 44 shows the results of a mouse experiment, implemented by the Cellular Therapeutic Instrument 2400. The results indicate that both the conventional CAR T (Prodigy technology) and modified transduction with packaging cell-activating element 1340 or 1346 and CAR vectors can significantly amplify the dual-positive cells of GCC CAR and CD19 CAR in vivo via B cells. Once again, packaging cell-activating element 1346 exhibited superior performance over 1340.

CAR Preparation: On the day of reinfusion (DAY 0), after thawing the frozen blood obtained through apheresis with the use of Cellular Therapeutic System 2400, a combination of Conventional CAR, CAR-1340, and CAR-1346 was mixed with the thawed blood at a MOI ratio of 25-12.5-1.25-1.25. The composition was mixed for 3 minutes, washed three times, and finally packaged in doses of 1×10⁶ GCC CAR+ T cells per mouse.

Conventional CAR T Cell Preparation: T cells were obtained from frozen apheresis by CD4/8 beads selection. After stimulating with Transact for 24 hours, they were infected with 6701(GCC CAR)+6239(CD19CAR-IFNγ)+6213(CD19CAR-IL6)+6277(CD19CAR-IL12) at a MOI ratio of 30-10-1-1. The cells were then cultured until DAY 7, and the CAR expression on CAR T cells was tested via flow cytometry and stored.

On DAY 0, using Cellular Therapeutic Instrument 2400, the prepared samples of blood cells transduced with CAR were reinfused intravenously into female NOG mice (immunodeficient mouse model) aged 4-5 weeks, at a dose of 1×10⁶ GCC CAR+ T cells per mouse. Each mouse is also reinfused with 1×10⁶ B cells on DAY4, DAY 6, DAY 9, and DAY12. Blood samples were taken from the mice on DAY 7, DAY 11, DAY 14, and DAY 18 for flow cytometry to measure in vivo cell proliferation. The crucial role of the Cellular Therapeutic Instrument 2400 in this process emphasizes its importance in the implementation of this in vivo experiment.

These results shown in FIGS. 42-44 indicate that a specific type of cell (GCC CAR, CD19CAR, or GCC CAR+CD19CAR) percentage was significantly higher in the patient's blood after infusion than in the sample of blood cells being infused into the patient. This suggests that the specific type of cell, that was enriched, can play a critical role in the effectiveness of the CoupledCAR product. On the other hand, in patients who did not show any response to the infused sample of blood cells, no specific type of cell was enriched or was significantly enriched in the patients' blood. This suggests that the lack of enrichment of a specific type of cell may be associated with the lack of response to the product.

### Example 4: Modified Lentiviral Particle

On Day 0, frozen apheresis blood was thawed. Following a cell count, cells were infected with various lentiviral vectors at an MOI of 20 for 3 minutes. The sample of blood was washed three times and adjusted to a density of 1×10⁶/mL using Texmacs culture medium before being placed into a 37° C. incubator. The medium was exchanged the following day and each day afterwards until day 8. The expression of GCC CAR was tested, and a co-cultivation with target cells (T84) was performed at a ratio of 3:1 (E:T). The next day, cellular expression activation and factor release were assessed.

These lentiviral vectors were generated using the following method. Preparation of Transfer Vector Plasmids: The vector plasmids were prepared by incorporating specific genes into a DNA molecule or plasmid. For instance, in the case of 6701-1346-1485, the plasmid were constructed from the 6701 construct (encoding for GCC CAR), the 1346 construct (comprising cell-activating element gene), and a mutated variant of pLP-mVSVG (comprising mutations K47Q, R354A) encoded by the nucleotide sequence in construct 1485. For other variants such as 6701-1641-1485, 6701-1645-1485, and 6701-1646-1485, the 1346 construct was substituted with 1641, 1645, or 1646 construct, respectively. Each of these constructs comprises nucleotide sequence encoding a cell-activating element, such as an anti-CD3 scFv.

Transformation and Amplification: The prepared vector plasmids were transformed into bacterial cells, typically E. coli. These bacterial cells reproduce and replicate the plasmid DNA, resulting in an amplified quantity of the vector plasmids. The plasmid DNA is subsequently isolated and purified from the vector plasmids.

Lentivirus Packaging: The purified plasmids are co-transfected into a packaging cell line, HEK293T cells, alongside helper plasmids (pLP1 and pLP2) and packaging cell-activating element (vector). The molar ratio used for co-transfection is specified as follows: Transfer vector (6701 vector):VSVG or 1485:pLP1:pLP2:packaging cell-activating element (1346, 1641, 1645, or 1646 vector) at a molar ratio of 10:2:3:2:2. This results in the production of recombinant lentiviral particles within these cells.

Lentivirus Harvesting: After a designated period (typically 48-72 hours), the supernatant of the lentiviral particles were collected. The supernatant is then centrifuged and filtered to eliminate cellular debris.

Lentivirus Concentration and Purification: The filtered supernatant undergoes ultracentrifugation to concentrate the viral particles. The concentrated lentiviral particles are resuspended in a suitable buffer and are now ready for use.

Titering and Testing: The functional titer of the lentivirus was determined. Additionally, the vectors were tested for sterility, endotoxin levels, and performance in biological assays.

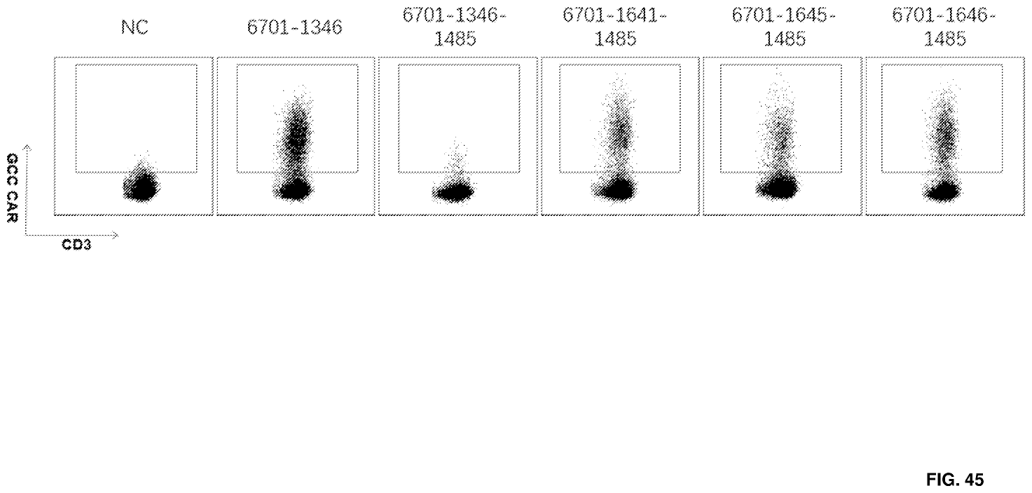

FIG. 45 illustrates CAR expression using various lentiviral particles. VSVG can bind to LDLR on the surface of T cells, and 1485 encodes a mutant variant of VSVG, which can no longer bind LDLR on the surface of T cells following the mutation. Experimental results indicate that 6701 (GCC CAR) -1346 (packaging cell-activating element)-VSVG can infect T cells in the apheresis blood. The infection rate of 6701-1346-1485 on T cells in apheresis blood significantly decreased to less than 1%. Meanwhile, replacing 1346 with 1641/1645/1646 allowed for the infection of T cells in apheresis blood.

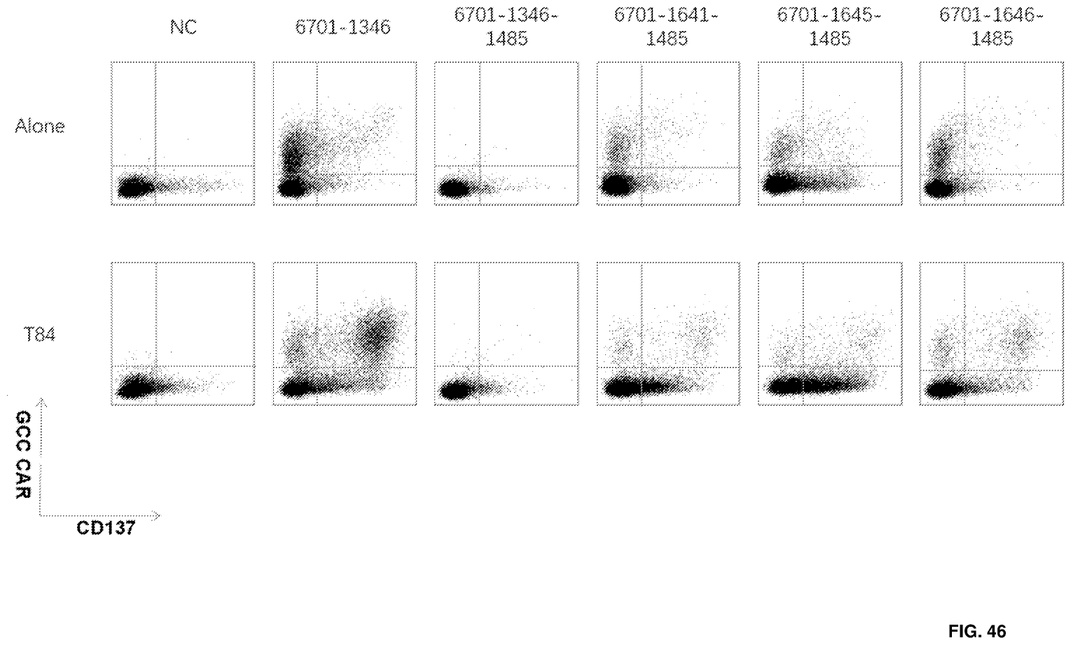

FIG. 46 shows cellular expression activation after co-cultivation. These results indicate that, compared to the control group, following co-cultivation with target cells T84, the expression of CD137 in T cells and CART cells in the apheresis blood was upregulated, indicating activation.

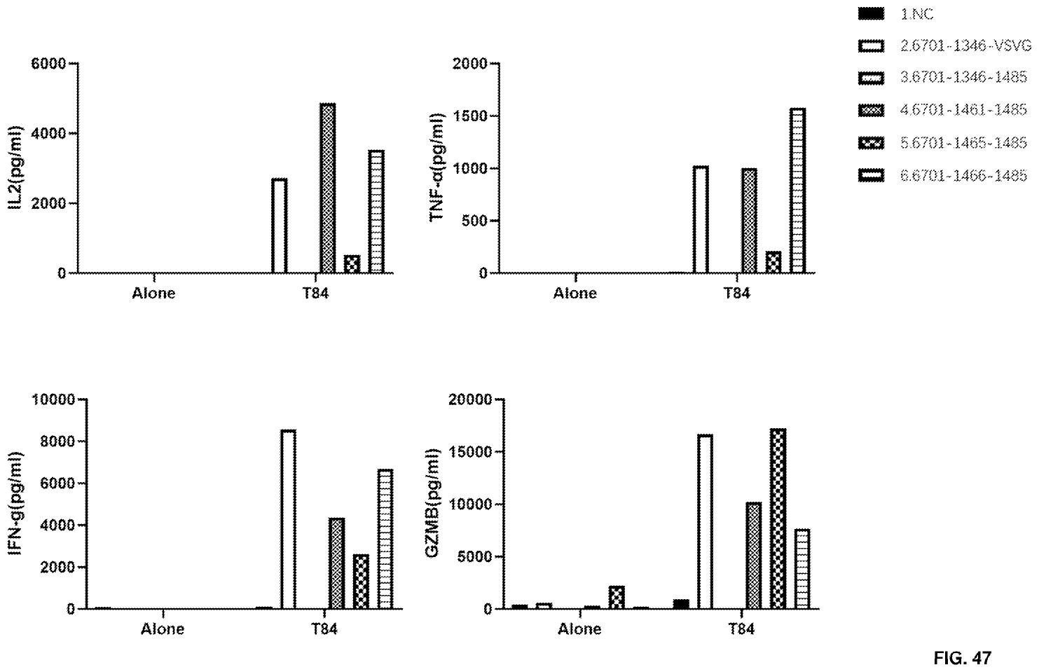

FIG. 47 shows cytokine release after co-cultivation. The results demonstrate significant cytokine release after the blood obtained from apheresis and substrate cells were co-cultivated with target cells T84, compared to the control group (without target cells).

In sum, the infection rate of 6701-1346-1485 (VSVG with LDL-R mutant) on T cells greatly declined to almost undetectable levels, suggesting that 6701-1346-VSVG is infecting through the LDL-R pathway. To ensure safety while retaining the effectiveness of the novel CAR T therapy, plasmids 1346 were replaced with 1641/1645/1646 and retained 1485-VSVG (LDL-R mutant), enabling it to infect T cells through CD3. The results confirmed that all three lentiviral vectors can infect T cells and retain normal T cell function.

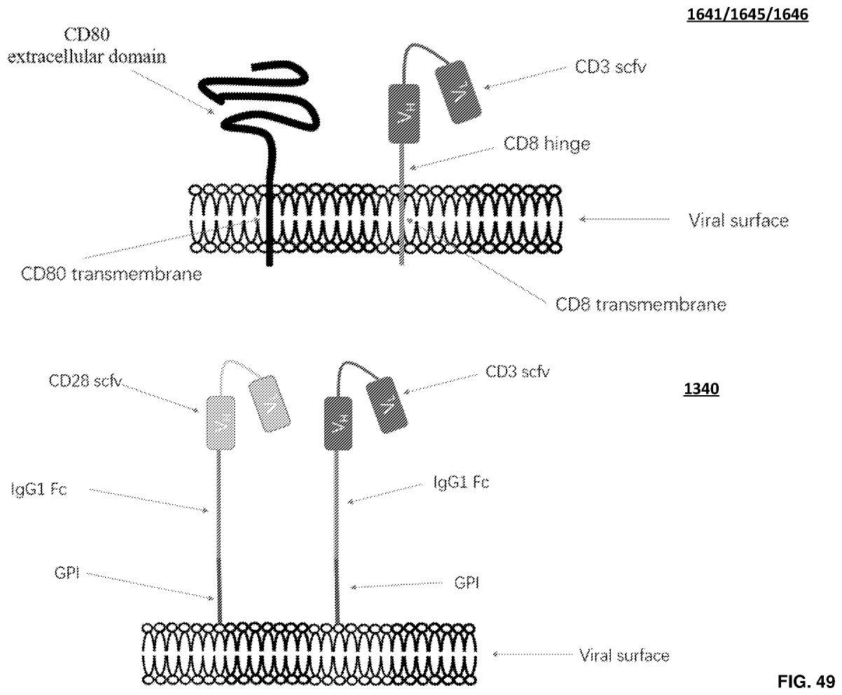

FIG. 48 shows exemplary constructs, including viral vectors. FIG. 49 shows schematic diagrams of the expression of proteins encoded by 1340 and 1641/1645/1646 on the surface of the virus.

The structures of 1641/1645/1646 include CD80 or CD28 antibodies, and CD3 antibodies, all expressed on the virus surface via a transmembrane structure. In contrast, in the 1340/1346 structure, the CD3 and CD80 or CD3 and CD28 antibodies lack a transmembrane structure. They are covalently linked to the virus surface via the last amino acid of a glycosylphosphatidylinositol (GPI) structure. This link occurs at the GPI-anchor site on the virus surface, providing a distinct mode of surface expression for these components.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

TABLE 5

| SEQ | |
|---|---|
| ID | |
| NO: | Notes |
| 1 | SP |
| 2 | Linker |
| 3 | 4-1BB |
| 4 | CD3-zeta |
| 5 | WT CD3-zeta-aa |
| 6 | scFv Humanized CD19 |
| 7 | scFv GCC |
| 8 | Anti-CD3 antibody scFv-OKT3 VH |
| 9 | Anti-CD3 antibody scFv-OKT4 VL |
| 10 | Anti-CD3 antibody scFv-F1 VH |
| 11 | Anti-CD3 antibody scFv-F1 VL |
| 12 | Anti-CD28 antibody scFv-(mAb 9.3) |
| | VH |
| 13 | Anti-CD28 antibody scFv-(mAb 9.3) |
| | VL |
| 14 | Anti-CD28 domain antibody |
| 15 | CD80 |
| 16 | CD86 |
| 17 | Anti-CD137 antibody scFv VH |
| 18 | Anti-CD137 antibody scFv VL |
| 19 | 4-1BBL |
| 20 | IL2 |
| 21 | IL7 |
| 22 | IL12 |
| 23 | IL15 |
| 24 | IL18 |
| 25 | IL21 |
| 26 | IL23 |
| 27 | CD58 |
| 28 | CD48 |
| 29 | B7-H6 (NKp30 ligand) |
| 30 | DAP12 |
| 31 | MHC class I polypeptide-related |
| | sequence A (NKG2D ligand) |
| 32 | IRES |
| 33 | P2A |
| 34 | T2A |
| 35 | E2A |
| 36 | F2A |
| 37 | CD16B GPI-anchor |
| 38 | CD14 GPI-anchor |
| 39 | CD55 GPI-anchor |
| 40 | CD59 GPI-anchor |
| 41 | CD55 signal peptide |
| 42 | IL2 signal peptide |
| 43 | TPA signal peptide |
| 44 | vesicular stomatitis virus G protein |
| | (VSV-G) |
| 45 | Moloney measles virus (Mo-MV) |
| | glycoproteins hemagglutinin (H) |
| 46 | Moloney measles virus (Mo-MV) |
| | glycoproteins fusion protein (F) |
| 47 | H mutation -HΔ18 |
| 48 | F mutation -FΔ30 |
| 49 | amphotropic murine leukemia |
| | virus (MLV-A) SU envelope |
| | glycoprotein |
| 50 | CD3antibody-MLV |
| 51 | VSVG |
| 52 | 494 |
| 53 | 1294 |
| 54 | 1297 |
| 55 | 1322 |
| 56 | 1331 |
| 57 | 1332 |
| 58 | Anti-CD3 scFv |
| 59 | Anti-CD28 scFv |
| 60 | Fc |
| 61 | CD80 ECD |
| 62 | IRES |
| 63 | IL7 |
| 64 | DAF |
| 65 | 1340AA |
| 66 | GPI attachment 1 |
| 67 | GPI attachment 2 |
| 68 | GPI attachment 3 |
| 69 | P2A |
| 70 | T2A |
| 71 | E2A |
| 72 | 1346-1 |
| 73 | 1346-2 |
| 74 | 1346-3 |
| 75 | 1347-1 |
| 76 | 1347-2 |
| 77 | 1347-3 |
| 78 | 1348-1 |
| 79 | 1348-2 |
| 80 | 1348-3 |
| 81 | 1519 |
| 82 | 1520 |
| 83 | 1521 |
| 84 | 1641 |
| 85 | 1645 |
| 86 | 1646 |
| 87 | 1485 |
| 88 | 1346 DNA: including 1346-1, |
| | 1346-2, and 1346-3 |
| 89 | 1346-4 |
| 90 | 1346-4 |
| 91 | CD8 signal peptide |
| 92 | Anti-CD3 scFv |

1. A cellular therapeutic system for treatment of a subject comprising,
a blood exchange module for collecting blood from a subject and optionally for infusing blood back into the subject;
a blood processing module for separating blood components to obtain selected blood cells from the rest of the blood; and
a cell processing module for transducing the selected blood cells with one or more viral particles comprising one or more cell-activating agents;
wherein,
the blood exchange module comprises a first outlet port with a tube connecting to the blood processing module for flowing the blood collected from the subject to the blood processing module;
the blood processing module comprises a first outlet port with a tube connecting to the cell processing module for flowing the selected blood cells to the cell processing module, and optionally, a second outlet port with a tube connecting to the blood exchange module for flowing the rest of the blood to the blood exchange module;
the cell processing module comprises an optional outlet port with a tube connecting to the blood exchange module for flowing the transduced blood cells to the blood exchange module; and
optionally, the blood exchange module comprises a second outlet port for infusing the transduced blood cells to the subject, and optionally for infusing the rest of the blood to the subject.

2. The system of claim 1, wherein the system completes the treatment of the subject in 30 minutes (mins) to 24 hrs, 30 mins to 5 hrs, 30 mins to 3 hrs, 1 hr to 24 hrs, 2 to 4 hrs, 2 to hrs, 5 to 24 hrs, 5 to 20 hrs, 5 to 16 hrs, 5 to 10 hrs, or 5 to 8 hrs.

3. The system of claim 1, wherein the blood exchange module comprises one or more kits or apparatus for collecting blood from the subject and optionally for infusing blood into the subject.

4. The system of claim 1, wherein the blood processing module comprises an apparatus for separating blood, and optionally, the apparatus is an apheresis machine.

5. The system of claim 1, wherein the cell processing module comprises an apparatus for blood transduction.

6. The system of claim 5, wherein the apparatus for blood transduction comprises one or more storage bags for storing the selected blood cells, storing the one or more viral particles for transduction, mixing the selected blood cells with the one or more viral particles, storing infusion media, and storing the transduced blood cells.

7. The system of claim 6, wherein the apparatus for blood transduction further comprises a wash chamber for washing away non-binding virus particles, and optionally wherein the apparatus for blood transduction further comprises a waste bag for collecting the non-transduced cells.

8. The system of claim 6, wherein the apparatus for blood transduction further comprises a device for culturing the transduced blood cells.

9. The system of claim 8, wherein the apparatus for blood transduction further comprises a device for exchanging the culture media of the transduced blood cells with a media for infusion.

10. The system of claim 1, wherein the system further comprises one or more mixers, fluid flow controllers, valves, luer connectors, and/or clamps.

11. The system of claim 1, wherein the one or more viral particles comprise one or more polynucleotides encoding chimeric antigen receptors (CARs), and optionally wherein the one or more CARs bind a solid tumor antigen.

12. The system of claim 1, wherein the one or more cell-activating agents comprise a signaling agent 1, a signaling agent 2, and/or a signaling agent 3, and the signaling agent 1 comprises or is a CD3 agonist, the signaling agent 2 comprises or is a CD28 agonist, 4-1 BBL, OX40L, CD86, and/or CD80, and the signaling agent 3 comprises or is one or more cytokines, optionally IL7, ILS, IL2, and/or IL12.

13. The system of claim 12, wherein the CD3 agonist comprises CD3 antibody and the CD28 agonist comprises CD28 antibody, CD86, or CD80.

14. The system of claim 1, wherein the one or more viral particles comprise SEQ ID NO: 72, SEQ ID NO: 73, SEQ ID NO: 74, SEQ ID NO: 164184, SEQ ID NO: 85, SEQ ID NO: 86, SEQ ID NO: 87, or a combination thereof.

15. The system of claim 1, wherein the one or more viral particles comprise SEQ ID NO: 88 or SEQ ID NO: 90.

16. The system of claim 1, wherein the one or more viral particles comprise VSVG, Gag, Pol, Rev, or a combination thereof.

17. The system of claim 1, wherein the selected blood cells comprise peripheral blood mononuclear cells (PBMCs), and optionally wherein the PBMCs comprise T cells.

18. The system of claim 1, wherein the system is configured to operate in a closed and automatic manner.

19. A viral particle comprising or a polynucleotide encoding SEQ ID NO: 72, SEQ ID NO: 73, SEQ ID NO: 74, SEQ ID NO: 84, SEQ ID NO: 85, SEQ ID NO: 86, SEQ ID NO: 87 or a combination thereof.

20. A viral particle comprising or a polynucleotide comprising SEQ ID NO: 88 or SEQ ID NO: 90.

21. A composition comprising the one or more viral particles of claim 19, and one or more viral particles comprising a CAR and optionally, wherein the CAR binds a solid tumor antigen.

22. A method of treating a subject having cancer comprising obtaining cells generated by the system of claim 1 and administering the generated cells to the subject.

23. A method of generating immunotherapeutic cells comprising obtaining cells generated by the system of claim 1 to generate cells and storing the cells.

24. The method of claim 22, wherein treating the subject or generating the immunotherapeutic cells is completed in 30 minutes (mins) to 24 hrs, 30 mins to 5 hrs, 30 mins to 3 hrs, 1 hr to 24 hrs, 2 to 4 hrs, 2 to hrs, 5 to 24 hrs, 5 to 20 hrs, 5 to 16 hrs, 5 to 10 hrs, or 5 to 8 hrs.

25. The method of claim 22, wherein cells are generated with a multiplicity of infection (MOI) of 1 to 10, 2 to 10, 3 to 10, 4 to 10, 5 to 10, 6 to 10, 7 to 10, 8 to 10, 9 to 10, 1 to 8, 1 to 6, 1 to 4, or 1 to 3.
